(19) United States
(12) Patent Application Publication
(10) Pub. No.: US 2009/0202528 A1
(43) Pub. Date: Aug. 13, 2009

(54) Surface-Located Streptococcus Pneumoniae Polypeptides

(76) Inventors: Thomas Kofoed, Odense (DK); Pia Nyborg Nielsen, Odense (DK); Petra Schrotz-King, Odense (DK); Jorgen Petersen, Odense (DK); Anders Boysen, Odense (DK); Tatyana A. Prokhorova, Ejby (DK); Jens Sigh, Frederiksberg (DK); Christian Morsczeck, Odense (DK)

Correspondence Address: DAVIS WRIGHT TREMAINE LLP - San Francisco, 505 MONTGOMERY STREET, SUITE 800, SAN FRANCISCO, CA, 94111, US

(21) Appl. No.: 11/884,287

(22) PCT Filed: Feb. 9, 2006
(86) PCT No.: PCT/DK06/00073

§ 371 Date: Jun. 24, 2008

Related U.S. Application Data
(60) Provisional application No. 60/653,932, filed on Feb. 18, 2005.

(30) Foreign Application Priority Data
Feb. 11, 2005 (DK) PA 2005 00207
Aug. 29, 2005 (DK) PA 2005 01194
Oct. 18, 2005 (PA) PA 2005 01463

(51) Int. Cl. A61K 39/395; C07K 16/18; C07K 14/00; C12N 15/11; C12N 15/00; C12N 7/00; C12N 1/21; A61K 38/16; A61K 39/09; A61K 35/76; A61K 31/7088; G01N 33/569; G01N 33/566; C12Q 1/18

(52) U.S. Cl. 424/133.1; 530/387.1; 530/350; 536/23.1; 435/320.1; 435/235.1; 435/252.33; 514/12; 424/190.1; 530/388.4; 530/387.3; 424/139.1; 424/93.2; 530/387.9; 424/199.1; 424/200.1; 435/7.34; 436/501; 435/32

(57) ABSTRACT

The present invention relates to cell-surface-located polypeptides of Streptococcus pneumoniae and their use in immunisation against Streptococcal infection, in diagnosis of Streptococcus and in identification of compounds with anti-Streptococcus activity. In a further aspect, the invention relates to antibodies capable of recognising cell surface-located polypeptides of Streptococcus pneumoniae and uses thereof.

All patent and non-patent references cited in this application are hereby incorporated by reference in their entirety. This patent application claims the benefit of priority from U.S. Provisional Application Ser. No. 60/653,932, filed Feb. 18, 2005, which is incorporated herein by reference in its entirety.

### FIELD OF THE INVENTION

The present invention relates to cell-surface-located polypeptides of Streptococcus pneumoniae and their use in immunisation against Streptococcal infection, in diagnosis of Streptococcus and in identification of compounds with anti-Streptococcus activity.

### BACKGROUND OF THE INVENTION

### Occurrence of Streptococcus Infections

Sternberg and Pasteur were the first to identify Streptococcus pneumoniae, initially described as the pneumococcus (Austrian R. The pneumococcus at the millennium: not down, not out. J Infect Dis 1999; 179 (Suppl 2):S338-41). Streptococcus pneumoniae is a Gram-positive encapsulated coccus. Based on differences in the composition of the polysaccharide capsule, about 90 serotypes are identified. This capsule is an essential virulence factor. The majority of pneumococcal disease in infants is associated with a small number of these serotypes, which may vary by region. Current data suggest that the 11 most common serotypes cause at least 75% of invasive disease in all regions.

Streptococcus pneumoniae is a human pathogen. The reservoir for pneumococci is presumably the nasopharynx of asymptomatic human carriers. There is no animal or insect vector. Streptococcus pneumoniae is the most common cause of bacteremia, pneumonia, meningitis and otitis media in young children. Pneumococcal disease is a very serious illness in young children. In the United States it is estimated that Streptococcus pneumoniae cause 200 deaths, 700 cases of meningitis, 17,000 cases of bacteremia, 4.9 million cases of otitis media (ear infections) annually in children under 5 years of age. In Europe and the United States, pneumococcal pneumonia is the most common community-acquired bacterial pneumonia, estimated to affect approximately 100 per 100 000 adults each year. The corresponding figures for febrile bacteremia and meningitis are 15-19 per 100,000 and 1-2 per 100,000, respectively. The risk for one or more of these manifestations is much higher in infants and elderly people.

Meningitis is the most severe type of pneumococcal disease. Of children under 5 years with pneumococcal meningitis, about 5% will die of their infection and others may have long-term problems such as hearing loss. Many children with pneumococcal pneumonia or blood stream infections will be ill enough to be hospitalized; about 1% of children with blood stream infections or pneumonia with a blood stream Infection will die of their illness. Nearly all children with ear infections recover, although children with recurrent infections can suffer hearing loss.

At serious risk are also patients taking immunosuppressive chemotherapy, those with congenital and acquired immune deficiency (including HIV infections) and those with chronic renal disease. Table. 1: The major disease indications and the number of hospitalised patients as well as case fatality rates in children and the elderly, which occur per annum in the US:

| | Pneumococcal | Pneumococcal | Pneumococcal |
|---|---|---|---|
| Disease indication | pneumonia | bacteraemia | meningitis |
| Hospitalised patients/ | 175.000 | 50.000 | 3.000-6.000 |
| annum in the US | | | |
| Case fatality rate | 5-7%/higher | 20%/60% | 30%/80% |
| children/elderly | | | |

TABLE 2
Incidence, case-fatality ratio, projected U.S. cases and deaths, and proportion nonsusceptible to
penicillin of invasive disease identified in the Active Bacterial Core surveillance (ABCs), 1998

| | Group A | Group B | Haemophilus | Neisseria | Streptococcus |
|---|---|---|---|---|---|
| | Streptococcus | Streptococcus | influenzae | meningitidis | pneumoniae |
| Aggregate incidence^{a} | 3.8 | 6.5 | 1.4 | 1.0 | 24.1 |
| Rauge by area^{a} | 2.6-4.1 | 4.8-8.5 | 1.1-2.3 | 0.6-2.0 | 20.0-28.9 |
| Case- fatality ratio | 12.2% | 9.5% | 13.9% | 13.7% | 9.3% |
| in ABCs areas | | | | | |
| Projected U.S. cases | 10,200 | 17.400 | 3.900 | 2.500 | 63,000 |
| Projected U.S. deaths | 1,300 | 1,700 | 500 | 400 | 6,100 |
| Penicillin nonsusceptibility^{b} | 0 | 0 | — | 1.1% | 25.0% |

^{a}Incidence = cases per 100,000.
^{b}Nonsusceptible includes isolates classified as either intermediate or resistant to penicillin. Results reflect testing of group A streptococcal isolates from 1997 (n = 183) and group B streptococcal isolates from 1997 and 1998 combined (n = 188).

Schuchat, A et al. “Active Bacterial Core Surveillance of the Emerging Infections. Program Network”, Emerging Infectious Diseases, Vol. 7, No. 1, January-February 2001.

Symptoms of Streptococcus pneumoniae Infections

Pneumococcal pneumonia is the most common clinical presentation of pneumococcal disease among adults. The incubation period of pneumococcal pneumonia is short, about 1 to 3 days. Symptoms generally include an abrupt onset of fever and chills or rigors. Typically there is a single rigor, and repeated shaking chills are uncommon. Other common symptoms include pleuritic chest pain, cough productive of mucopurulent, rusty sputum, dyspnea (shortness of breath), tachypnea (rapid breathing), hypoxia (poor oxygenation), tachycardia (rapid heart rate), malaise, and weakness.

Treatment and Prevention of Streptococcus pneumoniae Infections

The emerging resistance to penicillin and other commonly used antibiotics underscores the importance of the development of novel strategies to combat pneumococcal disease. In some areas of the U.S. up to 40% of invasive pneumococcal isolates are resistant to penicillin. Treatment will usually include a broad spectrum cephalosporin, and often vancomycin, until results of antibiotic sensitivity testing are available.

There are two vaccines against Streptococcus pneumoniae available on the market:
1. Prevnar® (Wyeth), a 7-valent pneumococcal conjugate vaccine, containing polysaccharides of serotype 4, 6B, 9V, 14, 18C, 19F and 23F.
2. Pneumovax® (Merck Research Laboratories), a 23-valent polysaccharide vaccine containing 23 purified capsular polysaccharide antigens (serotypes 1, 2, 3, 4, 5, 6B, 7F, 8, 9N, 10A, 11A, 12F, 14, 15B, 17F, 18C, 19A, 19F, 20, 22F, 23F, and 33F).

However, there is still a large medical need for development of improved Streptococcus vaccines, because:
These vaccines only cover certain serotypes, e.g. Prevnar® has a potential coverage of over 85% of the pneumococcal isolates for the USA, 60-70% for Europe and around 55% for Asia.
Children under 2 years of age, who suffer the highest rates of pneumococcal carriage and disease, and immunocompromised patients show a severely impaired antibody response upon this vaccination.
The polysaccharide vaccines are not effective against acute otitis media caused by Streptococcus pneumoniae.
The polysaccharide vaccines do not induce a T-cell-dependent immune response. This implicates the absence of memory B cells and limits the period of protection.
Several of the capsule polysaccharides are poorly immunogenic. These include several serotypes associated with penicillin resistance.

Currently, several pneumococcal surface proteins are considered as alternative vaccine candidates because of their serotype-independence. However, so far, none of the proteins are considered to elicit species-wide pneumococcal protection. This can be explained by the occurrence of allelic variation within most individual proteins. Antibodies raised against a single protein may not recognize allelic variants. Efficacy against pneumonia is an important factor in deciding on the use of new vaccines in developing countries.

In addition to better ways of treatment and prevention, there is a need for novel rapid and reliable methods for diagnosis of Streptococcus pneumoniae infections. The above objectives can be accomplished through the identification and use of suitable Streptococcus pneumoniae polypeptides that can function as targets, i.e. targets for the immune system and/or for antibodies, targets for cytotoxic inhibitors, or targets for indicator moieties in diagnosis.

### SUMMARY OF THE INVENTION

The present application relates to surface-located polypeptides of Streptococcus pneumoniae. In the context of this application, a ‘surface-located’ polypeptide is defined as a polypeptide which is at least partially (i.e. part of the polypeptide chain and/or part of the population of polypeptide molecules) localised outside the membrane of a Streptococcus pneumoniae cell. Thus, a surface-located polypeptide is a polypeptide which is fully or partially exposed to the space outside the membrane. Surface-located polypeptides furthermore include all polypeptides or polypeptide fragments that can be identified in fractions obtained by high-pH surface-protein extraction or mutanolysin digestion as described herein.

Surface-located polypeptides are attractive targets for antibacterial therapy and/or diagnosis of bacterial infection, since the exposure of such polypeptides to the extracellular space means that compounds that interact with these polypeptides (e.g. compounds used to prevent, treat or diagnose bacterial infections) often do not need to enter or pass the membrane to be effective.

The determination of cell-surface localisation of a Streptococcus pneumoniae polypeptide can at present only be done experimentally and not by bioinformatics, as no common sorting signals or motifs are known for this localisation. It is possible to predict with some degree of certainty whether or not polypeptides enter the periplasm, but no general motif has been identified for surface-localisation of polypeptide. Prior art strategies for the identification of candidates for protein vaccination against Streptococcus pneumoniae have mainly been based on genome sequencing and in silico analysis (WO 02/077021; Wizemann et al. (2001) Infect. Immun. 69:1593-1598). These strategies have not been very successful, as only a small subpopulation of the candidates identified and tested conferred protection in a mouse model (Wizemann et al. (2001) Infect. Immun. 69:1593-1598).

The inventors have identified 282 different polypeptides in cell-surface fractions of Streptococcus pneumoniae. The method that was employed identifies polypeptides that are expressed at a relatively high level. The combination of being surface-exposed and being present in relatively high amounts makes these polypeptides highly suitable as targets for antibodies and thus for use in passive or active immunisation/vaccination.

Accordingly, in a first aspect, the invention relates to a composition comprising
a polypeptide which comprises a sequence selected from the group consisting of surface-located Streptococcus polypeptides of SEQ ID NO:1-282, or comprises an antigenic fragment or variant of said sequence,
or
a polynucleotide comprising a sequence encoding said polypeptide,
or
an expression vector comprising a sequence encoding said polypeptide,
or
a recombinant virus or recombinant cell comprising said polynucleotide or said expression vector,
or
an antibody capable of binding said polypeptide,
   for use as a medicament.

In a preferred embodiment, said composition comprises
a polypeptide which comprises a sequence selected from the group consisting of SEQ ID NO:16, SEQ ID NO:10, SEQ ID NO:13, and SEQ ID NO:28, or comprises an antigenic fragment or variant of said sequence,
or
a polynucleotide comprising a sequence encoding said polypeptide,
or
an expression vector comprising a sequence encoding said polypeptide,
or
a recombinant virus or recombinant cell comprising said polynucleotide or said expression vector.

In an even more preferred embodiment, said composition comprises
a polypeptide which comprises SEQ ID NO:16, or comprises an antigenic fragment or variant of SEQ ID NO:16,
or
a polynucleotide comprising a sequence encoding said polypeptide,
or
an expression vector comprising a sequence encoding said polypeptide,
or
a recombinant virus or recombinant cell comprising said polynucleotide or said expression vector. SEQ ID NO:16 represents a homolog of lipoate-protein ligase A, an enzyme which has previously been identified and characterised in E. coli and L. monocytogenes (Morris et al. (1994) J. Biol. Chem. 269:16091; O'Riordan et al. (2003) Science 302:462). Proteins of this family have not previously been identified on the cell surface or found to be vaccine candidates or suitable targets for antibody therapy.

In another preferred embodiment, said composition comprises an antibody capable of binding a polypeptide selected from the group consisting of SEQ ID NO:16, SEQ ID NO:10, SEQ ID NO:13, SEQ ID NO:20, SEQ ID NO:26, SEQ ID NO:28 and SEQ ID NO:33. In an even more preferred embodiment, said polypeptide is SEQ ID NO:16.

In a further main aspect, the invention relates to the use of a composition comprising
a polypeptide which comprises a sequence selected from the group consisting of SEQ ID NO:1-282, or comprises an antigenic fragment or variant of said sequence,
a polynucleotide comprising a sequence encoding said polypeptide,
an expression vector comprising a sequence encoding said polypeptide, or
a recombinant virus or recombinant cell comprising said polynucleotide or said expression vector,
   for the preparation of a medicament for the immunisation of an animal or human being against bacteria, preferably Streptococcus, more preferably Streptococcus pneumoniae, infections. Preferred sequences are SEQ ID NO:16, SEQ ID NO:10, SEQ ID NO:13, and SEQ ID NO:28. Most preferred SEQ ID NO:16.

In a further main aspect, the invention relates to an antibody capable of binding a polypeptide selected from the group consisting of SEQ ID NO:1-282.

Furthermore, the invention relates, in a main aspect, to the use of an antibody capable of binding a polypeptide selected from the group consisting of SEQ ID NO:1-282 for the manufacture of a medicament for the treatment or prevention of Streptococcus, preferably Streptococcus pneumoniae, infections in an animal or human being. The use of antibodies capable of binding a polypeptide selected from the group consisting of SEQ ID NO:16, SEQ ID NO:10, SEQ ID NO:13, SEQ ID NO:20, SEQ ID NO:26, SEQ ID NO:28 or SEQ ID NO:33 is preferred.

Most preferred is the use of an antibody capable of binding the polypeptide of SEQ ID NO:16 for the manufacture of a medicament for the treatment or prevention of Streptococcus, preferably Streptococcus pneumoniae, infections in an animal or human being.

The combination of being surface-exposed and being present in relatively high amounts also makes the polypeptides identified by the inventors highly suitable as targets for diagnosis of Streptococcus pneumoniae infection, allowing detection of intact cells with high sensitivity. Thus, in a further main aspect, the invention relates to methods for detecting Streptococcus pneumoniae or parts thereof, using indicator moieties capable of recognising the cell-surface located polypeptides described herein.

In addition, the surface-localisation of the polypeptides makes them suitable as targets for inhibitors. Such inhibitors may be bactericidal or bacteriostatic or prevent interaction of Streptococcus pneumoniae with the host organism (virulence). Thus, in a further main aspect, the invention relates to methods for identifying inhibitors of the cell-surface located polypeptides described herein.

### DEFINITIONS

Vaccine—is used to indicate a composition capable of inducing a protective immune response against a microorganism in a human being or animal.
Protective immune response—is used to indicate an immune response (humoral/antibody and/or cellular) inducing memory in an organism, resulting in the infectious agent, herein Streptococcus pneumoniae, being met by a secondary rather than a primary response, thus reducing its impact on the host organism.
Polypeptide—unless specified otherwise, the term ‘polypeptide’ when used herein can also refer to a variant or fragment of a polypeptide. Preferred polypeptides are antigenic polypeptides.
Fragment—is used to indicate a non-full length part of a polypeptide. Thus, a fragment is itself also a polypeptide.
Variant—a ‘variant’ of a given reference polypeptide refers to a polypeptide that displays a certain degree of sequence identity to said reference polypeptide, but is not identical to said reference polypeptide.
Antigen/antigenic/antigenicity/immunogen/immunogenic/immunogenicity all refer to the capability of inducing an immune response.
Immunogenic carrier—refers to a compound which directly or indirectly assists or strengthens an immune response.
Expression vector—refers to a, preferably recombinant, plasmid or phage or virus to be used in production of a polypeptide from a polynucleotide sequence. An expression vector comprises an expression construct, comprising an assembly of (1) a genetic element or elements having a regulatory role in gene expression, for example, promoters or enhancers, (2) a structural or coding sequence which is transcribed into mRNA and translated into protein, and which is operably linked to the elements of (1); and (3) appropriate transcription initiation and termination sequences.
Binding partner—of a polypeptide refers to a molecule that can bind to said polypeptide. Such binding can be indirect, through another molecule, but is preferably direct. A binding partner can be any type of molecule, such as e.g. small hydrophobic molecules or e.g. a cellular or extracellular macromolecule, such as a protein, a carbohydrate or a nucleic acid. Preferred types of binding partners include antibodies, ligands or inhibitors.
Plurality—the term ‘plurality’ indicates more than one, preferably more than 10.
Indicator moiety—the term ‘indicator moiety’ covers a molecule or a complex of molecules that is capable of specifically binding a given polypeptide and/or cell, and is capable of generating a detectable signal. Preferably, the indicator moiety is an antibody or comprises an antibody molecule. Thus, a preferred indicator moiety is an antibody coupled to or in complex with a detectable substance.
Host-derived molecule or host molecule—refers to a molecule which is normally found in a host organism that can be infected with Streptococcus pneumoniae. A host-derived molecule is preferably a host, polypeptide, preferably a human polypeptide.
Antibody—the term ‘antibodies’ when used herein is intended to cover antibodies as well as functional equivalents thereof. Thus, this includes polyclonal antibodies, monoclonal antibodies (mAbs), humanised, human or chimeric antibodies, single-chain antibodies, and also binding fragments of antibodies, such as, but not limited to, Fab fragments, F(ab′)₂ fragments, fragments produced by a Fab expression library, anti-idiotypic antibodies, hybrids comprising antibody fragments, and epitope-binding fragments of any of the these. The term also includes multivalent, multispecific, such as bispecific antibodies and mixtures of monoclonal antibodies.
Dissociation constant, Kd, is a measure to describe the strength of binding (or affinity or avidity) between macromolecules, for example an antibody and its antigen. The smaller Kd the stronger binding.

Isolated—used in connection with polypeptides, polynucleotides and antibodies disclosed herein ‘isolated’ refers to these having been identified and separated and/or recovered from a component of their natural, typically cellular, environment. Contaminant components of the natural environment are materials that would typically interfere with diagnostic or therapeutic uses for the polypeptide, and may include enzymes, hormones, and other proteinaceous or non-proteinaceous solutes. Polypeptides, polynucleotides and antibodies of the invention are preferably isolated, and vaccines and other compositions of the invention preferably comprise isolated polypeptides or isolated polynucleotides or isolated antibodies.

### DETAILED DESCRIPTION

### Figures

FIG. 1: A table of preferred compositions of the invention. The numbers in the columns and rows indicate SEQ ID NOs. Each cross refers to a composition comprising the polypeptide (or antigenic fragment or variant thereof) of the column to which the cross belongs as well as the polypeptide (or antigenic fragment or variant thereof) of the row to which the cross belongs.

FIG. 2: List of amino acid sequences of surface-located Streptococcus pneumoniae polypeptides.

FIG. 3: RT-PCR with cDNA derived from a spleen from a mouse infected with S. pneumoniae D39 at 1 day of infection. Primers were used specific for transcripts for antigens 029 (SEQ ID NO:16), 060 (SEQ ID NO:26), 607 (SEQ ID NO:20) and 653 (SEQ ID NO:33). Moreover, primers were used specific for transcript of the Sigma 70 subunit of the pneumococcal RNA-Polymerase (house keeping gene). −RT: control without reverse transcriptase; +RT: RT—PCR; N: non-template control.

FIG. 4: Immunoblot with patient serum (single patient) for detection of rec. vac. (antigens) 029, 060, 144, 487, 607, 646 and 653.

FIG. 5: Immunogenicity of antigens (ags) 029, 060, 607, 653 and controls with untreated animals (non immunized), Alum adjuvants alone, and an unrelated antigen at days 0, 21 and 35 of vaccination.

FIG. 6: CFU 6 h after challenge with S. pneumoniae D39 in blood of mice vaccinated with antigens (ags) 029, 060, 607, 653 and controls with untreated animals (non immunized), Alum adjuvants alone, and unrelated antigen.

FIG. 7: Survivors after challenge with S. pneumoniae D39 of mice vaccinated with antigens (ags) 029 and 607 compared with control groups with untreated animals (non immunized), Alum adjuvants alone and an unrelated antigen (sigma)

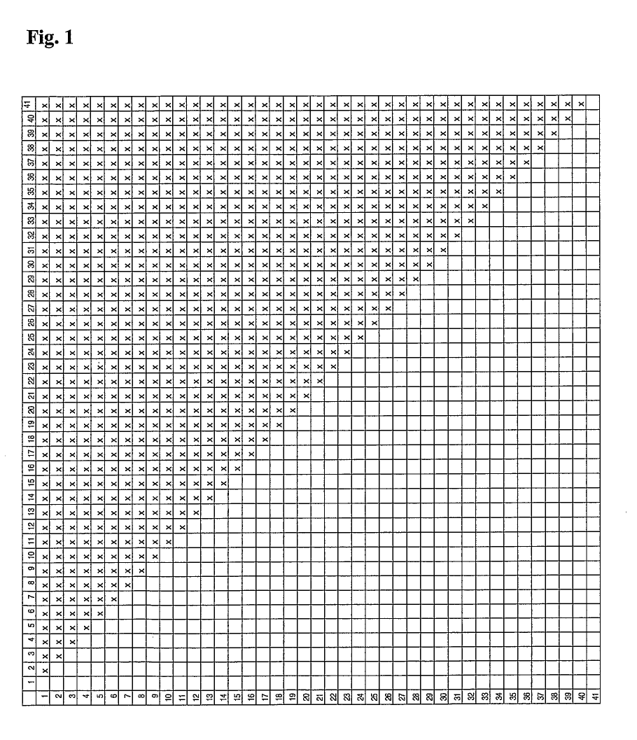

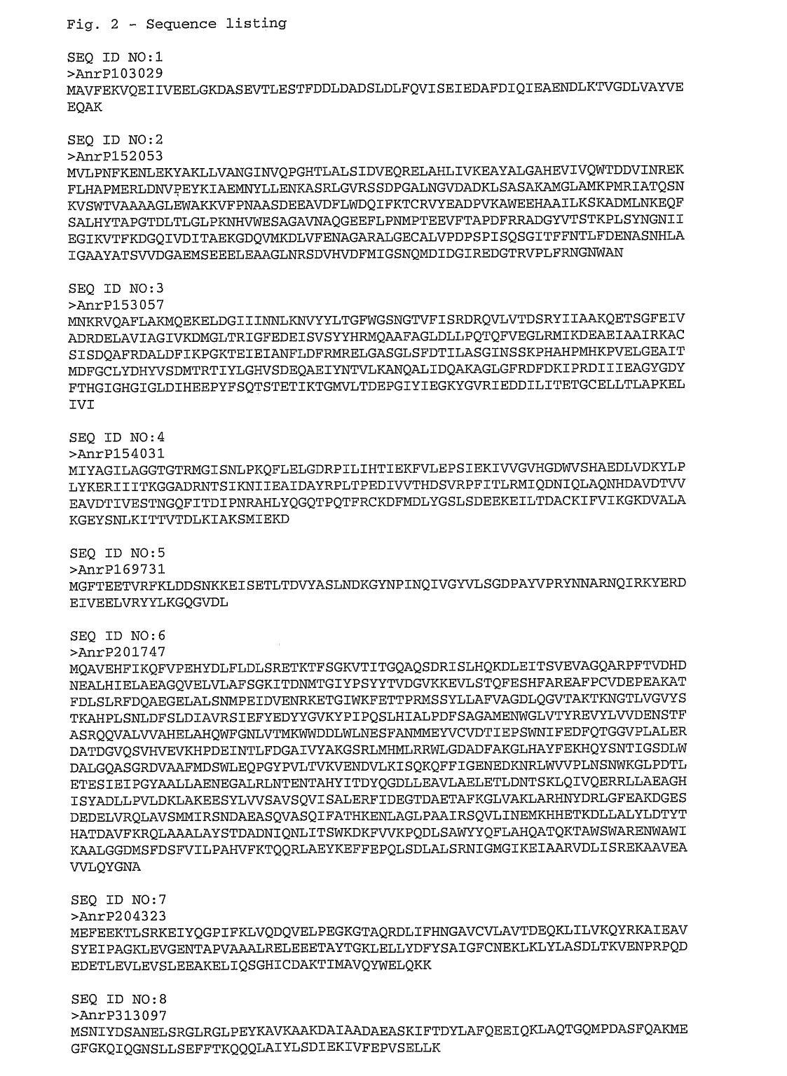

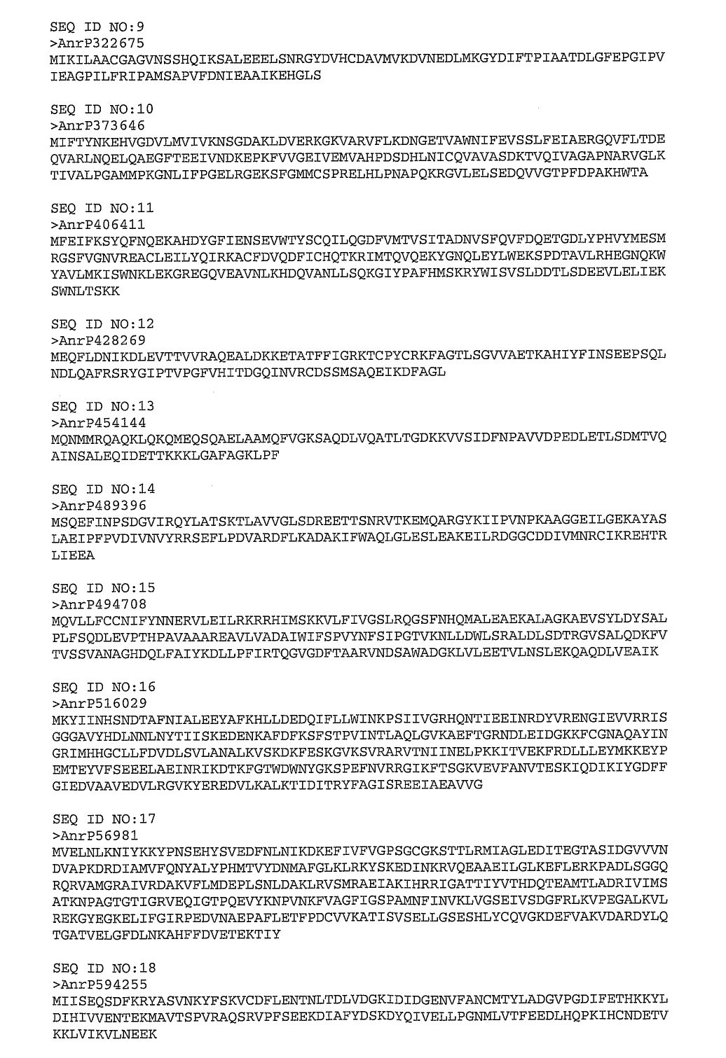

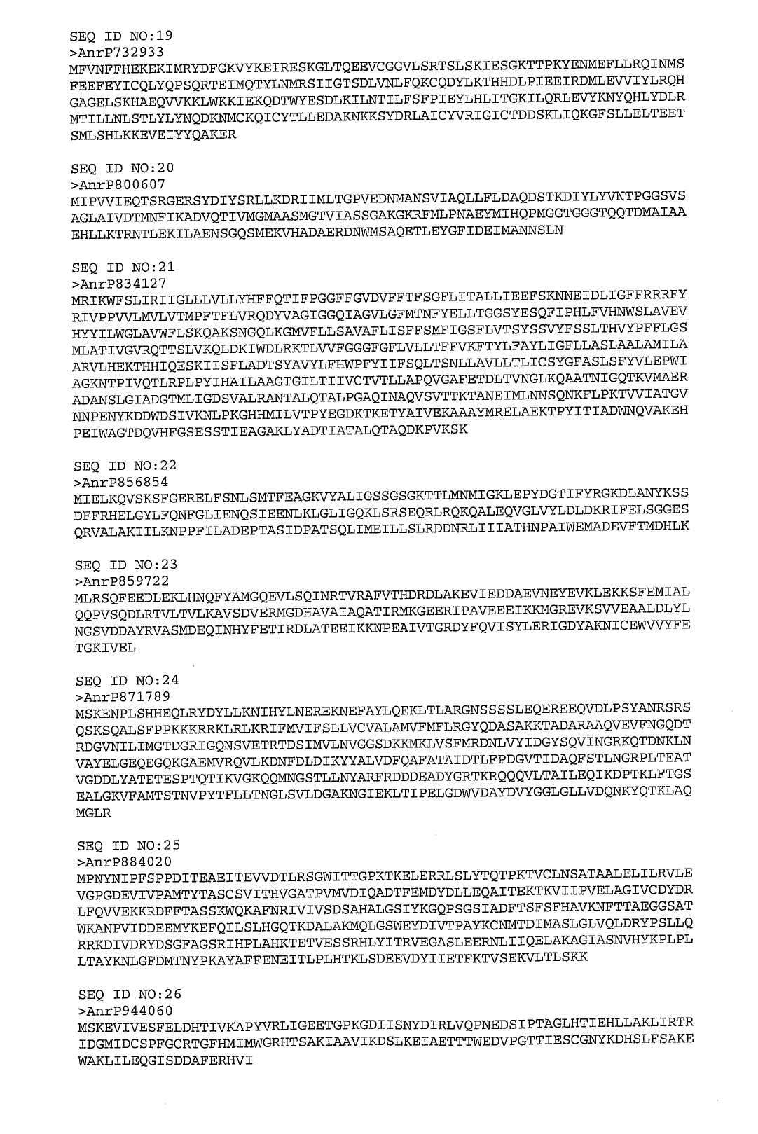

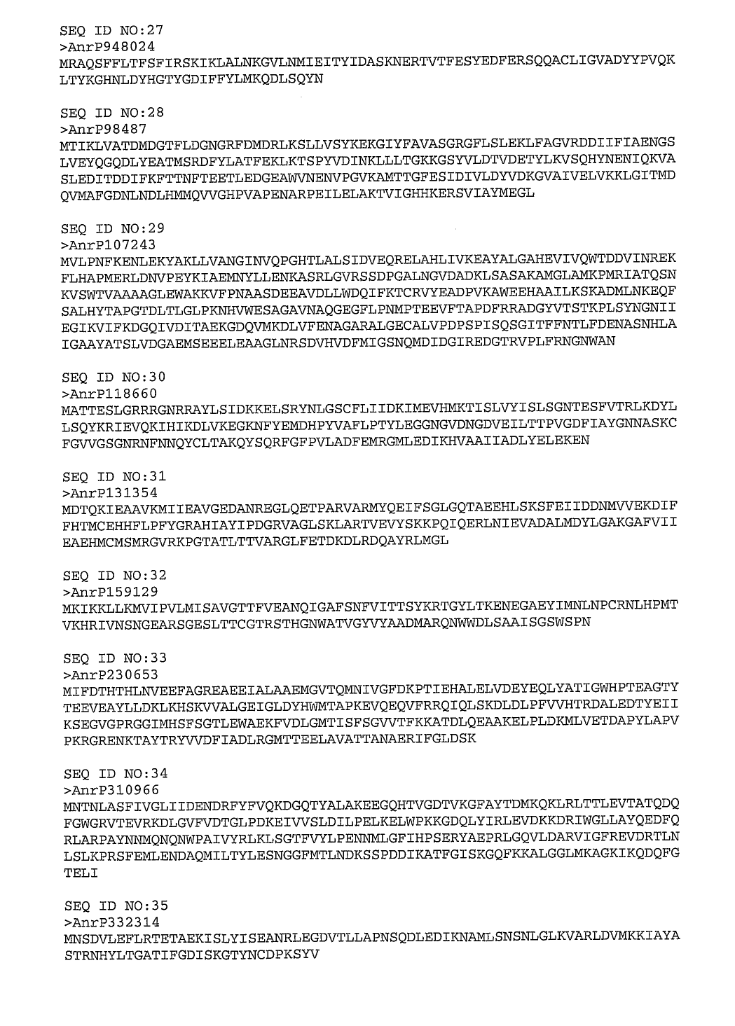

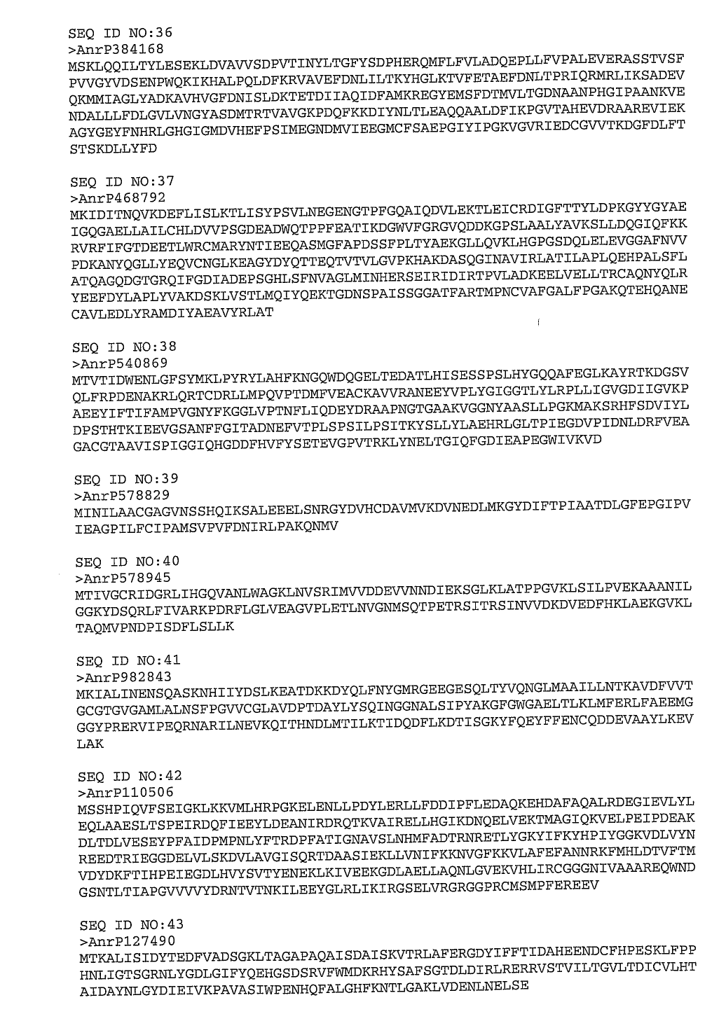

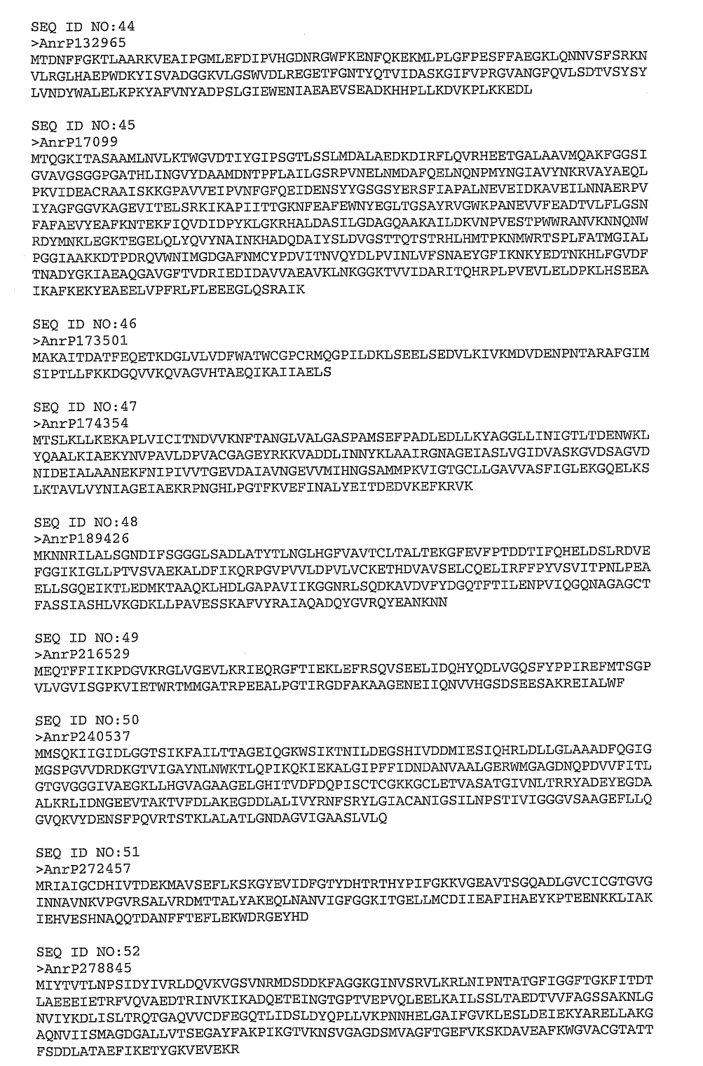

### COMPOSITIONS FOR USE AS A MEDICAMENT

In a first main aspect, the invention relates to a composition comprising
a polypeptide which comprises a sequence selected from the group consisting of surface-located Streptococcus pneumoniae polypeptides of SEQ ID NO:1-282, or comprises an antigenic fragment or variant of said sequence,
a polynucleotide comprising a sequence encoding said polypeptide,
an expression vector comprising a sequence encoding said polypeptide,
a recombinant virus or recombinant cell comprising said polynucleotide or said expression vector, or
an antibody capable of binding said polypeptide,
   for use as a medicament.

In an important embodiment, the composition comprises
a polypeptide which comprises a sequence selected from the group consisting of SEQ ID NO:1-282, or comprises an antigenic fragment or variant of said sequence,
a polynucleotide comprising a sequence encoding said polypeptide,
an expression vector comprising a sequence encoding said polypeptide, or
a recombinant virus or recombinant cell comprising said polynucleotide or said expression vector.

Said composition can be used as a vaccine for active immunisation of an individual in need thereof. This is described in the section ‘vaccine compositions and methods of vaccination of the invention’.

In one preferred embodiment, the composition comprises a polypeptide which comprises a sequence selected from the group consisting of SEQ ID NO:1-282 or comprises antigenic fragment or variant of said sequence.

In another important embodiment, the composition comprises an antibody capable of binding a polypeptide selected from the group consisting of surface-located Streptococcus pneumoniae polypeptides of SEQ ID NO:1-282. Said composition can e.g. be used in passive immunisation of an individual in need thereof. This is described in the section ‘antibodies and methods for raising antibodies of the invention’.

### Vaccine Compositions and Methods of Vaccination of the Invention

The goal of vaccination or active immunisation is to provide protective immunity by inducing a memory response to an infectious microorganism using an antigenic or immunogenic composition. Thus, a vaccine is a composition capable of inducing a protective immune response against a microorganism in a human being or animal. Such an immune response can be a cellular response and/or a humoral response, e.g. a specific T cell response or an antibody response.

Accordingly, in an important embodiment, the composition is a vaccine composition. I.e. the invention relates to the use of a composition comprising
a polypeptide which comprises a sequence selected from the group consisting of surface-located Streptococcus pneumoniae polypeptides of SEQ ID NO:1-282, or comprises an antigenic fragment or variant of said sequence,
a polynucleotide comprising a sequence encoding said polypeptide,
an expression vector comprising a sequence encoding said polypeptide, or
a recombinant virus or recombinant cell comprising said polynucleotide or said expression vector,
   as a vaccine.

The variant herein preferably has at least 95% sequence identity, such as at least 96%, e.g. at least 97%, such as at least 98%, e.g. at least 99% sequence identity to said sequence.

In one preferred embodiment of the above composition, the polypeptide comprises SEQ ID NO:1, or an antigenic fragment or variant thereof.

In another preferred embodiment of the above composition, the polypeptide comprises SEQ ID NO:2, or an antigenic fragment or variant thereof.

In another preferred embodiment of the above composition, the polypeptide comprises SEQ ID NO:3, or an antigenic fragment or variant thereof.

In another preferred embodiment of the above composition, the polypeptide comprises SEQ ID NO:4, or an antigenic fragment or variant thereof.

In another preferred embodiment of the above composition, the polypeptide comprises SEQ ID NO:5, or an antigenic fragment or variant thereof.

In another preferred embodiment of the above composition, the polypeptide comprises SEQ ID NO:6, or an antigenic fragment or variant thereof.

In another preferred embodiment of the above composition, the polypeptide comprises SEQ ID NO:7, or an antigenic fragment or variant thereof.

In another preferred embodiment of the above composition, the polypeptide comprises SEQ ID NO:8, or an antigenic fragment or variant thereof.

In another preferred embodiment of the above composition, the polypeptide comprises SEQ ID NO:9, or an antigenic fragment or variant thereof.

In another preferred embodiment of the above composition, the polypeptide comprises SEQ ID NO:10, or an antigenic fragment or variant thereof.

In another preferred embodiment of the above composition, the polypeptide comprises SEQ ID NO:11, or an antigenic fragment or variant thereof.

In another preferred embodiment of the above composition, the polypeptide comprises SEQ ID NO:12, or an antigenic fragment or variant thereof.

In another preferred embodiment of the above composition, the polypeptide comprises SEQ ID NO:13, or an antigenic fragment or variant thereof.

In another preferred embodiment of the above composition, the polypeptide comprises SEQ ID NO:14, or an antigenic fragment or variant thereof.

In another preferred embodiment of the above composition, the polypeptide comprises SEQ ID NO:15, or an antigenic fragment or variant thereof.

In another preferred embodiment of the above composition, the polypeptide comprises SEQ ID NO:16, or an antigenic fragment or variant thereof.

In another preferred embodiment of the above composition, the polypeptide comprises SEQ ID NO:17, or an antigenic fragment or variant thereof.

In another preferred embodiment of the above composition, the polypeptide comprises SEQ ID NO:18, or an antigenic fragment or variant thereof.

In another preferred embodiment of the above composition, the polypeptide comprises SEQ ID NO:19, or an antigenic fragment or variant thereof.

In another preferred embodiment of the above composition, the polypeptide comprises SEQ ID NO:20, or an antigenic fragment or variant thereof.

In another preferred embodiment of the above composition, the polypeptide comprises SEQ ID NO:21, or an antigenic fragment or variant thereof.

In another preferred embodiment of the above composition, the polypeptide comprises SEQ ID NO:22, or an antigenic fragment or variant thereof.

In another preferred embodiment of the above composition, the polypeptide comprises SEQ ID NO:23, or an antigenic fragment or variant thereof.

In another preferred embodiment of the above composition, the polypeptide comprises SEQ ID NO:24, or an antigenic fragment or variant thereof.

In another preferred embodiment of the above composition, the polypeptide comprises SEQ ID NO:25, or an antigenic fragment or variant thereof.

In another preferred embodiment of the above composition, the polypeptide comprises SEQ ID NO:26, or an antigenic fragment or variant thereof.

In another preferred embodiment of the above composition, the polypeptide comprises SEQ ID NO:27, or an antigenic fragment or variant thereof.

In another preferred embodiment of the above composition, the polypeptide comprises SEQ ID NO:28, or an antigenic fragment or variant thereof.

In another preferred embodiment of the above composition, the polypeptide comprises SEQ ID NO:29, or an antigenic fragment or variant thereof.

In another preferred embodiment of the above composition, the polypeptide comprises SEQ ID NO:30, or an antigenic fragment or variant thereof.

In another preferred embodiment of the above composition, the polypeptide comprises SEQ ID NO:31, or an antigenic fragment or variant thereof.

In another preferred embodiment of the above composition, the polypeptide comprises SEQ ID NO:32, or an antigenic fragment or variant thereof.

In another preferred embodiment of the above composition, the polypeptide comprises SEQ ID NO:33, or an antigenic fragment or variant thereof.

In another preferred embodiment of the above composition, the polypeptide comprises SEQ ID NO:34, or an antigenic fragment or variant thereof.

In another preferred embodiment of the above composition, the polypeptide comprises SEQ ID NO:35, or an antigenic fragment or variant thereof.

In another preferred embodiment of the above composition, the polypeptide comprises SEQ ID NO:36, or an antigenic fragment or variant thereof.

In another preferred embodiment of the above composition, the polypeptide comprises SEQ ID NO:37, or an antigenic fragment or variant thereof.

In another preferred embodiment of the above composition, the polypeptide comprises SEQ ID NO:38, or an antigenic fragment or variant thereof.

In another preferred embodiment of the above composition, the polypeptide comprises SEQ ID NO:39, or an antigenic fragment or variant thereof.

In another preferred embodiment of the above composition, the polypeptide comprises SEQ ID NO:40, or an antigenic fragment or variant thereof.

In another preferred embodiment of the above composition, the polypeptide comprises SEQ ID NO:41, or an antigenic fragment or variant thereof.

In another preferred embodiment of the above composition, the polypeptide comprises SEQ ID NO:42, or an antigenic fragment or variant thereof.

In another preferred embodiment of the above composition, the polypeptide comprises SEQ ID NO:43, or an antigenic fragment or variant thereof.

In another preferred embodiment of the above composition, the polypeptide comprises SEQ ID NO:44, or an antigenic fragment or variant thereof.

In another preferred embodiment of the above composition, the polypeptide comprises SEQ ID NO:45, or an antigenic fragment or variant thereof.

In another preferred embodiment of the above composition, the polypeptide comprises SEQ ID NO:46, or an antigenic fragment or variant thereof.

In another preferred embodiment of the above composition, the polypeptide comprises SEQ ID NO:47, or an antigenic fragment or variant thereof.

In another preferred embodiment of the above composition, the polypeptide comprises SEQ ID NO:48, or an antigenic fragment or variant thereof.

In another preferred embodiment of the above composition, the polypeptide comprises SEQ ID NO:49, or an antigenic fragment or variant thereof.

In another preferred embodiment of the above composition, the polypeptide comprises SEQ ID NO:50, or an antigenic fragment or variant thereof.

In another preferred embodiment of the above composition, the polypeptide comprises SEQ ID NO:51, or an antigenic fragment or variant thereof.

In another preferred embodiment of the above composition, the polypeptide comprises SEQ ID NO:52, or an antigenic fragment or variant thereof.

In another preferred embodiment of the above composition, the polypeptide comprises SEQ ID NO:53, or an antigenic fragment or variant thereof.

In another preferred embodiment of the above composition, the polypeptide comprises SEQ ID NO:54, or an antigenic fragment or variant thereof.

In another preferred embodiment of the above composition, the polypeptide comprises SEQ ID NO:55, or an antigenic fragment or variant thereof.

In another preferred embodiment of the above composition, the polypeptide comprises SEQ ID NO:56, or an antigenic fragment or variant thereof.

In another preferred embodiment of the above composition, the polypeptide comprises SEQ ID NO:57, or an antigenic fragment or variant thereof.

In another preferred embodiment of the above composition, the polypeptide comprises SEQ ID NO:58, or an antigenic fragment or variant thereof.

In another preferred embodiment of the above composition, the polypeptide comprises SEQ ID NO:59, or an antigenic fragment or variant thereof.

In another preferred embodiment of the above composition, the polypeptide comprises SEQ ID NO:60, or an antigenic fragment or variant thereof.

In another preferred embodiment of the above composition, the polypeptide comprises SEQ ID NO:61, or an antigenic fragment or variant thereof.

In another preferred embodiment of the above composition, the polypeptide comprises SEQ ID NO:62, or an antigenic fragment or variant thereof.

In another preferred embodiment of the above composition, the polypeptide comprises SEQ ID NO:63, or an antigenic fragment or variant thereof.

In another preferred embodiment of the above composition, the polypeptide comprises SEQ ID NO:64, or an antigenic fragment or variant thereof.

In another preferred embodiment of the above composition, the polypeptide comprises SEQ ID NO:65, or an antigenic fragment or variant thereof.

In another preferred embodiment of the above composition, the polypeptide comprises SEQ ID NO:66, or an antigenic fragment or variant thereof.

In another preferred embodiment of the above composition, the polypeptide comprises SEQ ID NO:67, or an antigenic fragment or variant thereof.

In another preferred embodiment of the above composition, the polypeptide comprises SEQ ID NO:68, or an antigenic fragment or variant thereof.

In another preferred embodiment of the above composition, the polypeptide comprises SEQ ID NO:69, or an antigenic fragment or variant thereof.

In another preferred embodiment of the above composition, the polypeptide comprises SEQ ID NO:70, or an antigenic fragment or variant thereof.

In another preferred embodiment of the above composition, the polypeptide comprises SEQ ID NO:71, or an antigenic fragment or variant thereof.

In another preferred embodiment of the above composition, the polypeptide comprises SEQ ID NO:72, or an antigenic fragment or variant thereof.

In another preferred embodiment of the above composition, the polypeptide comprises SEQ ID NO:73, or an antigenic fragment or variant thereof.

In another preferred embodiment of the above composition, the polypeptide comprises SEQ ID NO:74, or an antigenic fragment or variant thereof.

In another preferred embodiment of the above composition, the polypeptide comprises SEQ ID NO:75, or an antigenic fragment or variant thereof.

In another preferred embodiment of the above composition, the polypeptide comprises SEQ ID NO:76, or an antigenic fragment or variant thereof.

In another preferred embodiment of the above composition, the polypeptide comprises SEQ ID NO:77, or an antigenic fragment or variant thereof.

In another preferred embodiment of the above composition, the polypeptide comprises SEQ ID NO:78, or an antigenic fragment or variant thereof.

In another preferred embodiment of the above composition, the polypeptide comprises SEQ ID NO:79, or an antigenic fragment or variant thereof.

In another preferred embodiment of the above composition, the polypeptide comprises SEQ ID NO:80, or an antigenic fragment or variant thereof.

In another preferred embodiment of the above composition, the polypeptide comprises SEQ ID NO:81, or an antigenic fragment or variant thereof.

In another preferred embodiment of the above composition, the polypeptide comprises SEQ ID NO:82, or an antigenic fragment or variant thereof.

In another preferred embodiment of the above composition, the polypeptide comprises SEQ ID NO:83, or an antigenic fragment or variant thereof.

In another preferred embodiment of the above composition, the polypeptide comprises SEQ ID NO:84, or an antigenic fragment or variant thereof.

In another preferred embodiment of the above composition, the polypeptide comprises SEQ ID NO:85, or an antigenic fragment or variant thereof.

In another preferred embodiment of the above composition, the polypeptide comprises SEQ ID NO:86, or an antigenic fragment or variant thereof.

In another preferred embodiment of the above composition, the polypeptide comprises SEQ ID NO:87, or an antigenic fragment or variant thereof.

In another preferred embodiment of the above composition, the polypeptide comprises SEQ ID NO:88, or an antigenic fragment or variant thereof.

In another preferred embodiment of the above composition, the polypeptide comprises SEQ ID NO:89, or an antigenic fragment or variant thereof.

In another preferred embodiment of the above composition, the polypeptide comprises SEQ ID NO:90, or an antigenic fragment or variant thereof.

In another preferred embodiment of the above composition, the polypeptide comprises SEQ ID NO:91, or an antigenic fragment or variant thereof.

In another preferred embodiment of the above composition, the polypeptide comprises SEQ ID NO:92, or an antigenic fragment or variant thereof.

In another preferred embodiment of the above composition, the polypeptide comprises SEQ ID NO:93, or an antigenic fragment or variant thereof.

In another preferred embodiment of the above composition, the polypeptide comprises SEQ ID NO:94, or an antigenic fragment or variant thereof.

In another preferred embodiment of the above composition, the polypeptide comprises SEQ ID NO:95, or an antigenic fragment or variant thereof.

In another preferred embodiment of the above composition, the polypeptide comprises SEQ ID NO:96, or an antigenic fragment or variant thereof.

In another, preferred embodiment of the above composition, the polypeptide comprises SEQ ID NO:97, or an antigenic fragment or variant thereof.

In another preferred embodiment of the above composition, the polypeptide comprises SEQ ID NO:98, or an antigenic fragment or variant thereof.

In another preferred embodiment of the above composition, the polypeptide comprises SEQ ID NO:99, or an antigenic fragment or variant thereof.

In another preferred embodiment of the above composition, the polypeptide comprises SEQ ID NO:100, or an antigenic fragment or variant thereof.

In another preferred embodiment of the above composition, the polypeptide comprises SEQ ID NO:101, or an antigenic fragment or variant thereof.

In another preferred embodiment of the above composition, the polypeptide comprises SEQ ID NO:102, or an antigenic fragment or variant thereof.

In another preferred embodiment of the above composition, the polypeptide comprises SEQ ID NO:103, or an antigenic fragment or variant thereof.

In another preferred embodiment of the above composition, the polypeptide comprises SEQ ID NO:104, or an antigenic fragment or variant thereof.

In another preferred embodiment of the above composition, the polypeptide comprises SEQ ID NO:105, or an antigenic fragment or variant thereof.

In another preferred embodiment of the above composition, the polypeptide comprises SEQ ID NO:168, or an antigenic fragment or variant thereof.

A composition comprising the polypeptide of SEQ ID NO:16, or an antigenic fragment or variant thereof for use as a medicament is at present the most preferred embodiment.

In some embodiments of the composition, the polypeptide consists of a sequence selected from the group of SEQ ID NO:1-282. In other embodiments, the polypeptide comprises a sequence selected from the group of SEQ ID NO:1-282 or an antigenic fragment or variant of said sequence, as well as a tag, such as a his-tag, i.e. a polyhistidine tag.

In another preferred embodiment, the polypeptide in the composition of the invention may be combined with or fused to a toxin, e.g. an enterotoxigenic Escherichia coli Stable or Labile toxin. A suitable heat stable toxin II (STII) has been described in Lee et al. (1983) Infect. Immun. 42: 264-268. Examples of suitable fusion proteins are given in SEQ ID NO:295 and SEQ ID NO:296. In one embodiment, the combination comprises the polypeptide of the invention and a non-covalently linked toxin, wherein the toxin may be a single toxin polypeptide, or a multimeric, e.g. dimeric, form comprising multiple copies of the toxin. In another embodiment, the polypeptide of the invention and the toxin are covalently linked, e.g. by post-translational linkage or transcription and translation from a single fused open reading frame. In either case, the two constituents may be linked directly or via a spacer or linker domain, which e.g. may be a peptide linker, preferably a protease-resistant and/or non-immunogenic peptide linker. Such peptide linker may be of any length, e.g. it may be between 2 and 200, such as between 5 and 50 amino acids in length. Multiple copies of the toxin may be fused to the polypeptide of the invention.

A composition comprising a polypeptide of the invention, e.g. the polypeptide of SEQ ID NO:16, as well as an enterotoxigenic Escherichia coli may be used to manufacture a vaccine for prevention of infection with Streptococcus pneumoniae and/or enterotoxigenic Escherichia.

In further embodiments, the composition of the invention may comprise dimers of any of the polypeptides of SEQ ID NO:1-282, such as dimers of SEQ ID NO:16. Dimers may e.g. be formed by post-translational linkage or be generated from a single fused open reading frame. In either case, the two monomer units of the dimer may be linked directly or via a spacer or linker domain, which e.g. may be a peptide linker, preferably a protease-resistant and/or non-immunogenic peptide linker. Such a peptide linker may be of any length, e.g. it may be between 2 and 200, such as between 5 and 50 amino acids in length.

The composition may only comprise one polypeptide selected from the group of SEQ ID NO:1-282 or a antigenic fragment or variant thereof. However, in other embodiments, the composition comprises more than one polypeptide of the group of SEQ ID NO:1-282 and/or more than one antigenic fragment of a polypeptide selected from the group of SEQ ID NO:1-282. Thus, the composition according to the invention may comprise more than one, such as 2, for example 3, such as 4, for example 5, such as 6, for example 7, such as 8, for example 9, such as 10, such as a number of polypeptides and/or fragments in the range of from 5 to 10, or more than 10, such as for example in the range of from 10 to 20, different polypeptides selected from the group of SEQ ID NO:1-282 or antigenic fragments or variants thereof.

Similarly, the composition may only comprise one polynucleotide, one expression vector or one recombinant virus or recombinant cell of the invention. However, in other embodiments, the composition comprises more than one polynucleotide, one expression vector or one recombinant virus or recombinant cell of the invention. Thus, the composition according to the invention may comprise more than one, such as 2, for example 3, such as 4, for example 5, such as 6, for example 7, such as 8, for example 9, such as 10, or more than 10, such as for example in the range of from 10 to 20, different polynucleotides, expression vectors or recombinant viruses or recombinant cells of the invention as described herein.

Furthermore, in some embodiments, a recombinant cell of the invention may express more than one polypeptide of the group of SEQ ID NO:1-282 and/or more than one antigenic fragment or variant of a polypeptide selected from the group of SEQ ID NO:1-282. Thus, the composition according to the invention may comprise a recombinant cell comprising more than one, such as 2, for example 3, such as 4, for example 5, such as 6, for example 7, such as 8, for example 9, such as 10, such as a number of polypeptides and/or antigenic fragments or variants in the range of from 5 to 10, or more than 10, such as for example in the range of from 10 to 20, different polypeptides selected from the group of SEQ ID NO:1-282 or antigenic fragments or variants thereof. In another embodiment, the composition for use in the invention comprises multiple of the recombinant viruses or recombinant cells described herein.

Preferably, the composition of the invention comprises one of the combinations of polypeptides (or antigenic fragments or variants thereof) given in Table 1.

In Table 1, each of the crosses (“x”) placed at the crossing of a column designated with a SEQ ID number with a row designated by another SEQ ID number indicates a composition comprising the two polypeptides of those two SEQ ID numbers (or anti-genic fragments or variants thereof).

I.e. as an example, entirely for illustrative purposes and not intended in a limiting manner, the cross (“x”) at the crossing of the column of SEQ ID NO:2 (“2”) with the row of SEQ ID NO:1 (“1”) indicates a composition comprising:
the polypeptide of SEQ ID NO: 1 or an antigenic fragment or variant thereof and
the polypeptide of SEQ ID NO:2 or an antigenic fragment or variant thereof.

Highly preferred compositions include:

A composition comprising:
the polypeptide of SEQ ID NO:16 or an antigenic fragment or variant thereof and
any of the polypeptides of SEQ ID NO:1-282 or an antigenic fragment or variant thereof, preferably any of SEQ ID NO:1-41 or an antigenic fragment or variant thereof, more preferably a polypeptide selected from the group consisting of SEQ ID NO:10, SEQ ID NO:13, SEQ ID NO:20 and SEQ ID NO:28, most preferably the polypeptide of SEQ ID NO:20 or an antigenic fragment or variant thereof.

A composition comprising:
the polypeptide of SEQ ID NO:10 or an antigenic fragment or variant thereof and
any of the polypeptides of SEQ ID NO:1-282 or an antigenic fragment or variant thereof, preferably any of SEQ ID NO:1-41 or an antigenic fragment or variant thereof, more preferably a polypeptide selected from the group consisting of SEQ ID NO:13, SEQ ID NO:20 and SEQ DI NO:28, most preferably the polypeptide of SEQ ID NO:20 or an antigenic fragment or variant thereof.

A composition comprising:
the polypeptide of SEQ ID NO:13 or an antigenic fragment or variant thereof and
any of the polypeptides of SEQ ID NO:1-282 or an antigenic fragment or variant thereof, preferably any of SEQ ID NO:1-41 or an antigenic fragment or variant thereof, more preferably a polypeptide selected from the group consisting of SEQ ID NO:20 and SEQ ID NO:28, most preferably the polypeptide of SEQ ID NO:20 or an antigenic fragment or variant thereof.

A composition comprising:
the polypeptide of SEQ ID NO:28 or an antigenic fragment or variant thereof and
any of the polypeptides of SEQ ID NO:1-282 or an antigenic fragment or variant thereof, preferably any of SEQ ID NO:1-41 or an antigenic fragment or variant thereof, the polypeptide of SEQ ID NO:20 or an antigenic fragment or variant thereof.

Preferred compositions comprising at least three polypeptides include the following:

A composition comprising three or more polypeptides selected from the group consisting of SEQ ID NO:10, SEQ ID NO:13, SEQ ID NO:16, SEQ ID NO:20 and SEQ ID NO:28.

A composition comprising:
the polypeptide of SEQ ID NO:16 or an antigenic fragment or variant thereof
   and
the polypeptide of SEQ ID NO:20 or an antigenic fragment or variant thereof
   and
any of the polypeptides of SEQ ID NO:1-282 or an antigenic fragment or variant thereof, preferably any of SEQ ID NO:1-41 or an antigenic fragment or variant thereof, more preferably a polypeptide selected from the group consisting of SEQ ID NO:10, SEQ ID NO:13 and SEQ ID NO:28.

Further preferred compositions according to the invention, comprising four or more polypeptides selected from the group consisting of SEQ ID NO:10, SEQ ID NO:13, SEQ ID NO:16, SEQ ID NO:20 and SEQ ID NO. 28.

In a yet further preferred embodiment, the composition of the invention comprises the five polypeptides of SEQ ID NO:10, SEQ ID NO:13, SEQ ID NO:16, SEQ ID NO:20 and SEQ ID NO. 28.

In some embodiments of the above compositions comprising two or more polypeptides, the polypeptides are not covalently linked. In other embodiments, however, the polypeptides may form a fusion polypeptide, which is formed by post-translational linkage or generated from a single fused open reading frame. In either case, the two or more polypeptides may be linked directly or via a spacer or linker domain, which e.g. may be a peptide linker, preferably a protease-resistant and/or non-immunogenic peptide linker. Such a peptide linker may be of any length, e.g. it may be between 2 and 200, such as between 5 and 50 amino acids in length.

### Vaccines Comprising Polypeptides

As described above, in a preferred embodiment, the invention relates to a composition comprising a polypeptide which comprises a sequence selected from the group consisting of SEQ ID NO:1-282, or an antigenic fragment or variant of said sequence, for use as a vaccine. Preferred fragments and variants are those described in the sections herein that relate to fragments and variants.

Accordingly, in this embodiment, the antigenicity or immunogenicity is provided by direct administration of a polypeptide normally located on the surface of a Streptococcus pneumoniae cell. In one particular embodiment, the polypeptides are selected so that the vaccine composition comprises multiple polypeptides capable of associating with different MHC molecules, such as different MHC class I molecules. Preferably, the composition for use as a vaccine comprises polypeptides and/or fragments capable of associating with the most frequently occurring MHC class I molecules. In one particular embodiment of the invention, the composition comprises one or more polypeptides and/or fragments capable of associating to an MHC class I molecule and one or more polypeptides and/or fragments capable of associating with an MHC class II molecule. Hence, the vaccine composition is in some embodiments capable of raising a specific cytotoxic T-cells response and/or a specific helper T-cell response. Association to MHC molecules can e.g. be determined as described by Andersen et al. (1999) Tissue Antigens 54:185; or by Tan et al. (1997) J. Immunol. Methods 209:25.

### Adjuvants and Immunogenic Carriers

Preferably, the composition for use as vaccine, i.e. the vaccine composition, of the present invention comprises a pharmaceutically-acceptable carrier as described herein in the section ‘Compositions for use in the invention’.

The composition can further comprise an adjuvant. Adjuvants are substances whose admixture into the vaccine composition increases or otherwise modifies the immune response to a polypeptide or other antigen. Adjuvants could for example be any of: AlK(SO₄)₂, AlNa(SO₄)₂, AlNH₄ (SO₄), silica, alum, Al(OH)₃, Ca₃ (PO₄)₂, kaolin, carbon, aluminium hydroxide, aluminium phosphate, muramyl dipeptides, N-acetyl-muramyl-L-threonyl-D-isoglutamine (thr-DMP), N-acetyl-nornuramyl-Lalanyl-D-iso-glutamine (CGP 11687, also referred to as nor-MDP), N-acetylmuramyl-L-alanyl-D-isoglutaminyl-L-alanine-2-(1′2′-dipalmitoyl-sn-glycero-3-hydroxphosphoryl oxy)-ethylamine (CGP 19835A, also referred to as MTP-PE), RIBI (MPL+TDM+CWS) in a 2% squalene/Tween-80® emulsion, lipopolysaccharides and derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, liposomes or other lipid emulsions, Titermax, ISCOMS, Quil A, ALUN (see U.S. Pat. Nos. 58,767 and 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Preferred adjuvants to be used with the invention include alum, Montanide ISA-51 and QS-21. Montanide ISA-51 (Seppic, Inc.) is a mineral oil-based adjuvant analogous to incomplete Freund's adjuvant, which is normally administered as an emulsion. QS-21 (Antigenics; Aquila Biopharmaceuticals, Framingham, Mass.) is a highly purified, water-soluble saponin that handles as an aqueous solution. Another preferred adjuvant to be used in the composition of the invention is IMSAVAC-L from the Netherlands Vaccine Institute. In another preferred embodiment, the polypeptide or polypeptides are included in virosomes.

Desirable functionalities of adjuvants capable of being used in accordance with the present invention are listed in the below table.

TABLE 1
Modes of adjuvant action

| Action | Adjuvant type | Benefit |
|---|---|---|
| 1. Immunomodulation | Generally small molecules or proteins which modify the | Upregulation of immune response. Selection of Th1 or |
| | cytokine network | Th2 |
| 2. Presentation | Generally amphipathic molecules or complexes which | Increased neutralizing antibody response. Greater |
| | interact with immunogen in its native conformation | duration of response |
| 3. CTL Induction | Particles which can bind or enclose immunogen and | Cytosolic processing of protein yielding correct class |
| | which can fuse with or disrupt cell membranes | 1 restricted peptides |
| | w/o emulsions for direct attachment of peptide to cell | Simple process if promiscuous peptide(s) known |
| | surface MHC-1 | |
| 4. Targeting | Particulate adjuvants which bind immunogen. Adjuvants | Efficient use of adjuvant and immunogen |
| | which saturate Kupffer cells | |
| | Carbohydrate adjuvants which target lectin receptors on | As above. May also determine type of response if |
| | macrophages and DCs | targeting selective |
| 5. Depot generation | w/o emulsion for short term | Efficiency |
| | Microspheres or nanospheres for long term | Potential for single-dose vaccine |

Source: John C. Cox and Alan R. Coulter Vaccine 1997 February; 15(3):248-56
   A vaccine composition according to the present invention may comprise more than one different adjuvant. It is also contemplated that the Streptococcus pneumoniae polypeptide of the invention, or one or more antigenic fragments thereof, and the adjuvant can be administered separately in any appropriate sequence.

The adjuvant of choice may e.g. Freund's complete or incomplete adjuvant, or killed B. pertussis organisms, used e.g. in combination with alum precipitated antigen. A general discussion of adjuvants is provided in Goding, Monoclonal Antibodies: Principles & Practice (2nd edition, 1986) at pages 61-63. Goding notes, however, that when the antigen of interest is of low molecular weight, or is poorly immunogenic, coupling to an immunogenic carrier is recommended (see below). Various saponin extracts and cytokines have also been suggested to be useful as adjuvants in immunogenic compositions. Recently, it has been proposed to use granulocyte-macrophage colony stimulating factor (GM-CSF), a well known cytokine, as an adjuvant (WO 97/28816).

In addition, a vaccine composition of the invention can comprise an immunogenic carrier such as a scaffold structure, for example a protein or a polysaccharide, to which the Streptococcus pneumoniae polypeptide or the fragment thereof is capable of being associated. A Streptococcus pneumoniae polypeptide, or the antigenic fragment or variant thereof, present in the vaccine composition can thus be associated with an immunogenic carrier such as e.g. a protein. The binding or association of the polypeptide to a carrier protein may be covalent or non-covalent. An immunogenic carrier protein may be present independently of an adjuvant. The function of a carrier protein can for example be to increase the molecular weight of in particular fragments in order to increase their activity or immunogenicity, to confer stability, to increase the biological activity, or to increase serum half-life. Furthermore, an immunogenic carrier protein may aid presenting the Streptococcus pneumoniae polypeptide or the fragments thereof to T cells. A carrier protein could be, but is not limited to, keyhole limpet hemocyanin, serum proteins such as transferrin, bovine serum albumin, human serum albumin, thyroglobulin or ovalbumin, Immunoglobulins, or hormones, such as insulin. Tetanus toxoid and/or diphtheria toxoid are also suitable carriers in one embodiment of the invention. Alternatively or additionally, dextrans, for example sepharose may be added. In yet another embodiment, an antigen-presenting cell such as e.g. a dendritic cell capable of presenting the polypeptide or a fragment thereof to a T cell may be added to obtain the same effect as a carrier protein. Methods for the preparation of vaccine compositions have e.g. been described in U.S. Pat. No. 5,470,958 and references therein.

In a further embodiment, the vaccine composition of the invention may comprise Streptococcus pneumoniae carbohydrates in addition to a polypeptide of the invention. In one embodiment, the added carbohydrates are carbohydrates derived from or characteristic of one or more serotypes of Streptococcus pneumoniae. In a preferred embodiment, the polypeptide of the invention is combined with polysaccharides derived from or characteristic of any one or more of the serotypes given in Table 4. In a preferred embodiment, the polypeptide is combined with one or more, preferably two, three, four, five, six or seven polysaccharides derived from or characteristic of serotype 4, 6B, 9V, 14, 18C, 19F and 23F. In another embodiment, the polypeptide is combined with eight or more, preferably ten or more, 15 or more, or 20 or more of the polysaccharide antigens of serotypes 1, 2, 3, 4, 5, 6B, 7F, 8, 9N, 10A, 11A, 12F, 14, 15B, 17F, 18C, 19A, 19F, 20, 22F, 23F, and 33F. These carbohydrates may added in free form to the vaccine composition of the invention, or, alternatively, they may be fused to a polypeptide of the invention to be used in the vaccine composition.

An effective amount of a polypeptide of the invention may be an amount capable of eliciting a detectable humoral immune response in the absence of an immunomodulator. The appropriate amount of immunogen to be used is dependent on the immunological response it is desired to elicit. Furthermore, the exact effective amount necessary may vary from subject to subject, depending on the species, age and general condition of the subject, the severity of the condition being treated, the mode of administration, etc. The polypeptide vaccines of the present invention may be administered in various dosages, including dosages that are lower than those normally used for other vaccines. This possible because the polypeptides of the present invention are abundant on the surface of a Streptococcus pneumoniae cell and thus even a fairly low level of response can provide immunity. Thus, dosage of a polypeptide of the invention, when used for immunisation, may e.g. be from 0.1 to 500 micrograms per kilogram body weight, such as from 0.1 to 100 micrograms, e.g. from 0.1 to 50 micrograms, such as from 0.1 to 25 micrograms, such as in the range of from 8 to 25 micrograms per kilogram body weight, or less than that, such as from 0.1 to 5 micrograms or from 0.1 to 2 micrograms per kilograms body weight.

### DNA Vaccine Compositions and Vaccine Compositions Comprising Recombinant Viruses or Recombinant Cells

DNA or RNA vaccines pertain to the introduction of e.g. an antigenic polypeptide determinant into a patient by overexpressing in the cells of the patient, a polynucleotide construct which includes expression control sequences operably linked to a sequence encoding the polypeptide of interest, herein a polypeptide of any of SEQ ID NO:1-282 or an antigenic fragment or variant thereof, preferably the polypeptide of SEQ ID NO:16 or an antigenic fragment or variant thereof. As such fragments may not contain a methionine start codon, such a codon is optionally included as part of the expression control sequences. The polynucleotide construct may be a non-replicating and linear polynucleotide, a circular expression vector, or an autonomously replicating plasmid or viral expression vector. The construct may become integrated into the host genome. Any expression vector that can transfect a mammalian cell may be used in the methods of immunising an individual according to the present invention. Methods for constructing expression vectors are well known in the art (see, e.g., Molecular Cloning: A Laboratory Manual, Sambrook et al., eds., Cold Spring Harbor Laboratory, 2nd Edition, Cold Spring Harbor, N.Y., 1989). Preferred are compositions comprising a plurality of genes expressing multiple polypeptides selected from SEQ ID NO:1-282 and/or multiple antigenic fragments of the invention, thereby permitting simultaneous vaccination using a variety of preselected targets.

Vaccines can also be prepared by incorporating a polynucleotide encoding a specific antigenic polypeptide of interest into a living but harmless vector, such as a virus or a cell, such as an attenuated or reduced-virulence E. coli or Salmonella cell. The harmless recombinant virus or recombinant cell is injected into the intended recipient. Such a recombinant cell may be dead or alive. If alive, the recombinant organism may replicate in the host while producing and presenting the antigenic polypeptide to the host's immune system. It is contemplated that this type of vaccine may be more effective than the non-replicative type of vaccine. For such a vaccine to be successful, the vector organism must be viable, and either be naturally non-virulent or have an attenuated or reduced-virulence phenotype.

Strategies for vaccination using attenuated bacteria and suitable bacterial strains for use therein have been described in e.g. Makino et al. (2001) Microb. Pathog. 31:1-8; Gentschev et al. (2002) Int. J. Med. Microbiol. 291:577-582; Turner et al. (2001) Infect. Immun. 69:4969-4979; WO99/49026; and WO03/022307.

Further examples of vectors that can be applied are vectors comprising e.g., retroviruses, as disclosed in WO 90/07936, WO 91/02805, WO 93/25234, WO 93/25698, and WO 94/03622, adenovirus, as disclosed by Berkner, Biotechniques 6:616-627, 1988; Li et al., Hum. Gene Ther. 4:403-409, 1993; Vincent et al., Nat. Genet. 5:130-134, 1993; and Kolls et al., Proc. Natl. Acad. Sci. USA 91:215-219, 1994), pox virus, as disclosed by U.S. Pat. No. 4,769,330; U.S. Pat. No. 5,017,487; and WO 89/01973, naked DNA as disclosed WO 90/11092, a polynucleotide molecule complexed to a polycationic molecule as disclosed in WO 93/03709, and polynucleotides associated with liposomes as disclosed by Wang et al., Proc. Natl. Acad. Sci. USA 84:7851, 1987. In certain embodiments, the DNA may be linked to killed or inactivated adenovirus as disclosed by Curiel et al., Hum. Gene Ther. 3:147-154, 1992; Cotton et al., Proc. Natl. Acad. Sci. USA 89:6094, 1992. Other suitable compositions include DNA-ligands as disclosed by Wu et al., J. Biol. Chem. 264:16985-16987, 1989), and lipid-DNA combinations as disclosed by Felgner et al., Proc. Natl. Acad. Sci. USA 84:7413-7417, 1989). In addition, the efficiency of naked DNA uptake into cells may be increased by coating the DNA onto biodegradable latex beads.

Vaccine vectors preferably comprise a suitable promoter which is operably linked to the polynucleotide sequence encoding the immunogenic polypeptide. Any promoter that can direct a high level of transcription initiation in the target cells may be used in the invention. Non-tissue specific promoters, such as the cytomegalovirus (DeBemardi et al., Proc Natl Acad Sci USA 88:9257-9261 [1991], and references therein), mouse metallothionine I (Hammer et al., J Mol Appl Gen 1:273-288 [1982]), HSV thymidine kinase (McKnight, Cell 31:355-365 [1982]), and SV40 early (Benoist et al., Nature 290:304-310 [1981]) promoters may thus also be used.

### Methods of Vaccination and Use for Vaccination/Immunisation

In a further main aspect, the present invention relates to the use of a composition comprising any one or more of
a polypeptide which comprises a sequence selected from the group consisting of SEQ ID NO:1-282, or comprises an antigenic fragment or variant of said sequence,
a polynucleotide comprising a sequence encoding said polypeptide,
an expression vector comprising a sequence encoding said polypeptide, or
a recombinant virus or recombinant cell comprising said polynucleotide or said expression vector,
   for the preparation of a medicament for the immunisation of an animal or human being against bacterial infections. The immunisation preferably induces a protective immune response. In one embodiment of the above use, the medicament is only given once.

In a preferred embodiment, the medicament is for the immunisation against Streptococcus infections. Most preferably, the medicament is for immunisation against Streptococcus pneumoniae. Immunisation with a Streptococcus pneumoniae polypeptide can, however, also give cross-protection to other bacterial species. This normally requires significant homology to at least a portion of a polypeptide of the other species. Such homology is e.g. found between SEQ ID NO:16 and variants thereof from Streptococcus pyogenes (group A Streptococcus) (SEQ ID NO:283), Streptococcus agalactiae (group B Streptococcus) (SEQ ID NO:284) and Listeria monocytogenes (SEQ ID NO:285). Similarly, homology is found between SEQ ID NO:20 and variants thereof from Streptococcus pyogenes (group A Streptococcus) (SEQ ID NO:286), Streptococcus agalactiae (group B Streptococcus) (SEQ ID NO:287) and Listeria monocytogenes (SEQ ID NO:288).

Accordingly, the medicament is in some embodiments used for the immunisation against one or more of: Streptococcus pyogenes (group A Streptococcus), Streptococcus agalactiae (group B Streptococcus) and Listeria monocytogenes. Highly preferred polypeptides for use in the preparation of such a medicament are SEQ ID NO:16 and SEQ ID NO:20.

An alternative strategy for immunisation against one or more bacteria is to immunise with a medicament comprising the variant polypeptide. Accordingly, in a further embodiment, the polypeptide used for the preparation of the medicament is a variant of any of SEQ ID NO:1-282, preferably a variant of SEQ ID NO:16 and/or a variant of SEQ ID NO:20. Most preferably, the polypeptide is selected from the group consisting of SEQ ID NO:283, SEQ ID NO:284, SEQ ID NO:285, SEQ ID NO:286, SEQ ID NO:287 and SEQ ID NO:288, or a fragment thereof or a variant thereof, e.g. a variant having more than 95%, such as more than 98% sequence identity to SEQ ID NO:283, SEQ ID NO:284, SEQ ID NO:285, SEQ ID NO:286, SEQ ID NO:287 or SEQ ID NO:288.

Accordingly, in some embodiments:
a medicament comprising SEQ ID NO:283 and/or SEQ ID NO:286, or a fragment or variant of any of these two, is used to immunise against Streptococcus pyogenes and/or Streptococcus pneumoniae and/or other bacteria;
a medicament comprising SEQ ID NO:284 and/or SEQ ID NO:287, or a fragment or a variant of any of these two, is used to immunise against Streptococcus agalactiae and/or Streptococcus pneumoniae and/or other bacteria
   or
a medicament comprising SEQ ID NO:285 and/or SEQ ID NO:288, or a fragment or a variant of any of these two, is used immunise against Listeria monocytogenes and/or Streptococcus pneumoniae and/or other bacteria

In the most preferred embodiment, the composition herein comprises or further comprises
a polypeptide which comprises SEQ ID NO:16, or comprises an antigenic fragment or variant of SEQ ID NO:16,
a polynucleotide comprising a sequence encoding said polypeptide,
an expression vector comprising a sequence encoding said polypeptide, or
a recombinant virus or recombinant cell comprising said polynucleotide or said expression vector.

In another preferred embodiment, the composition herein comprises or further comprises
a polypeptide which comprises SEQ ID NO:10, or comprises an antigenic fragment or variant of SEQ ID NO:10,
a polynucleotide comprising a sequence encoding said polypeptide,
an expression vector comprising a sequence encoding said polypeptide, or a recombinant virus or recombinant cell comprising said polynucleotide or said expression vector.

In another preferred embodiment, the composition herein comprises or further comprises
a polypeptide which comprises SEQ ID NO:13, or comprises an antigenic fragment or variant of SEQ ID NO:13,
a polynucleotide comprising a sequence encoding said polypeptide,
an expression vector comprising a sequence encoding said polypeptide, or
a recombinant virus or recombinant cell comprising said polynucleotide or said expression vector.

In another preferred embodiment, the composition herein comprises or further comprises
a polypeptide which comprises SEQ ID NO:28, or comprises an antigenic fragment or variant of SEQ ID NO:28,
a polynucleotide comprising a sequence encoding said polypeptide,
an expression vector comprising a sequence encoding said polypeptide, or
a recombinant virus or recombinant cell comprising said polynucleotide or said expression vector.

In another preferred embodiment, the composition further comprises
a polypeptide which comprises SEQ ID NO:20, or comprises an antigenic fragment or variant of SEQ ID NO:20,
a polynucleotide comprising a sequence encoding said polypeptide,
an expression vector comprising a sequence encoding said polypeptide, or
a recombinant virus or recombinant cell comprising said polynucleotide or said expression vector.

Similarly, the invention relates to a method for the immunisation of an animal or human being against a Streptococcus pneumoniae infections comprising the step of administering any one or more of
a polypeptide which comprises any of the sequences of SEQ ID NO:1-282, or comprises a fragment or variant of any of said sequences,
a polynucleotide comprising a sequence encoding said polypeptide,
an expression vector comprising a sequence encoding said polypeptide, or
a recombinant virus or recombinant cell comprising said polynucleotide or said expression vector,
   thereby immunising said animal or human being against Streptococcus pneumoniae infections.

In one embodiment of the above method for immunisation, said
polypeptide which comprises any of the sequences of SEQ ID NO:1-282, preferably SEQ ID NO:16, or comprises a fragment or variant of any of said sequences,
polynucleotide comprising a sequence encoding said polypeptide,
expression vector comprising a sequence encoding said polypeptide, or
recombinant virus or recombinant cell comprising said polynucleotide or said expression vector,
   is only given once, thereby immunising said animal or human being against Streptococcus pneumoniae infections through a single administration.
   The animal may be any bird or mammal, e.g. a chicken, duck, turkey, cow or pig. Particular target populations of human beings may be individuals from at-risk populations, such as the population of children up to 4 years old, the population of elderly persons or the population of naive or semi-immune travelers to the developing world.

Because the polypeptides of the present invention are immunogenic and because they are abundant on the Streptococcus pneumoniae cell, a protective immune response can be induced even patients with a reduced ability to respond to antigenic stimuli, such as juveniles, elderly patients or immunocompromised patients. Furthermore, for the same reasons, the vaccines of the invention can also be used to prevent otitis media, to prevent nasopharyngal carriage of Streptococcus pneumoniae, to prevent sepsis caused by Streptococcus pneumoniae, or to prevent meningitis caused by Streptococcus pneumoniae.

Thus, in one embodiment, the present invention relates to the use of any one or more of
a polypeptide which comprises a sequence selected from the group consisting of SEQ ID NO:1-282, preferably SEQ ID NO:16, or comprises an antigenic fragment or variant of said sequence,
a polynucleotide comprising a sequence encoding said polypeptide,
an expression vector comprising a sequence encoding said polypeptide, or
a recombinant virus or recombinant cell comprising said polynucleotide or said expression vector,
   for the preparation of a medicament for the immunisation of an animal or human being against Streptococcus pneumoniae infections, wherein said human being is a child of less than 4 yours of age, such as less than 2 years of age, e.g. less than 1 year of age, and/or a child having maternal immunity (i.e. having maternal antibodies in circulation).

In a further embodiment, the present invention relates to the use of any one or more of
a polypeptide which comprises a sequence selected from the group consisting of SEQ ID NO:1-282, preferably SEQ ID NO:16, or comprises an antigenic fragment or variant of said sequence,
a polynucleotide comprising a sequence encoding said polypeptide,
an expression vector comprising a sequence encoding said polypeptide, or
a recombinant virus or recombinant cell comprising said polynucleotide or said expression vector,
   for the preparation of a medicament for the immunisation of an animal or human being against Streptococcus pneumoniae infections, wherein said human being is an immunocompromised patient. Immunocompromised patients could e.g. patients taking immunosuppressive chemotherapy or patients with congenital or acquired immune deficiency. For the immunisation to be effective in these patients, it is required that the patient still to some extent is capable of producing an immune response.

In another embodiment, the present invention relates to the use of any one or more of
a polypeptide which comprises a sequence selected from the group consisting of SEQ ID NO:1-282, preferably SEQ ID NO:16, or comprises an antigenic fragment or variant of said sequence,
a polynucleotide comprising a sequence encoding said polypeptide,
an expression vector comprising a sequence encoding said polypeptide, or a recombinant virus or recombinant cell comprising said polynucleotide or said expression vector,
   for the preparation of a medicament for the prevention of otitis media, in particular otitis media due to Streptococcus pneumoniae.

In yet another embodiment, the present invention relates to the use of any one or more of
a polypeptide which comprises a sequence selected from the group consisting of SEQ ID NO:1-282, preferably SEQ ID NO:16, or comprises an antigenic fragment or variant of said sequence,
a polynucleotide comprising a sequence encoding said polypeptide,
an expression vector comprising a sequence encoding said polypeptide, or
a recombinant virus or recombinant cell comprising said polynucleotide or said expression vector,
   for the preparation of a medicament for the treatment and/or prevention of nasopharyngal carriage of Streptococcus pneumoniae.

In an even further embodiment, the present invention relates to the use of any one or more of
a polypeptide which comprises a sequence selected from the group consisting of SEQ ID NO:1-282, preferably SEQ ID NO:16, or comprises an antigenic fragment or variant of said sequence,
a polynucleotide comprising a sequence encoding said polypeptide,
an expression vector comprising a sequence encoding said polypeptide, or
a recombinant virus or recombinant cell comprising said polynucleotide or said expression vector,
   for the preparation of a medicament for the prevention of Streptococcal meningitis.

The vaccines may be administered in the dosages described herein by any suitable mode of administration, including modes of administration that result in a less than complete (e.g. less than 50% or less than 90%) uptake of all administered antigen. This is possible because the polypeptides of the present invention are sufficiently immunogenic and because, due to their abundance on the surface of the Streptococcus pneumoniae cell, even a somewhat suboptimal response, can provide immunity. Thus, modes of administration of the composition according to the invention include, but are not limited to systemic administration, such as intravenous or subcutaneous administration, transdermal administration, intradermal administration, intramuscular administration, intranasal administration, oral administration, and generally any form of mucosal administration.

An important problem relating to the production of effective Streptococcus vaccines is the occurrence of immunologically different types, also termed serotypes, of the bacteria. These types differ considerably in their polysaccharide profile and also, albeit less, in some highly variable proteins. Due to such variability, vaccines known in the art often only work against some and not all serotypes.

The vaccines of the present invention are based on abundant surface-located polypeptides, which are not highly variable. These vaccines will be effective against a plurality of serotypes. Accordingly, in one embodiment, the invention relates to the use of any one or more of
a polypeptide which comprises a sequence selected from the group consisting of SEQ ID NO:1-282, preferably selected from the group consisting of SEQ ID NO:1-41, most preferably SEQ ID NO:16, or comprises an antigenic fragment or variant of said sequence,
a polynucleotide comprising a sequence encoding said polypeptide,
an expression vector comprising a sequence encoding said polypeptide, or
a recombinant virus or recombinant cell comprising said polynucleotide or said expression vector,
   for the preparation of a medicament for the immunisation of an animal or human being against more than one serotype of Streptococcus pneumoniae, such as 5 or more different serotypes, e.g. 8 or more different serotypes, such as 15 or more different serotypes, e.g. 24 or more different serotypes.

Preferably, said more than one serotype includes a serotype selected from the group of 6A, 7C, 9A, 10B, 13, 15C, 16F, 18B, 21, 23A, 24F, 28F, 31, 34, 35F, 35B, 38.

In one preferred embodiment, the medicament is used for the immunisation against at least the serotypes 4, 6B, 9V, 14, 18C, 19F and 23F, and, preferably, furthermore at least one further serotype, said further serotype preferably being selected from the group of 6A, 7C, 9A, 10B, 13, 15C, 16F, 18B, 21, 23A, 24F, 28F, 31, 34, 35F, 35B, 38.

In another preferred embodiment, the medicament is used for the immunization against at least the serotypes 1, 2, 3, 4, 5, 6B, 7F, 8, 9N, 9V, 10A, 11A, 12F, 14, 15B, 17F, 18C, 19A, 19F, 20, 22F, 23F, and 33F, and preferably at least one further serotype, preferably selected from the group of 6A, 7C, 9A, 10B, 13, 15C, 16F, 18B, 21, 23A, 24F, 28F, 31, 34, 35F, 35B, 38.

In a further preferred embodiment, the medicament is used for the immunisation against any of the serotypes given in Table 4, preferably at least 5 or more different serotypes selected from the serotypes given in Table 4, e.g. 8 or more different serotypes, such as 15 or more different serotypes, e.g. 24 or more different serotypes selected from the serotypes given in Table 4.

The immunogenic effect according to the present invention can e.g. be measured by assay of antibodies in serum samples e.g. by a RIA. Furthermore, the effect can be determined in vivo, by measuring e.g. an increased T-cell responsiveness to T-cell dependent antigenic polypeptides, wherein said increased responsiveness is characteristic of an enhancement of a normal immune response to such antigenic polypeptides. An immunostimulating effect may also be measured as an enhanced T cell production of, in particular, IL-2, IL-3, IFN-γ and/or GM-CSF. Polypeptides or fragments thereof having a potential for eliciting an enhanced immune response may thus be readily identified by screening for enhanced IL-2, IL-3, IFN-γ or GM-CSF production by T cells, as described e.g. in U.S. Ser. No. 07/779,499, incorporated herein by reference.

A number of aspects related to vaccination against Streptococcus pneumoniae have been discussed in Bogaert et al. (2004) Vaccine 22:2209-2220. This review includes references to other documents describing methods for testing and evaluation of such vaccines.

The herein described polynucleotides and expression vectors can be introduced into target cells in vivo or in vitro by any standard method: e.g., as naked DNA (Donnelly et al., Annu Rev Immunol 15:617-648 [1997]), incorporated into ISCOMS, liposomes, or erythrocyte ghosts, or by biolistic transfer, calcium precipitation, or electroporation. Alternatively, one can employ a viral-based vector as a means for introducing the polynucleotide encoding the polypeptide of interest Into the cells of the animal or human being. Preferred viral vectors include those derived from replication-defective hepatitis viruses (e.g., HBV and HCV), retroviruses (see, e.g., WO89/07136; and Rosenberg et al., N Eng J Med 323 (9):570-578 [1990]), adenovirus (see, e.g., Morsey et al., J Cell Biochem, Supp. 17E [1993]), adeno-associated virus (Kotin et al., Proc Natl Acad Sci USA 87:2211-2215 [1990]), replication defective herpes simplex viruses (HSV; Lu et al., Abstract, page 66, Abstracts of the Meeting on Gene Therapy, Sep. 22-26, 1992, Cold Spring Harbor Laboratory, Cold Spring Harbor, N.Y.), canary pox virus, and any modified versions of these vectors. Cells transfected in vitro can be cultured and cloned, if desired, prior to introduction into the patient.

In addition to direct in vivo procedures, ex vivo procedures may be used in which cells are removed from an animal, modified, and placed into the same or another animal. It will be evident that one can utilise any of the compositions noted above for introduction of an antigenic polypeptides or polynucleotides encoding such according to the invention into tissue cells in an ex vivo context. Protocols for viral, physical and chemical methods of uptake are well known in the art. Thus, as an alternative to administration of a polypeptide of the invention or a vector capable of expressing such a polypeptide directly to the patient, one can remove helper T cells from the patient; stimulate those T cells ex vivo using the same antigenic polypeptide or vector; and introduce the stimulated helper T cells into the same patient.

### Antibodies and Methods for Raising Antibodies of the Invention

In a further main embodiment, the composition for use as a medicament comprises an antibody capable of binding a polypeptide selected from the group consisting of surface-located Streptococcus pneumoniae polypeptides of SEQ ID NO:1-282, preferably selected from the group consisting of SEQ ID NO:1-41, more preferably selected from the group consisting of SEQ ID NO:16, SEQ ID NO:10, SEQ ID NO:13, SEQ ID NO:20, SEQ ID NO:26, SEQ ID NO:28 and SEQ ID NO:33, most preferably the polypeptide of SEQ ID NO:16. Such a medicament can be used for antibody therapy, such as passive immunisation of an individual in need thereof.

Accordingly, in a further main aspect, the invention relates to antibodies capable of binding, preferably specifically binding, a polypeptide selected from the group consisting of SEQ ID NO:1-282 and/or a fragment and/or a variant thereof ‘Specifically binding’ is, in this context, not intended to mean absolute specificity. The antibody may in some embodiments also specifically bind polypeptides, e.g. from other Streptococcus species, with a high degree of sequence identity to the polypeptide from Streptococcus pneumoniae, e.g. polypeptides with more than 90%, such as more than 95% or more than 98% sequence identity to the polypeptide from Streptococcus pneumoniae.

In a preferred embodiment, the antibody is capable of binding, preferably specifically binding, a polypeptide selected from the group consisting of SEQ ID NO:1-282, such as the polypeptide of SEQ ID NO:1, for example the polypeptide of SEQ ID NO:2, such as the polypeptide of SEQ ID NO:3, for example the polypeptide of SEQ ID NO:4, such as the polypeptide of SEQ ID NO:5, for example the polypeptide of SEQ ID NO:6, such as the polypeptide of SEQ ID NO:7, for example the polypeptide of SEQ ID NO:8, such as the polypeptide of SEQ ID NO:9, for example the polypeptide of SEQ ID NO:10, such as the polypeptide of SEQ ID NO:11, for example the polypeptide of SEQ ID NO:12, such as the polypeptide of SEQ ID NO:13, for example the polypeptide of SEQ ID NO:14, such as the polypeptide of SEQ ID NO:15, for example the polypeptide of SEQ ID NO:16, such as the polypeptide of SEQ ID NO:17, for example the polypeptide of SEQ ID NO:18, such as the polypeptide of SEQ ID NO:19, for example the polypeptide of SEQ ID NO:20, such as the polypeptide of SEQ ID NO:21, for example the polypeptide of SEQ ID NO:22, such as the polypeptide of SEQ ID NO:23, for example the polypeptide of SEQ ID NO:24, such as the polypeptide of SEQ ID NO:25, for example the polypeptide of SEQ ID NO:26, such as the polypeptide of SEQ ID NO:27, for example the polypeptide of SEQ ID NO:28, such as the polypeptide of SEQ ID NO:29, for example the polypeptide of SEQ ID NO:30, such as the polypeptide of SEQ ID NO:31, for example the polypeptide of SEQ ID NO:32, such as the polypeptide of SEQ ID NO:33, for example the polypeptide of SEQ ID NO:34, such as the polypeptide of SEQ ID NO:35, for example the polypeptide of SEQ ID NO:36, such as the polypeptide of SEQ ID NO:37, for example the polypeptide of SEQ ID NO:38, such as the polypeptide of SEQ ID NO:39, for example the polypeptide of SEQ ID NO:40, such as the polypeptide of SEQ ID NO:41, for example the polypeptide of SEQ ID NO:42, such as the polypeptide of SEQ ID NO:43, for example the polypeptide of SEQ ID NO:44, such as the polypeptide of SEQ ID NO:45, for example the polypeptide of SEQ ID NO:46, such as the polypeptide of SEQ ID NO:47, for example the polypeptide of SEQ ID NO:48, such as the polypeptide of SEQ ID NO:49, for example the polypeptide of SEQ ID NO:50, such as the polypeptide of SEQ ID NO:51, for example the polypeptide of SEQ ID NO:52, such as the polypeptide of SEQ ID NO:53, for example the polypeptide of SEQ ID NO:54, such as the polypeptide of SEQ ID NO:55, for example the polypeptide of SEQ ID NO:56, such as the polypeptide of SEQ ID NO:57, for example the polypeptide of SEQ ID NO:58, such as the polypeptide of SEQ ID NO:59, for example the polypeptide of SEQ ID NO:60, such as the polypeptide of SEQ ID NO:61, for example the polypeptide of SEQ ID NO:62, such as the polypeptide of SEQ ID NO:63, for example the polypeptide of SEQ ID NO:64, such as the polypeptide of SEQ ID NO:65, for example the polypeptide of SEQ ID NO:66, such as the polypeptide of SEQ ID NO:67, for example the polypeptide of SEQ ID NO:68, such as the polypeptide of SEQ ID. NO:69, for example the polypeptide of SEQ ID NO:70, such as the polypeptide of SEQ ID NO:71, for example the polypeptide of SEQ ID NO:72, such as the polypeptide of SEQ ID NO:73, for example the polypeptide of SEQ ID NO:74, such as the polypeptide of SEQ ID NO:75, for example the polypeptide of SEQ ID NO:76, such as the polypeptide of SEQ ID NO:77, for example the polypeptide of SEQ ID NO:78, such as the polypeptide of SEQ ID NO:79, for example the polypeptide of SEQ ID NO:80, such as the polypeptide of SEQ ID NO:81, for example the polypeptide of SEQ ID NO:82, such as the polypeptide of SEQ ID NO:83, for example the polypeptide of SEQ ID NO:84, such as the polypeptide of SEQ ID NO:85, for example the polypeptide of SEQ ID NO:86, such as the polypeptide of SEQ ID NO:87, for example the polypeptide of SEQ ID NO:88, such as the polypeptide of SEQ ID NO:89, for example the polypeptide of SEQ ID NO:90, such as the polypeptide of SEQ ID NO:91, for example the polypeptide of SEQ ID NO:92, such as the polypeptide of SEQ ID NO:93, for example the polypeptide of SEQ ID NO:94, such as the polypeptide of SEQ ID NO:95, for example the polypeptide of SEQ ID NO:96, such as the polypeptide of SEQ ID NO:97, for example the polypeptide of SEQ ID NO:98, such as the polypeptide of SEQ ID NO:99, for example the polypeptide of SEQ ID NO:100, such as the polypeptide of SEQ ID NO:101, for example the polypeptide of SEQ ID NO:102, such as the polypeptide of SEQ ID NO:103, for example the polypeptide of SEQ ID NO:104, such as the polypeptide of SEQ ID NO:105, for example the polypeptide of SEQ ID NO:168.

In preferred embodiments, the antibodies of the invention are furthermore capable of binding an intact Streptococcus pneumoniae cell, i.e. capable of binding a living or a dead Streptococcus cell which has maintained its structural integrity, preferably a cell that has maintained the integrity of the membrane (i.e. wherein the membrane has not been permeabilized). Binding of antibodies to intact cells can e.g. be determined by flow cytometry as described in Rioux et al. (2001) Infect. Immun. 69:5162-5165 or as described in Singh et al. (2003) Infect. Immun. 71:3937-3946.

Preferred antibodies are ones that bind with a dissociation constant or Kd of less than 5×10⁻⁸M, such as less than 10⁻⁶M, e.g. less than 5×10⁻⁷M, such as less than 10⁻⁷M, e.g. less than 5×10⁻⁸M, such as less than 10⁻⁸M, e.g. less than 5×10⁻⁹M, such as less than 10⁻⁹M, e.g. less than 5×10⁻¹⁰M, such as less than 10⁻¹⁰M, e.g. less than 5×10⁻¹¹M, such as less than 10⁻¹¹M, e.g. less than 5×10⁻¹²M, such as less than 10⁻¹²M, e.g. less than 5×10⁻¹³M, such as less than 10⁻¹³M, e.g. less than 5×10⁻¹⁴M, such as less than 10⁻¹⁴M, e.g. less than 5×10⁻¹⁵M, or less than 10⁻¹⁵M. Binding constants can be determined using methods well-known in the art, such as ELISA (e.g. as described in Orosz and Ovadi (2002) J. Immunol. Methods 270:155-162) or surface plasmon resonance analysis.

Antibodies can be used for passive immunisation of mammals, preferably human beings, more preferably immunocompromised patients. A treatment with antibodies can be done to cure or to prevent Streptococcus pneumoniae infections, including pneumococcal diseases, such as pneumonia or meningitis or pneumococcal sepsis. Preferred patient groups include children under the age of 4 years, elderly patients or immunocompromised patients.

Antibodies of the invention include the following preferred mechanistic groups:
1. Function-inhibiting antibodies that work as an antibacterial (affect the viability of the bacterium). Such antibodies should be effective regardless of the immune status of the patient. Preferably, such antibodies are capable of reducing Streptococcus pneumoniae growth in vitro to less than 50%, such as less than 25%, for example less than 10%, such as less than 5% of a control without antibody added.
2. Opsonising antibodies that are designed to enhance phagocytic killing. Effectiveness of such antibodies may depend on the immune status of the patient, but it is very well possible that they will enhance phagocytic killing even in compromised patients.
3. Antibodies conjugated to a therapeutic moiety such as a toxin or bactericidal agent, e.g. ricin or radioisotopes. Techniques for conjugating a therapeutic moiety to antibodies are well known, see, e.g. Thorpe et al. (1982) Immunol. Rev. 62, 119-158. These antibodies should also be effective regardless of the immune status of the patient.
   An antibody with or without a therapeutic moiety conjugated to it can be used as a therapeutic that is administered alone or in combination with chemotherapeutics or other therapeutic agents.

In one embodiment, the antibodies of the invention are opsonising as well as function-inhibiting. In another embodiment, the antibodies of the invention are opsonising, but not function-inhibiting. The latter group of antibodies can e.g. be antibodies directed against a target polypeptide which is not essential for the viability of Streptococcus pneumoniae.

In a further main aspect, the invention relates to a method for raising antibodies to a polypeptide selected from the group consisting of SEQ ID NO:1-282, in a non-human animal comprising the steps of
a. providing
   a polypeptide comprising a sequence selected from the group consisting of SEQ ID NO:1-282, preferably selected from the group consisting of SEQ ID NO:16, SEQ ID NO:10, SEQ ID NO:13, SEQ ID NO:20, SEQ ID NO:26, SEQ ID NO:28 and SEQ ID NO:33, most preferably SEQ ID NO:16, or comprising an antigenic fragment or variant of said sequence,
   a polynucleotide comprising a sequence encoding said polypeptide,
   an expression vector comprising a sequence encoding said polypeptide,
   or
   a recombinant virus or recombinant cell comprising said polynucleotide or said expression vector,
b. introducing a composition comprising said polypeptide, polynucleotide, vector, recombinant virus or recombinant cell into said animal,
c. raising antibodies in said animal,
d. isolating and optionally purifying the antibodies.

In a preferred embodiment, antibodies capable of binding an intact Streptococcus pneumoniae cell are identified by comprising performing the above steps and the further step of selecting antibodies capable of binding an intact Streptococcus pneumoniae cell.

The above methods are preferably done in a transgenic animal which is capable of producing human antibodies. In a further preferred embodiment, the above methods are non-therapeutic.

### Monoclonal/Polyclonal Antibodies

Antibodies of the invention may be polyclonal antibodies or monoclonal antibodies or mixtures of monoclonal antibodies. In a preferred embodiment, the antibody is a monoclonal antibody or a fragment thereof. Monoclonal antibodies (Mab's) are anti-bodies wherein every antibody molecule is similar and thus recognises the same epitope. The antibody may be any kind of antibody, however, it is preferably an IgG or IgA antibody.

Preferred antibodies, more preferably monoclonal antibodies, are antibodies capable of specifically binding surface-exposed regions of the polypeptides of the invention. Accordingly, in a preferred embodiment of an antibody capable of binding SEQ ID NO:16, said antibody binds an epitope on SEQ ID NO:16 which comprises one or more amino acids of any of SEQ ID NO:289-SEQ ID NO:294. Even more preferably, said antibody binds an epitope which comprises two or more, such as three or more, e.g., four or more, such as five or more amino acids of a sequence selected from the group consisting of SEQ ID NO:289, SEQ ID NO:290, SEQ ID NO:291, SEQ ID NO:292, SEQ ID NO:293 and SEQ ID NO:294.

Monoclonal antibodies are in general produced by a hybridoma cell line. Methods of making monoclonal antibodies and antibody-synthesising hybridoma cells are well known to those skilled in the art. Antibody-producing hybridomas may for example be prepared by fusion of an antibody-producing B lymphocyte with an immortalised cell line. A monoclonal antibody can be produced by the following steps. An animal is immunised with an antigen such as a full-length polypeptide or a fragment thereof. The immunisation is typically accomplished by administering the antigen to an immunologically competent mammal in an immunologically effective amount, i.e., an amount sufficient to produce an immune response. Preferably, the mammal is a rodent such as a rabbit, rat or mouse. The mammal is then maintained on a booster schedule for a time period sufficient for the mammal to generate high affinity antibody molecules. A suspension of antibody-producing cells is removed from each immunised mammal secreting the desired antibody. After a sufficient time to generate high affinity antibodies, the animal (e.g. mouse) is sacrificed and antibody-producing lymphocytes are obtained from one or more of the lymph nodes, spleens and peripheral blood. Spleen cells are preferred, and can be mechanically separated into individual cells in a physiological medium using methods well known to one of skill in the art. The antibody-producing cells are immortalised by fusion to cells of a mouse myeloma line. Mouse lymphocytes give a high percentage of stable fusions with mouse homologous myelomas, however, rat, rabbit and frog somatic cells can also be used. Spleen cells of the desired antibody-producing animals are immortalised by fusing with myeloma cells, generally in the presence of a fusing agent such as polyethylene glycol. Any of a number of myeloma cell lines suitable as a fusion partner can be, for example, the P3-NS1/1-Ag4-1, P3-x63-Ag8.653 or Sp2/O—Ag14 myeloma lines, available from the American Type Culture Collection (ATCC), Rockville, Md.

Monoclonal antibodies can also be generated by other methods well known to those skilled in the art of recombinant DNA technology. An alternative method, referred to as the “combinatorial antibody display” method, has been developed to identify and isolate antibody fragments having a particular specificity, and can be utilised to produce monoclonal antibodies.

A polyclonal antibody is a mixture of antibody molecules recognising a specific given antigen, hence polyclonal antibodies may recognise different epitopes within e.g. a polypeptide. In general polyclonal antibodies are purified from serum of a mammal, which previously has been immunised with the antigen. Polyclonal antibodies may for example be prepared by any of the methods described in Antibodies: A Laboratory Manual, By Ed Harlow and David Lane, Cold Spring Harbor Laboratory Press, 1988. Polyclonal antibodies may be derived from any suitable mammalian species, for example from mice, rats, rabbits, donkeys, goats, and sheep.

### Specificity

The antibodies of the invention may be monospecific towards any of the polypeptides of SEQ ID NO:1-282. In another embodiment, the antibody is bispecific or multispecific having at least one portion being specific towards any of the polypeptides of SEQ ID NO:1-282.

Monospecific antibodies may be monovalent, i.e. having only one binding domain. For a monovalent antibody, the immunoglobulin constant domain amino-acid sequences preferably comprise the structural portions of an antibody molecule known in the art as CH1, CH2, CH3 and CH4. Preferred are those which are known in the art as C_{L}. Furthermore, insofar as the constant domain can be either a heavy or light chain constant domain (C_{H} or C_{L}, respectively), a variety of monovalent antibody compositions are contemplated by the present invention. For example, light chain constant domains are capable of disulphide bridging to either another light chain constant domain, or to a heavy chain constant domain. In contrast, a heavy chain constant domain can form two independent disulphide bridges, allowing for the possibility of bridging to both another heavy chain and to a light chain, or to form polymers of heavy chains. Thus, in another embodiment, the invention contemplates a composition comprising a monovalent polypeptide wherein the constant chain domain C has a cysteine residue capable of forming at least one disulphide bridge, and where the composition comprises at least two monovalent polypeptides covalently linked by said disulphide bridge.

In another embodiment of the invention the antibody is a multivalent antibody having at least two binding domains. The binding domains may have specificity for the same ligand or for different ligands.

### Multispecificity, Including Bispecificity

In a preferred embodiment the invention relates to multispecific antibodies, which have affinity for and are capable of specifically binding at least two different entities.

In one embodiment, the multispecific antibody is a bispecific antibody, which carries at least two different binding domains, at least one of which is of antibody origin. A bispecific molecule of the invention can also be a single chain bispecific molecule. Multispecific molecules can also be single-chain molecules or may comprise at least two single-chain molecules. The multispecific, including bispecific antibodies, may be produced by any suitable manner known to the person skilled in the art. A number of approaches have been developed such as the ones described in WO 94/09131; WO 94/13804; WO 94/13806 or U.S. Pat. Nos. 5,260,203; 5,455,030; 4,881,175; 5,132,405; 5,091,513; 5,476,786; 5,013,653; 5,258,498; and 5,482,858. Using a bispecific or multispecific antibody according to the invention the invention offers several advantages as compared to monospecific/monovalent antibodies. A bispecific/multispecific antibody has a first binding domain capable of specifically recognising and binding any of the Streptococcus pneumoniae polypeptides of SEQ ID NO:1-282, whereas the other binding domain(s) may be used for other purposes. In one embodiment, at least one other binding domain is used for binding to a Streptococcus pneumoniae polypeptide, such as binding to another epitope on the same Streptococcus pneumoniae polypeptide as the first binding domain. Thereby specificity for Streptococcus pneumoniae may be increased as well as increase of avidity of the antibody. In another embodiment the at least one other binding domain may be used for specifically binding a mammalian cell, such as a human cell. It is preferred that the at least other binding domain is capable of binding an immunoactive cell, such as a leukocyte, a macrophage, a lymphocyte, a basophilic cell, and/or an eosinophilic cell, in order to increase the effect of the antibody in a therapeutic method. This may be accomplished by establishing that the at least one other binding domain is capable of specifically binding a mammalian protein, such as a human protein, such as a protein selected from any of the cluster differentiation proteins (CD), in particular CD64 and/or CD89.

### Humanised Antibodies

It is not always desirable to use non-human antibodies for human therapy, since the non-human “foreign” epitopes may elicit an immune response in the individual to be treated. To eliminate or minimise the problems associated with non-human antibodies, it is desirable to engineer chimeric antibody derivatives, i.e., “humanised” antibody molecules that combine the non-human Fab variable region binding determinants with a human constant region (Fc). Such antibodies are characterised by equivalent antigen specificity and affinity of the monoclonal and polyclonal antibodies described above, and are less immunogenic when administered to humans, and therefore more likely to be tolerated by the individual to be treated.

Accordingly, in one embodiment the antibody of the invention is a humanised antibody. Humanised antibodies are in general chimeric antibodies comprising regions derived from a human antibody and regions derived from a non-human antibody, such as a rodent antibody. Humanisation (also called Reshaping or CDR-grafting) is a well-established technique for reducing the immunogenicity of monoclonal antibodies (mAbs) from xenogeneic sources (commonly rodent), increasing the homology to a human immunoglobulin, and for improving their activation of the human immune system. Thus, humanised antibodies are typically human antibodies in which some CDR residues and possibly some framework residues are substituted by residues from analogous sites in rodent antibodies.

It is important that humanised antibodies retain high affinity for the antigen and other favourable biological properties. To achieve this goal, according to a preferred method, humanised antibodies are prepared by a process of analysis of the parental sequences and various conceptual humanised products using three-dimensional models of the parental and humanised sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of certain residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is maximised, although it is the CDR residues that directly and most substantially influence antigen binding.

One method for humanising MAbs relates to production of chimeric antibodies in which an antigen binding site comprising the complete variable domains of one antibody is fused to constant domains derived from a second antibody, preferably a human antibody. Methods for carrying out such chimerisation procedures are for example described in EP-A-0 120 694 (Celltech Limited), EP-A-0 125 023 (Genentech Inc.), EP-A-0 171 496 (Res. Dev. Corp. Japan), EP-A-0173494 (Stanford University) and EP-A-0 194 276 (Celltech Limited).

The humanised antibody of the present invention may be made by any method capable of replacing at least a portion of a CDR of a human antibody with a CDR derived from a non-human antibody. Winter describes a method which may be used to prepare the humanised antibodies of the present invention (UK Patent Application GB 2188638A), the contents of which are incorporated by reference.

As an example, the humanised antibodies of the present invention may be produced by the following process:
(a) constructing, by conventional techniques, an expression vector containing an operon with a DNA sequence encoding an antibody heavy chain in which the CDRs and such minimal portions of the variable domain framework region that are required to retain antibody binding specificity are derived from a non-human immunoglobulin, and the remaining parts of the antibody chain are derived from a human immunoglobulin;
(b) constructing, by conventional techniques, an expression vector containing an operon with a DNA sequence encoding a complementary antibody light chain in which the CDRs and such minimal portions of the variable domain framework region that are required to retain donor antibody binding specificity are derived from a non-human immunoglobulin, and the remaining parts of the antibody chain are derived from a human immunoglobulin;
(c) transfecting the expression vectors into a host cell by conventional techniques; and
(d) culturing the transfected cell by conventional techniques to produce the humanised antibody.

The host cell may be co-transfected with the two vectors of the invention, the first vector containing an operon encoding a light chain derived polypeptide and the second vector containing an operon encoding a heavy chain derived polypeptide. The two vectors contain different selectable markers, but otherwise, apart from the antibody heavy and light chain coding sequences, are preferably identical, to ensure, as far as possible, equal expression of the heavy and light chain polypeptides. Alternatively, a single vector may be used, the vector including the sequences encoding both the light and the heavy chain polypeptides. The coding sequences for the light and heavy chains may comprise cDNA or genomic DNA or both.

The host cell used to express the altered antibody of the invention may be either a bacterial cell such as Escherichia coli, or a eukaryotic cell. In particular a mammalian cell of a well defined type for this purpose, such as a myeloma cell or a Chinese hamster ovary cell may be used.

The general methods by which the vectors of the invention may be constructed, transfection methods required to produce the host cell of the invention and culture methods required to produce the antibody of the invention from such host cells are all conventional techniques. Likewise, once produced, the humanised antibodies of the invention may be purified according to standard procedures.

### Human Antibodies

In a more preferred embodiment the invention relates to an antibody, Wherein the binding domain is carried by a human antibody, i.e. wherein the antibodies have a greater degree of human peptide sequences than do humanised antibodies.

Human mAb antibodies directed against human proteins can be generated using transgenic mice carrying the human immune system rather than the mouse system. Splenocytes from these transgenic mice immunised with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326). Such transgenic mice are available from Abgenix, Inc., Fremont, Calif., and Medarex, Inc., Annandale, N.J. It has been described that the homozygous deletion of the antibody heavy-chain joining region (1H) gene in chimeric and germ-line mutant mice results in complete inhibition of endogenous antibody production. Transfer of the human germ-line immunoglobulin gene array in such germ-line mutant mice will result in the production of human anti-bodies upon antigen challenge. See, e.g., Jakobovits et al., Proc. Natl. Acad. Sci. USA 90:2551 (1993); Jakobovits et al., Nature 362:255-258 (1993); Bruggemann et al., Year in Immunol. 7:33 (1993); and Duchosal et al. Nature 355:258 (1992). Human antibodies can also be derived from phage-display libraries (Hoogenboom et al., J. Mol. Biol. 227: 381 (1992); Marks et al., J. Mol. Biol. 222:581-597 (1991); Vaughan, et al., Nature Biotech 14:309 (1996)).

A preferred method for the isolation of high affinity antibodies is a subtractive procedure were human antibodies or antibody fragments against the targets, in particular against the antigens 029 (SEQ ID NO:16) and 607 (SEQ ID NO:20), in their native configuration can be rapidly obtained from a phage antibody library (see, e.g. De Kruif et al., Proc. Natl. Acad. Sci. USA 92:3938-3942 (1995); U.S. Pat. No. 6,265,150; and US patent applications 2002132228 and 2005043521). The phage antibody libraries can e.g. be constructed using antibody producing cells from patients with the disease of interest, here patients infected with Streptococcus pneumoniae. The genes coding for the antibodies produced by these cells may be cloned into a semi-synthetic phage antibody library using degenerated oligonucleotides rearranging the CDR3 Region of the cloned genes. Afterwards the library is incubated with the target antigen or target-expressing cells, here Streptococcus pneumoniae, and the phage antibodies bound to the target are isolated by using standard methods. The present invention is also directed to antibodies identified by the above procedure, in particular antibodies capable of binding a polypeptide selected from group consisting of SEQ ID NO:16, SEQ ID NO:10, SEQ ID NO:13, SEQ ID NO:20, SEQ ID NO:26, SEQ ID NO:28 and SEQ ID NO:33, preferably antibodies having a dissociation constant or Kd of less than 10⁻⁷M, e.g. less than 10⁻⁸M, such as less than 10⁻⁹M, e.g. less than 10⁻¹⁰M, such as less than 10⁻¹¹M and/or antibodies binding a surface-exposed epitope of any of these targets.

Suitable methods for producing human monoclonal antibodies have furthermore been described in WO 03/017935, WO 02/100348, US 2003 091561, and US 2003 194403.

### Binding Fragments of Antibodies

In one embodiment of the invention, the antibody is a fragment of an antibody, preferably an antigen binding fragment or a variable region. Examples of antibody fragments useful with the present invention include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallise readily. Pepsin treatment yields an F(ab′)₂ fragment that has two antigen binding fragments which are capable of cross-linking antigen, and a residual other fragment (which is termed pFc′). Additional fragments can include diabodies, linear antibodies, single-chain antibody molecules, and multispecific antibodies formed from antibody fragments.

The antibody fragments Fab, Fv and scFv differ from whole antibodies in that the antibody fragments carry only a single antigen-binding site. Recombinant fragments with two binding sites have been made in several ways, for example, by chemical cross-linking of cysteine residues introduced at the C-terminus of the VH of an Fv (Cumber et al., 1992), or at the C-terminus of the VL of an scFv (Pack and Pluckthun, 1992), or through the hinge cysteine residues of Fab's (Carter et al., 1992).

Preferred antibody fragments retain some or essentially all of the ability of an antibody to selectively binding with its antigen. Some preferred fragments are defined as follows:
(1) Fab is the fragment that contains a monovalent antigen-binding fragment of an antibody molecule. A Fab fragment can be produced by digestion of whole anti-body with the enzyme papain to yield an intact light chain and a portion of one heavy chain.
(2) Fab′ is the fragment of an antibody molecule and can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.
(3) (Fab′)₂ is the fragment of an antibody that can be obtained by treating whole anti-body with the enzyme pepsin without subsequent reduction. F(ab′)₂ is a dimer of two Fab′ fragments held together by two disulfide bonds.
(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognise and bind antigen, although at a lower affinity than the entire binding site.

In one embodiment of the present invention the antibody is a single-chain antibody, defined as a genetically engineered molecule containing the variable region of the light chain, the variable region of the heavy chain, linked by a suitable polypeptide linker as a genetically fused single chain molecule. Such single-chain antibodies are also referred to as “single-chain Fv” or “sFv” antibody fragments. Generally, the Fv polypeptide further comprises a polypeptide linker between the V_{H} and V_{L} domains that enables the sFv to form the desired structure for antigen binding.

The antibody fragments according to the invention may be produced in any suitable manner known to the person skilled in the art. Several microbial expression systems have already been developed for producing active antibody fragments, e.g. the production of Fab in various hosts, such as E. coli or yeast has been described, The fragments can be produced as Fab's or as Fv's, but additionally it has been shown that a V_{H} and a V_{L} can be genetically linked in either order by a flexible polypeptide linker, which combination is known as an scFv.

### Compositions for Use in the Invention

In a preferred embodiment of the composition for use as a medicament, said composition comprises, in addition to the active component, a pharmaceutically-acceptable carrier.

As used herein, the term “pharmaceutically acceptable” used in connection with compositions or carriers represents that the materials are capable of being administered to or upon a human or animal without the production of undesirable physiological effects such as nausea, dizziness, gastric upset and the like.

The preparation of a composition that contains active ingredients dissolved or dispersed therein is well understood in the art. Often such compositions are prepared as sterile injectables either as liquid solutions or suspensions, aqueous or non-aqueous, however, solid forms suitable for solution, or suspension, in liquid prior to use can also be prepared. The preparation can also be emulsified. The active ingredient can be mixed with carriers which are pharmaceutically acceptable and compatible with the active ingredient and in amounts suitable for use in the methods described herein. Suitable carriers are, for example, water, saline, dextrose, glycerol, ethanol or the like and combinations thereof. In addition, if desired, the composition can contain minor amounts of auxiliary substances such as wetting or emulsifying agents, pH-buffering agents and the like which enhance the effectiveness of the active ingredient.

The compositions of the present invention can include pharmaceutically-acceptable salts of the active components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Pharmaceutically-acceptable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes. Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

The composition may also be a kit-in-part further including an antibiotic agent, such as antibiotics selected from vancomycin, β-lactams, cephalosporins, penicillins, aminoglycosides, macrolide antibiotics (erythromycin, clarithromycin, or azithromycin) and fluoroquinolone antibiotics (ciprofloxacin, levofloxacin, gatifloxacin, or moxifloxacin) and/or including an immunostimulating agent, such as cytokines, interferons, growth factors, for example GCSF or GM-CSF. The kit-in-part may be used for simultaneous, sequential or separate administration.

The Invention furthermore relates to pharmaceutical compositions useful for practising the methods described herein. Thus, the invention relates to a pharmaceutical composition comprising a pharmaceutically-acceptable carrier and
an isolated polypeptide which comprises any of the sequences of SEQ ID NO:1-282, preferably selected from the group consisting of SEQ ID NO:16, SEQ ID NO:10, SEQ ID NO:13, SEQ ID NO:20, and SEQ ID NO:28, most preferably SEQ ID NO:16, or comprises a fragment or variant of any of said sequences,
an isolated polynucleotide comprising a sequence encoding said polypeptide,
an expression vector comprising a sequence encoding said polypeptide,
or
a recombinant virus or recombinant cell comprising said polynucleotide or said expression vector.

Furthermore, the invention relates to a pharmaceutical composition comprising an antibody of the invention, preferably an antibody capable of binding a polypeptide selected from the group consisting of SEQ ID NO:16, SEQ ID NO:10, SEQ ID NO:13, SEQ ID NO:20, SEQ ID NO:26, SEQ ID NO:28 and SEQ ID NO:33, most preferably SEQ ID NO:16, as defined herein and a pharmaceutically-acceptable carrier.

### Polypeptides of the Invention

### Fragments of the Invention

In a further aspect, the invention relates to a fragment, preferably an antigenic fragment, of a polypeptide set forth in any of SEQ ID NO:1-282, preferably SEQ ID NO:16. Antigenicity can be predicted by various methods known in the art. The length of such fragments may vary from 2 consecutive amino-acid residues of a polypeptide to the full-length polypeptide minus one amino-acid residue. Preferably, fragments are less than 100 consecutive amino acids, such as less than 70 or 50 consecutive amino acids, e.g. less than consecutive 40 amino acids, such as less than 30 consecutive amino acids, e.g. less than 25 consecutive amino acids, such as less than consecutive 20 amino acids in length. Thus, for example fragments can be 2, 3, 4, 5, 6, 7, 8, 9, 10, 11, 12, 13, 14, 15, 16, 17, 18, 19 or 20 consecutive amino acids in length. In further preferred embodiments, a fragment comprises 6 or more, such as 7 or more, e.g. 8 or more, such as 9 or more, e.g. 10 or more consecutive amino acids of the corresponding full-length sequence. Preferred ranges include fragments of between 5 and 50 consecutive amino acids in length, such as between 5 and 25 consecutive amino acids in length, e.g. between 5 and 20 consecutive amino acids in length. Expressed in another way, a fragment consists of a part of an amino-acid sequence which is less than 100% in length as compared to the full-length polypeptide. Preferably, the length of the fragment is less than 99%, such as less than 75%, e.g. less than 50%, such as less than 25%, e.g. less than 20%, such as less than 15%, e.g. less than 10% of the length of the full-length polypeptide. In further preferred embodiments, the fragment consists of a part of an amino-acid sequence which is less than 100%, but more than 1% in length as compared to the full-length polypeptide, such as less than 100% but more than 5%, e.g. less than 100% but more than 10%, such as less than 100% but more than 20%, e.g. less than 100% but more than 25%, such as less than 100% but more than 50% of the length of the full-length polypeptide.

Preferably, fragments of the invention are surface-exposed in an intact Streptococcus pneumoniae cell or other cell when expressed recombinantly therein. Surface-exposure can be e.g. be determined using a monoclonal antibody specific for said fragment, e.g. as described in Singh et al. (2003) Infect. Immun. 71:3973-3946. Also preferred are fragments which are capable of inducing antibodies that can specifically bind an intact Streptococcus pneumoniae cell. This can be determined by generating monoclonal antibodies using said fragment and subsequent characterisation of the binding of individual antibodies to Intact cells, e.g. as described in Singh et al. (2003) Infect. Immun. 71:3973-3946. Preferred fragments of SEQ ID NO:16 include fragments comprising or consisting of one or more of the sequences of SEQ ID NO:289-SEQ ID NO:294, more preferred fragments include fragments comprising or consisting of SEQ ID NO:289 and/or SEQ ID NO:290, fragments comprising or consisting of SEQ ID NO: 291 and/or SEQ ID NO: 292, and fragments comprising or consisting of SEQ ID NO:293 and/or SEQ ID NO:294.

The full-length polypeptides of SEQ ID NO:1-282 as well as the fragments of the invention can be produced recombinantly by conventional techniques known in the art. Suitable host cells can be mammalian cells, e.g. CHO, COS or HEK293 cells. Alternatively, insect cells, bacterial cells or fungal cells can be used. Methods for heterologous expression of polynucleotide sequences in the cell types listed above and subsequent purification of the produced polypeptides, e.g. using a tag sequence such as a histidine tag, which may be removed after purification, are well-known to those skilled in the art. Alternatively, fragments of the invention can be produced synthetically.

### Variants of the Invention

In a further main aspect, the invention relates to the use of variants of any of the polypeptides set forth in SEQ ID NO:1-282, preferably SEQ ID NO:16, or variants of fragments of any of the polypeptides set forth in SEQ ID NO:1-282, preferably SEQ ID NO:16, in a composition for use as a medicament. When used herein, phrases such as ‘a polypeptide having at least 95% sequence identity to SEQ ID NO:X’ are used interchangeably with, and are intended to be directed to the same subject-matter as, phrases such as ‘the polypeptide of SEQ ID NO:X and variants thereof, wherein the variant has at least 95% sequence identity to said sequence.’

Variants preferably have at least 75% sequence identity, for example at least 80% sequence identity, such as at least 85% sequence identity, for example at least 90% sequence identity, such as at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence Identity, for example 99% sequence identity with the given polypeptide or fragment. Sequence identity is determined with any of the algorithms GAP, BESTFIT, or FASTA in the Wisconsin Genetics Software Package Release 7.0, using default gap weights.

Preferred variants of a given polypeptide or fragment are variants in which all amino-acid substitutions between the variant and the given reference polypeptide or fragment are conservative substitutions. Conservative amino-acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine, a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino-acids substitution groups are: valine-leucine-isoleucine, phenylalanine-tyrosine, lysine-arginine, alanine-valine, and asparagine-glutamine.

Variants of a polypeptide or of a fragment thereof also include forms of the polypeptide or fragment wherein one or more amino acids have been deleted or inserted. Preferably, less than 5, such as less than 4, e.g. less than 3, such as less than 2, e.g. only one amino acid has been inserted or deleted. ‘Variants’ of a polypeptide or of a fragment thereof also include forms of these polypeptides or fragments modified by post-translational modifications of the amino-acid sequence.

### Recombinant Cells of the Invention

In a further main aspect, the invention relates to the use of a recombinant cell transformed or transfected with a polynucleotide comprising a sequence encoding a polypeptide, said polypeptide comprising a sequence selected from the group consisting of SEQ ID NO:1-282, preferably SEQ ID NO:16, or comprising an antigenic fragment or variant of said sequence. Preferably, said recombinant cell is an Escherichia coli or Salmonella cell, more preferably an attenuated or reduced-virulence Escherichia or Salmonella cell.

Suitable bacterial strains for use herein have been described in e.g. Makino et al. (2001) Microb. Pathog. 31:1-8; Gentschev et al. (2002) Int. J. Med. Microbiol. 291:577-582; Turner et al. (2001) Infect. Immun. 69:4969-4979; WO99/49026; and WO03/022307 and references therein. Examples of suitable Salmonella strains are CvD908-T7pol (Santiago-Machuca et al. (2002) Plasmid 47:108-119), ATCC 39183, ATCC 53647 and ATCC 53648. Examples of suitable E. coli strains are YT106 and E1392/75-2A.

### METHODS AND USES OF THE INVENTION

The compositions and other products defined above can be used to treat or prevent Streptococcus pneumoniae infections, and/or disease resulting from such infections, In animals or human beings in need thereof.

Treatment and prevention herein include all types of therapeutic treatment and preventive treatment and other treatments to combat Streptococcus pneumoniae, including but not limited to vaccination, prophylaxis, active immunisation, passive immunisation, administration of antibodies, curative treatment, ameliorating treatment. In particular, passive immunisation using antibodies of the invention is a suitable treatment for immunocompromised individuals.

### Diagnostic Methods of the Invention

The combination of being surface-exposed and being present in relatively high copy numbers in cells also makes the polypeptides identified by the inventors highly suitable as targets for detection of Streptococcus pneumoniae, allowing detection of this organism with high sensitivity.

Accordingly, in a further main aspect, the invention relates to a method for detecting Streptococcus pneumoniae or parts thereof in a sample comprising the steps of
a. contacting said sample with an indicator moiety capable of specifically binding a polypeptide selected from the group consisting of SEQ ID NO:1-282, preferably selected from the group consisting of SEQ ID NO:16, SEQ ID NO:10, SEQ ID NO:13, SEQ ID NO:20, SEQ ID NO:26, SEQ ID NO:28 and SEQ ID NO:33, most preferably SEQ ID NO:16, and
b. determining whether a signal has been generated by the indicator moiety, thereby detecting whether said sample contains Streptococcus pneumoniae or parts thereof.
   Preferably, said indicator moiety is capable of binding, preferably specifically binding, intact Streptococcus pneumoniae cells.

In preferred embodiments of the above diagnostic methods, a washing step is performed between the contacting step and the determination step, in order to improve the specificity of detection.

The sample can e.g. be feces, urine, a tissue, tissue extract, fluid sample or body fluid sample, such as blood, plasma, serum, sputum, or a sample taken from nose or lung. Another example of a sample is a food sample, such as a meat sample.

In another aspect, the invention relates to a method for detecting Streptococcus pneumoniae or parts thereof in a sample comprising the step of analysing a sample by mass spectrometry to evaluate the presence and/or quantity of one or more of the polypeptides of SEQ ID NO:1-282, in particular SEQ ID NO:16 and/or SEQ ID NO:20. In one embodiment, the sample, e.g. a blood sample, is pre-treated to enrich for the polypeptide(s) to be detected. Such a pre-treatment may include a size-fractionation of proteins present in the sample.

The above methods can e.g. be used to diagnose Streptococcus pneumoniae infections in an individual. In preferred embodiments of the above methods, said indicator moiety does not pass through the membrane of a Streptococcus pneumoniae cell. A preferred type of said indicator moiety consists of or comprises an antibody, such as an antibody of the invention as defined herein.

Those skilled in the art will understand that there are numerous well known clinical diagnostic chemistry procedures in which an indicator moiety can be used to form an binding reaction product whose amount relates to the amount of the ligand, herein Streptococcus pneumoniae or parts thereof, in a sample. Thus, while exemplary assay methods are described herein, the invention is not so limited.

The present invention also relates to a diagnostic system, preferably in kit form, for assaying for the presence, and preferably also the amount, of Streptococcus pneumoniae in a biological sample. Methods for the preparation of diagnostic kits have e.g. been described in U.S. Pat. No. 5,470,958 and references therein.

The diagnostic system includes, in an amount sufficient to perform at least one assay, an indicator moiety according to the present invention, preferably as a separately packaged reagent, and more preferably also instructions for use. Packaged refers to the use of a solid matrix or material such as glass, plastic (e.g., polyethylene, polypropylene or polycarbonate), paper, foil and the like capable of holding within fixed limits an indicator moiety of the present invention. Thus, for example, a package can be a glass vial used to contain milligram quantities of a contemplated labelled indicator moiety preparation, or it can be a microtiter plate well to which microgram quantities of a contemplated indicator moiety has been operatively affixed, i.e., linked so as to be capable of binding a ligand.

“Instructions for use” typically include a tangible expression describing the reagent concentration or at least one assay method parameter such as the relative amounts of reagent and sample to be admixed, maintenance time periods for reagent-sample admixtures, temperature, buffer conditions and the like.

In most embodiments, the diagnostic method and system of the present invention include as a part of the Indicator moiety, a label or indicating means capable of signalling the formation of a binding reaction complex containing an indicator moiety complexed with the preselected ligand (i.e. a polypeptide comprising any of the sequences of SEQ ID NO:1-282 and/or a fragment thereof). Such labels are themselves well-known in clinical diagnostic chemistry.

The labelling means can be a fluorescent labelling agent that chemically binds to antibodies or antigens without denaturing them to form a fluorochrome (dye) that is a useful immunofluorescent tracer. Suitable fluorescent labelling agents are fluorochromes such as fluorescein isocyanate (FIC), fluorescein isothiocyante (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC). Other examples of suitable fluorescent materials include umbelliferone, dichlorotriazinylamine fluorescein, dansyl chloride or phycoerythrin and the like. A description of immunofluorescence analysis techniques is found in DeLuca, “Immunofluorescence Analysis”, in Antibody As a Tool, Marchalonis, et al., eds., John Wiley & Sons, Ltd., pp. 189-231 (1982).

Radioactive elements can be useful as labelling agents. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Particularly preferred is ¹²⁵I. Another group of useful labelling means are those elements such as ¹¹C, ¹⁸F, ¹⁵O and ¹³N which themselves emit positrons, or beta emitters, such as ¹¹¹indium of ³H. Other suitable radioactive materials include ¹³¹I and ³⁵S.

Detection using antibodies can, in other embodiments, be facilitated by coupling the antibody to another detectable substance, such as an enzyme, a prosthetic group, a luminescent materials, or a bioluminescent material. Examples of suitable enzymes include horseradish peroxidase, alkaline phosphatase, beta-galactosidase, or acetylcholinesterase; examples of suitable prosthetic group complexes include Streptavidin/biotin and avidin/biotin; an example of a luminescent material includes luminol; examples of bioluminescent materials include luciferase, luciferin, and aequorin.

In preferred embodiments, the indicating group is an enzyme, such as horseradish peroxidase (HRP) or glucose oxidase. In such cases where the principal indicating group is an enzyme such as HRP or glucose oxidase, additional reagents are required to visualise the fact that a indicator-moiety/ligand complex (immunoreactant) has formed. Such additional reagents for HRP include hydrogen peroxide and an oxidation dye precursor such as diaminobenzidine. An additional reagent useful with glucose oxidase is 2,2′-amino-di-(3-ethyl-benzthiazoline-G-sulfonic acid).

The linking of labels, i.e. labelling of polypeptides such as antibodies, is well known in the art. For instance, proteins can be labelled by metabolic incorporation of radioisotope-containing amino acids provided as a component in the culture medium. See, for example, Galfre et al., Meth. Enzymol., 73:3-46 (1981). The techniques of protein conjugation or coupling through activated functional groups are particularly applicable. See, for example, Aurameas, et al., Scand. J. Immunol., Vol. 8 Suppl. 7:7-23 (1978), Rodwell et al. (1984) Biotech. 3:889-894, and U.S. Pat. No. 4,493,795.

Various diagnostic assays employing the above indicator moieties can be set up to test samples for Streptococcus pneumoniae. Exemplary assays are described in detail in Antibodies: A Laboratory Manual, Harlow and Lane (eds.), Cold Spring Harbor Laboratory Press, 1988. Representative examples of such assays include: countercurrent immuno-electrophoresis (CIEP), radioimmunoassays, radioimmuno-precipitations, enzyme-linked immuno-sorbent assays (ELISA), Western blot assays, dot blot assays, inhibition or competition assays, and sandwich assays, immunostick (dipstick) assays, simultaneous immunoassays, immunochromatographic assays, immunofiltration assays, latex bead agglutination assays, immunofluorescent assays, biosensor assays, and low-light detection assays (see e.g. also U.S. Pat. Nos. 4,376,110 and 4,486,530). An example of a suitable assay is an assay wherein a sample, e.g. a serum sample, is separated by electrophoresis and the polypeptide of interest, e.g. SEQ ID NO:16, is subsequently detected by Western blotting.

In one embodiment, the diagnostic kits of the present invention can be used in an “ELISA” format to detect the quantity of a preselected ligand in a fluid sample. “ELISA” refers to an enzyme-linked immunosorbent assay that employs an antibody or antigen bound to a solid phase and an enzyme-antigen or enzyme-antibody conjugate to detect and quantify the amount of an antigen present in a sample and is readily applicable to the present methods. Thus, in some embodiments, an indicator moiety of the present invention can be affixed to a solid matrix to form a solid support that comprises a package in the subject diagnostic systems. A reagent is typically affixed to a solid matrix by adsorption from an aqueous medium although other modes of affixation applicable to polypeptides, such as antibodies, can be used that are well known to those skilled in the art. Useful solid matrices are also well known in the art. Such materials are water insoluble and include the cross-linked dextran available under the trademark SEPHADEX from Pharmacia Fine Chemicals (Piscataway, N.J.); agarose; beads of polystyrene beads about 1 micron to about 5 millimetres in diameter available from Abbott Laboratories of North Chicago, Ill.; polyvinyl chloride, polystyrene, cross-linked polyacrylamide, nitrocellulose- or nylon-based webs such as sheets, strips or paddles; or tubes, plates or the wells of a microtiter plate such as those made from polystyrene or polyvinylchloride.

A further diagnostic method may utilise the multivalency of an antibody composition of one embodiment of this invention to cross-link ligands, thereby forming an aggregation of multiple ligands and polypeptides, producing a precipitable aggregate. This embodiment is comparable to the well known methods of immune precipitation. This embodiment comprises the steps of admixing a sample with a composition comprising an antibody of this invention to form a binding admixture under binding conditions, followed by a separation step to isolate the formed binding complexes. Typically, isolation is accomplished by centrifugation or filtration to remove the aggregate from the admixture. The presence of binding complexes indicates the presence of the preselected ligand to be detected.

### Binding Partners and Inhibitors of Polypeptides of the Invention

The surface-localisation of the polypeptides to which this invention relates makes them highly suitable as targets for binding partners, such as Inhibitors. Surface-located polypeptides of a pathogenic microorganism often interact with the host organism. Thus, any type of binding partner of a surface-located polypeptide may interfere with host-pathogen interaction. Binding partners thus often antagonise the pathogenicity (virulence) of a microorganism. A binding partner may also be an inhibitor of the polypeptide it binds.

Thus, in a further main aspect, the invention relates to methods for the identification of binding partners of the surface-located polypeptides set forth in SEQ ID NO:1-282. Such methods may be biochemical or cell-based.

### Biochemical Methods

In a main aspect, the invention relates to a method for identifying a binding partner of a polypeptide selected from the group consisting of SEQ ID NO:1-282, or a fragment thereof, comprising the steps of
a. providing a polypeptide selected from the group consisting of SEQ ID NO:1-282 preferably selected from the group consisting of SEQ ID NO:16, SEQ ID NO:10, SEQ ID NO:13, SEQ ID NO:20, SEQ ID NO:26, SEQ ID NO:28 and SEQ ID NO:33, most preferably SEQ ID NO:16,
   or
   a fragment thereof,
b. contacting said polypeptide or fragment with a putative binding partner, and
c. determining whether said putative binding partner is capable of binding to said polypeptide or fragment.
   In a preferred embodiment, said putative binding partner is a host-derived molecule.

In further preferred embodiments of the method, the polypeptide or fragment thereof is provided immobilised on a solid support, such as e.g. a column or microtiter plate, and, after the contacting step, it is determined whether or not the putative binding partner has bound to the solid support. Immobilisation of the polypeptide or fragment thereof may be through direct binding to the solid support, or through indirect binding e.g. using a specific antibody. In preferred embodiments, a washing step is performed between the contacting step and the determination step, in order to improve the specificity of detection. In further preferred embodiments, the putative binding partner is complexed with a detectable label. The putative partner may be labelled before the contacting takes place. Alternatively, labelling may also be performed after the contacting step. Furthermore, in some embodiments of this method, immobilisation may be performed after the polypeptide or fragment thereof has been bound to the binding partner. In preferred embodiments, the method is a screening method wherein the method is repeated for a plurality of putative binding partners. Suitable methods to determine binding are well-known in the art, and several of them have been referred to elsewhere herein.

In another aspect, a host-derived binding partner of a polypeptide selected from the group of SEQ ID NO:1-282, preferably SEQ ID NO:16 may be identified as follows: purified host membranes are electrophoretically separated, blotted over to a membrane and incubated with the polypeptide of interest or fragment thereof. Binding can then be detected using antibodies specific for the polypeptide of interest or fragment thereof. The host binding partner to which the polypeptide or fragment thereof has bound can subsequently be identified by elution from the blot and subsequent analysis by mass spectrometry, or by any other technique known in the art.

If the binding partner of a surface-located polypeptide of a pathogenic organism is a host-derived molecule, then such an interaction between the surface-located polypeptide and the host may be important for the virulence of the bacterium. Compounds that interfere with the interaction of the surface-located polypeptide and the host-derived binding partner may thus be suitable for prevention or treatment of Streptococcus pneumoniae infections. Accordingly, another method of the invention relates to a method of identifying an inhibitor of the interaction of any of the surface-located Streptococcus pneumoniae polypeptides of SEQ ID NO:1-282 with a host-derived binding partner comprising the steps of:
a. providing any of the polypeptides of SEQ ID NO:1-282, preferably selected from the group consisting of SEQ ID NO:16, SEQ ID NO:10, SEQ ID NO:13, SEQ ID NO:20, SEQ ID NO:26, SEQ ID NO:28 and SEQ ID NO:33, most preferably SEQ ID NO:16
   or
   a fragment thereof,
b. providing a host-derived binding partner of said polypeptide (identified as described above or by any other method),
c. contacting said polypeptide with said host-derived binding partner in the absence of a putative inhibitor of said interaction,
d. contacting said polypeptide with said host-derived binding partner in the presence of said putative inhibitor,
   and
e. determining whether the strength of the binding of said polypeptide to said host-derived binding partner resulting from step d. is reduced as compared to that resulting from step c.
   In some embodiments, step c. and d. may be performed in two different sample compartments. In other embodiments, step d. may be performed by adding the putative inhibitor to the mixture of step c. In preferred embodiments, the method is repeated for a plurality of putative inhibitors.

Of particular interest are binding partners that inhibit an activity of a surface-located polypeptide. Such activity may be enzymatic activity, transport activity, or any type of other biochemical or cellular activity, preferably enzymatic activity.

Preferred host-derived binding partners are host polypeptides and host lipids. Binding may e.g. be determined as described by Szymanski and Armstrong (1996) Infect. Immun. 64:3467-3474.

In preferred embodiments of the above described biochemical methods, the binding between the binding partner and the surface-located polypeptide or fragment thereof has a dissociation constant or Kd of less than 5×10⁻⁶M, such as less than 10⁻⁶M, e.g. less than 5×10⁻⁷M, such as less than 10⁻⁷M, e.g. less than 5×10⁻⁸M, such as less than 10⁻⁸M, e.g. less than 5×10⁻⁹M, such as less than 10⁻⁹M, e.g. less than 5×10⁻¹⁰M, such as less than 10⁻¹⁰ M, e.g. less than 5×10⁻¹¹M, such as less than 10⁻¹¹M, e.g. less than 5×10⁻¹²M, such as less than 10⁻¹²M. Dissociation constants can e.g. be determined by surface plasmon resonance analysis.

### Cell-Based Methods

Reducing the level of a surface-located polypeptide, by deletion or disruption of the structural gene for it or by down-regulating gene expression (see below), may affect a bacterial cell. The cell may become more sensitive to cytotoxic compounds. Especially for surface-located polypeptides, a reduction of their level may affect the function of the cell's exterior parts, such as the membrane or cell wall, in preventing compounds of entering the cell. Thus, reduction of the level of an surface-located polypeptide can make a cell more ‘permeable’ for various compounds.

Thus, an aspect of the present invention relates to a method for identifying a compound with antibacterial activity against Streptococcus pneumoniae comprising the steps of
a. providing a sensitised cell which has a reduced level of any of the polypeptides of SEQ ID NO:1-282, preferably selected from the group consisting of SEQ ID NO:16, SEQ ID NO:10, SEQ ID NO:13, SEQ ID NO:20, SEQ ID NO:26, SEQ ID NO:28 and SEQ ID NO:33, most preferably SEQ ID NO:16
   and
b. determining the sensitivity of said cell to a putative antibacterial compound, for instance by a growth assay.

Preferably, the method is a screening method wherein the procedure is repeated for a plurality of putative antibacterial compounds. Preferred putative antibacterial compounds are ones that do not pass through the membrane of a wild-type Streptococcus pneumoniae cell.

The rationale behind this approach is that a cell with a lower level of the surface-located polypeptide will exhibit increased sensitivity to cytotoxic compounds, allowing identification of antibacterial compounds with low potency that are missed when using wild-type cells for the assay. Compounds identified by this method will be often need to be modified in order to improve potency. This can be done by chemical modification.

Inhibition of the activity of a surface-located polypeptide may affect the viability (i.e. survival, growth and/or proliferation) of the bacterium. Of particular interest is inhibition of surface-located polypeptides that are essential for viability of Streptococcus pneumoniae. Methods for testing essentiality of a Streptococcus pneumoniae gene have been described in the prior art, e.g. in Chan et al. (2002) J. Bacteriol 185:2051-2058 and Thanassi et al. (2002) Nucleic Acid Res. 30:3152-31-62. Inhibitors of essential surface-located polypeptides may not need to enter the bacterial cell to be able to affect its viability. Thus, generally fewer requirements are posed on the structure of an inhibitor of an essential surface-located target polypeptide than on an inhibitor of an intracellular target, to be effective as an antibacterial agent.

Accordingly, the invention relates to a method for identifying an inhibitor of a polypeptide selected from the group consisting of SEQ ID NO:1-282, comprising the steps of
a. providing two cells which differ in the level of any of the polypeptides of SEQ ID NO:1-282, preferably selected from the group consisting of SEQ ID NO:16, SEQ ID NO:10, SEQ ID NO:13, SEQ ID NO:20, SEQ ID NO:26, SEQ ID NO:28 and SEQ ID NO:33, most preferably SEQ ID NO:16
b. determining the sensitivity of said cells to a putative inhibitor, for instance by a growth assay, and
c. determining whether said two cells are differently affected by the presence of said putative inhibitor.
   Preferably, the method is repeated for a plurality of putative inhibitors. Preferred inhibitors are ones that do not pass through the membrane of a Streptococcus pneumoniae cell.

The rationale behind this approach is that the viability of a cell with a lower activity of the essential polypeptide will be more affected by an inhibitor of the polypeptide than the viability of the cell with a higher level. If the two cells are differently affected, this is an indication that the inhibitor acts on the target or at least in the same biochemical pathway.

In some embodiments of the method, the two cells with different activity of the polypeptide of interest are a wild-type cell (or other cell with wild-type activity of the gene of interest) and a sensitised cell with a reduced activity of the polypeptide of interest. In some embodiments, the different or reduced level in the sensitised cell can be a different or reduced expression level of the gene of interest (resulting in a different or reduced copy number of the polypeptide). This can be accomplished by putting the gene under control of a regulatable promoter or by regulatable expression of an antisense RNA which inhibits translation of an mRNA encoding the essential polypeptide. In other embodiments, the different or reduced activity can be a different or reduced activity of the polypeptide of interest, e.g. due to a mutation, such as a temperature-sensitive mutation.

Suitable ways of generating sensitised bacterial cells and of using these in screening for inhibitors have been described in WO 02/077183. Sensitised cells may be obtained by growing a conditional-expression Streptococcus pneumoniae mutant strain in the presence of a concentration of inducer or repressor or other conditions which provide a level of a gene product required for bacterial viability such that the presence or absence of its function becomes a rate-determining step for viability. The sub-lethal expression of the target gene may be such that growth inhibition is at least about 10%, such as at least about 25%, e.g. at least about 50%, such as at least about 75%, e.g. at least 90%, such as at least 95%.

In another embodiment of the cell-based assays of the present invention, sensitised cells are obtained by reduction of the level activity of a polypeptide required for bacterial viability using a mutation, such as a temperature-sensitive mutation, in the polypeptide. Growing such cells at an intermediate temperature between the permissive and restrictive temperatures produces cells with reduced activity of the gene product. It will be appreciated that the above method may be performed with any mutation which reduces but does not eliminate the activity or level of the gene product which is required for bacterial viability. This approach may also be combined with the conditional-expression approach. In this combined approach, cells are created in which there is a temperature-sensitive mutation in the gene of interest and in which this gene is also conditionally-expressed.

When screening for inhibitors of an essential polypeptide, growth inhibition can be measured by directly comparing the amount of growth, measured by the optical density of the culture relative to uninoculated growth medium, in an experimental sample with that of a control sample. Alternative methods for assaying cell proliferation include measuring green fluorescent protein (GFP) reporter, construct emissions, various enzymatic activity assays, and other methods well known in the art. Other parameters used to measure viability include e.g. colony forming units. The above method may be performed in solid phase, liquid phase, a combination of the two preceding media, or in vivo. Multiple compounds may be transferred to agar plates and simultaneously tested using automated and semi-automated equipment. Cell-based assays of the present invention are capable of detecting compounds exhibiting low or moderate potency against the target molecule of interest because such compounds are substantially more potent on sensitised cells than on non-sensitised cells. The effect may be such that a test compound may be two to several times more potent, e.g. at least 10 times more potent, such as at least 20 times more potent, e.g. at least 50 times more potent, such as at least 100 times more potent, e.g. at least 1000 times more potent, or even more than 1000 times more potent when tested on the sensitised cells as compared to non-sensitised cells.

A mutant Streptococcus pneumoniae strain that overexpresses a surface-located polypeptide can also be used to identify a compound that inhibits such a polypeptide. If the compound is cytotoxic, overexpression of the target polypeptide can make cells more resistant. Thus, the invention also relates to a method for finding an inhibitor of any of the surface-located Streptococcus pneumoniae polypeptides of SEQ ID NO:1-282 comprising the steps of
a. providing two cells which differ in the activity of any of the surface-located Streptococcus pneumoniae polypeptides of SEQ ID NO:1-282, preferably selected from the group consisting of SEQ ID NO:16, SEQ ID NO:10, SEQ ID NO:13, SEQ ID NO:20, SEQ ID NO:26, SEQ ID NO:28 and SEQ ID NO:33, most preferably SEQ ID NO:16
   wherein one cell contains a substantially wild-type copy number of said polypeptide and the other cell contains higher than wild-type copy number of said polypeptide,
b. determining the sensitivity of said cells to a putative inhibitor, for instance by a growth assay, and
c. determining whether or not said two cells are differently affected by the presence of said putative inhibitor.
   Overexpression may be achieved using strong promoters or by introducing multiple copies of the structural gene for a surface-located polypeptide. As also overexpression of polypeptides that are not the cellular target of an inhibitor can make cells resistance to an inhibitor, inhibition of the target polypeptide of interest by a putative inhibitor will need to be verified by other means, such as e.g. a biochemical assay.

In addition to inhibitors of a biochemical or other cellular activity of a surface-located polypeptide, the cellular methods described above can be used to identify compounds that reduce the expression level of a target, and thereby its copy number, e.g. by interfering with gene regulation.

In preferred embodiments of the any of the cell-based- or biochemical methods for finding binding partners or inhibitors, the method is repeated for a plurality of candidate compounds.

In a further aspect, the invention relates to the mutant Streptococcus pneumoniae strains used in the cell-based methods described herein, such as strains in which the gene encoding the surface-located polypeptide is placed under the control of a heterologous regulatable promoter, strains carrying temperature-sensitive alleles of the surface-located polypeptides, and strains overexpressing the surface-located polypeptides.

Other methods of interfering with bacterial growth by targeting surface-located polypeptides, such as any of the polypeptides of SEQ ID NO:1-282 include suppression of gene expression using specific antisense molecules, such antisense RNA or DNA, and using ribozyme molecules specific for mRNA encoding the essential surface-located polypeptides.

### Example 1

### Strategy

The experimental steps in the project are as follows: Surface proteins were isolated by either high pH elution or by mutanolysin digestion. Isolated of surface proteins were identified by three complementary mass spectrometric based strategies: 1) 2-D SDS PAGE, 2) 1D SDS PAGE and 3) In-solution digest. All three strategies include protein identification by mass spectrometry analysis. The surface identified proteins are cloned into an E. coli expression vector. The expressed recombinant proteins are purified and used to immunise mice to verify immunogenicity of the antigens. The immunised mice are used in challenge studies in which the mice are challenged with Streptococcus pneumoniae and protection against disease and/or death is monitored.

### Mice:

Six-weeks-old BALB/c mice were housed under specific-pathogen-free conditions and given sterile food and water ad libitum.

### Bacteria:

Escherichia coli Top10 (Invitrogen) was used as the host for routine plasmid cloning. Recombinant proteins were expressed in E. coli BL21/(DE3) (Invitrogen). E. coli were cultured in Luria broth supplemented with antibiotics. Virulent S. pneumoniae strain D39 (serotype 2, purchased by Dr. M. Trombe, CICT, Toulouse, France) was used for proteomics, challenge experiments and as a source of genomic DNA for PCR amplification experiments. Clinical isolates of S. pneumoniae, including 40 serotypes responsible for the majority of pneumococcal infections were selected and purchased from the WHO Collaborating Centre for Reference and Research on Pneumococci in Copenhagen, Denmark. S. pneumoniae were routinely grown on blood agar plates (Difco).

Isolation of S. pneumoniae Cell Envelope Fraction:

Mutanolysin digestion of pneumococcal cell wall. Bacteria were grown overnight on blood agar plates, harvested into phosphate-buffered saline (PBS) containing 20% sucrose and pelleted by centrifugation at 6000 g for 10 minutes. The pellet was resuspended in 0.5 ml of osmotic digestion buffer (20% sucrose in 20 mM Tris-HCl, pH 7.0, 10 mM MgCl₂, protease inhibitor cocktail and 100 U/ml mutanolysin (Sigma) per plate. Enzymatic digestion was allowed to proceed for 1-2 h at 37° C. The intact protoplasts were removed by centrifugation at 7,000×g for 15 min. The supernatant was collected, acetone precipitated and analysed using mass spectrometry based techniques.

High pH elution of surface proteins. Bacteria were grown overnight on blood agar plates, harvested into PBS containing 20% sucrose and pelleted by centrifugation at 6000 g for 10 minutes. The pellet was resuspended in PBS containing 20% sucrose and centrifuged again as above. Then bacteria were resuspended in 2 ml of 50 mM glycine-NaOH (pH 12) containing 20% sucrose per plate. Alkali extraction of cell surface proteins was allowed to proceed for 30 minutes at room temperature with gentle shaking. The suspension was centrifuged at 15,000 g for 20 min, the supernatant was collected, adjusted to pH 7 with 1 M HCl, acetone precipitated and analysed by 1-D and 2-D gel electrophoresis.

### Surface Protein Identification:

The complex mixture of proteins obtained after surface extraction was analysed by three complementary strategies all based on mass spectrometry: 2D gel based strategy, 1D gel based strategy and In-solution digest strategy.

2-D gel based strategy (2D-gel MALDI-TOF/TOF): Two-dimensional gel electrophoresis was performed either on the Ettan Dalt 2 system (Amersham Biosciences) or on the Novex NuPage system (Invitrogen) according to the manual provided with the gel system. In brief: First dimension runs were performed on either 7 cm or 24 cm pre-cast IPG strips (pH range 3-10 or 4-7) using the Ettan IPGphor isoelectric focusing system (Amersham Biosciences) according to the manufacturer's instructions. Isofocusing was performed at the following conditions: 7 cm strips: 8000 Vh, 24 cm strips: 52000 Vh. The second dimension was performed using pre-cast 12.5% gels (Amersham Biosciences) at 5 W per gel for 15 min then total 170 W for 4-6 hours for 24 cm strips. The 7 cm strips were run on the Novex NuPage system (Invitrogen) using pre-cast 4-12% gels (Invitrogen) at 200 volts for 40 minutes. Gels were silver stained according to a modified method described originally by Mortz et al. (2001) Proteomics 1(11), 1359-1363, and spots for mass spec analysis were picked using the Ettan Spot Picker from Amersham according to the manufacturer instructions.

Specific protein spots were spot-picked, and placed in Milli-Q water. These gel plugs were washed in 50 mM NH₄HCO₃/50% ethanol and dehydrated by incubation in 96% ethanol. Reduction and alkylation was performed by incubating in reducing solution (10 mM DTT, 50 mM NH₄HCO₃) at 56° C. followed by a room temperature incubation in alkylation solution (55 mM iodoacetamide, 50 mM NH₄HCO₃) in the dark. Two cycles of washing and dehydration were then performed prior to the addition of 5 ul trypsin solution (12.5 ng/ul Promega trypsin in 50 mM NH₄HCO₃, 10% Acetonitrile). Then an additional amount of sodium bicarbonate solution was added and the digests were incubated overnight at 37° C. Trifluoroacetic acid was added to the overnight digest followed by incubation with shaking.

Parts of the extract were used in MALDI-TOF peptide mass fingerprint and MALDI-TOF/TOF analysis (Ultraflex, Bruker Daltonics, Germany) and the peak-lists were used in database searching against a specific S. pneumoniae database. The Mascot search program and scoring algorithm (Matrix Science, UK) was used in database searching. Peptide mass tolerance was set to 60 ppm and 0.7 Da, respectively. Search parameters were adjusted to include oxidation of Met, the addition of Carbamidomethyl groups to Cys, and trypsin was allowed to miss one cleavage site per peptide.

1D gel based strategy (GeLC-MS/MS): One-dimensional gel electrophoresis was performed on a Novex NuPage system (Invitrogen) according to the manual provided with the gel system. In brief we used, size 8 cm×8 cm, 1 mm thick pre-cast 12% bis-tris gels (Invitrogen) at 200 volts for 60 minutes in NuPage-MOPS-SDS running buffer. Gels were silver stained according to a modified method described originally by Mortz et al. (2001) Proteomics 1(11), 1359-1363. Whole lanes were cut out with razorblade in 0.5 cm gel slices. The gel slices were digested as described under 2D gel bases strategy, but the amount of trypsin was 20 ul trypsin solution (12.5 ng/ul Promega trypsin in 50 mM NH₄HCO₃, 10% Acetonitrile).

The extracts was analysed on a LC-MS/MS (Waters Cap-LC and Micromass Ultima TOF MS). Each extract was submitted to a 115 minutes LC-MS/MS analysis. The peak lists generated from the fragmented peptides were used in database searching against a specific Streptococcus pneumoniae database. The Mascot search program and scoring algorithm (Matrix Science, UK) was used in database searching. Peptide mass tolerance was set to 200 ppm and 0.4 Da for fragment ions. Search parameters were adjusted to include oxidation of Met, the addition of Carbamidomethyl groups to Cys, and trypsin was allowed to miss one cleavage site per peptide.

In-solution based strategy (ISD-MS/MS): The protein mixture was resuspended in 50 mM NH₄HCO₃, 10% Acetonitrile. Trypsin solution (50 μl) (12.5 ng/μl Promega trypsin in 50 mM NH₄HCO₃, 10% Acetonitrile) was added and the mixture was incubated overnight at 37° C. The digestion was stopped by acidification with TFA (final conc. 1%) and analysed by LC-MS/MS as described under 1D-based strategy. Database search was also performed as described under 1D-based strategy.

Detection of Genes for Protein Vaccine Candidates in Different S. pneumoniae Strains:

PCR amplification was used to demonstrate the presence of genes encoding antigens listed in clinical isolates of S. pneumoniae. For this purpose cells were grown on blood agar and diluted in PBS. Genomic DNAs were prepared from 40 pneumococcal strains by heating (95° C. for 5 min) and aliquots were used as templates for PCR amplification with Taq polymerase (Qiagen) with gene specific primers. Amplification products were electrophoresed through 1% agarose gels and visualized by staining with ethidium bromide (0.5 μg/ml).

### Reverse Transcription Polymerase Chain Reaction (RT-PCR):

A BALB/C mouse was infected with S. pneumoniae D39 as described below under Pneumococcal challenge. After 1 day of infection this mouse was sacrificed and the spleen was extracted and divided in two pieces. For isolation of intact total bacteria RNA from tissue, one half the organ was frozen quickly in liquid nitrogen and stored at −80° C. before RNA isolation. The other half of each organ was tested for bacteria using blood agar plates before RNA isolation (data not shown). Total RNA was isolated from animal tissue containing S. pneumoniae with the Rneasy Kit (Qiagen, Hilden). First-strand cDNA synthesis was performed with total RNA and the iScript Kit (Biorad). 1 μl cDNA were used for the subsequent PCR-step with gene specific primers.

### Production of Recombinant Vaccines (rec. vac.):

The production of recombinant vaccine was achieved by PCR amplification of pneumococcal genes, with subsequent cloning and expression of the genes in E. coli. Oligonucleotide primers used in PCR amplification experiments were all purchased from MWG, Germany. Pneumococcal genes used for protein expression were amplified from genomic DNA of S. pneumoniae strain D39 by using the high-fidelity thermostable DNA polymerase, Platinum Pfx (Life Technologies). The coding sequence was amplified with primers containing the start-codon but excluding the stop-codon of the open reading frame. The coding sequences used for protein expression were cloned into plasmid pET101 (invitrogen) using directional Topo cloning kit, with E. coli Top10 as the bacterial host. A plasmid-encoded C-terminal polyhistidine tag flanks each recombinant protein. For recombinant protein expression, each recombinant pET101 plasmid was subcloned into the E. coli strain BL21 (DE3). Recombinant protein expression was initiated by induction with IPTG (isopropyl-β-D-thiogalactopyranoside), and proteins were purified from the soluble fraction of recombinant E. coli lysates by using metal affinity chromatography resin and buffers (invitrogen), according to the manufacturer's instructions. Protein concentrations were estimated by using the BCA test (Hercules, Calif.). The recombinant proteins were dialyzed against PBS (Millipore) and stored at −80° C. Recombinant Proteins were identified with MALDI MS technology after purification.

### Western Blot Analysis:

Purified proteins were separated on one-dimensional (ca. 20 μg protein) SDS-PAGE and transferred to PVDF membranes. The immunological detection of immobilized proteins was performed after manufactures instructions (invitrogen). Patient sera were kindly provided by Dr. M. Trombe (CICT, Toulouse, France). We used a dilution of 1:100 with single and pooled patient sera for immunodetection. The dilution of the secondary antibody was 1:5000.

### Pneumococcal Challenge of Actively Immunized Mice:

BALB/C mice were used in challenge experiments (10 mice per group). For an antigen specific vaccination mice were primed with 25 μg of each antigen containing the ALUM adjuvant (100 μg) on day zero. Animals were boosted with the same antigen concentration on day 14 and day 28. We used untreated BALB/c mice and mice treated with an unrelated cellulose-binding domain CBD (sigma) as negative controls. All vaccines were administered subcutaneously (s.c.). All mice were bled on days 0, 21 and 36 and challenged on day 35 with S. pneumoniae D39. Individual sera from each immunized mouse were tested for the presence of specific antibodies prior to challenge. Virulent S. pneumoniae (D39) grown on blood agar plates was prepared for challenge via the intra peritoneal (i.p.) route in actively immunized mice or control groups. For challenge infections, mice were injected i.p. with approximately 10⁷ CFU per mouse of virulent S. pneumoniae strain D39 suspended in PBS. The actual number of CFU administered was determined retrospectively by plating serial dilutions of the inocula on blood agar. The survival of mice was monitored for 7 days, at which time the experiments were terminated.

### ELISA for Detection of Immunoglobulin G (IgG) Mouse:

Elisa assays were developed for detection of antigen specific IgG in mouse sera at day 0, day 21 and day 35 after vaccination. Different ELISA plates were coated with recombinant vaccines (2 μg) and whole cell lysates (2 μg) of S. pneumoniae D39. Two fold serial dilutions were made from mouse sera as a primary antibody. The dilution of the secondary antibody (goat anti mouse IgG, horse radish peroxidase conjugated) was 1:5000. OPD substrate was used for the color development. Plates were read at 492 nm. Results of IgG status at day 21 and day 35 after vaccination were compared with IgG status at day 0 of vaccination.

### Results:

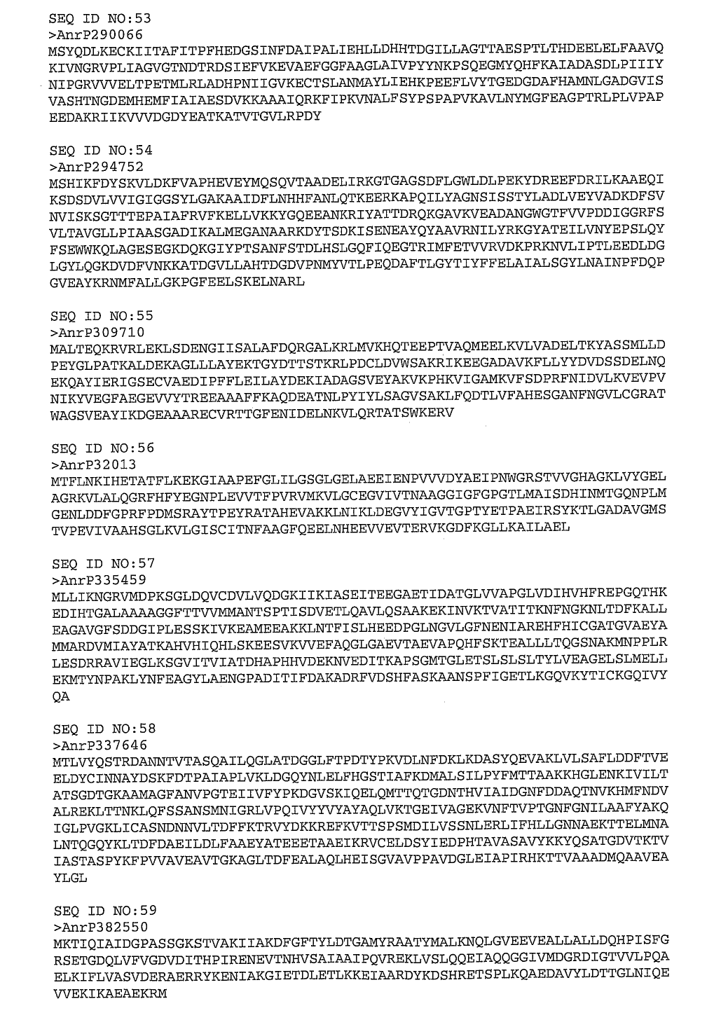

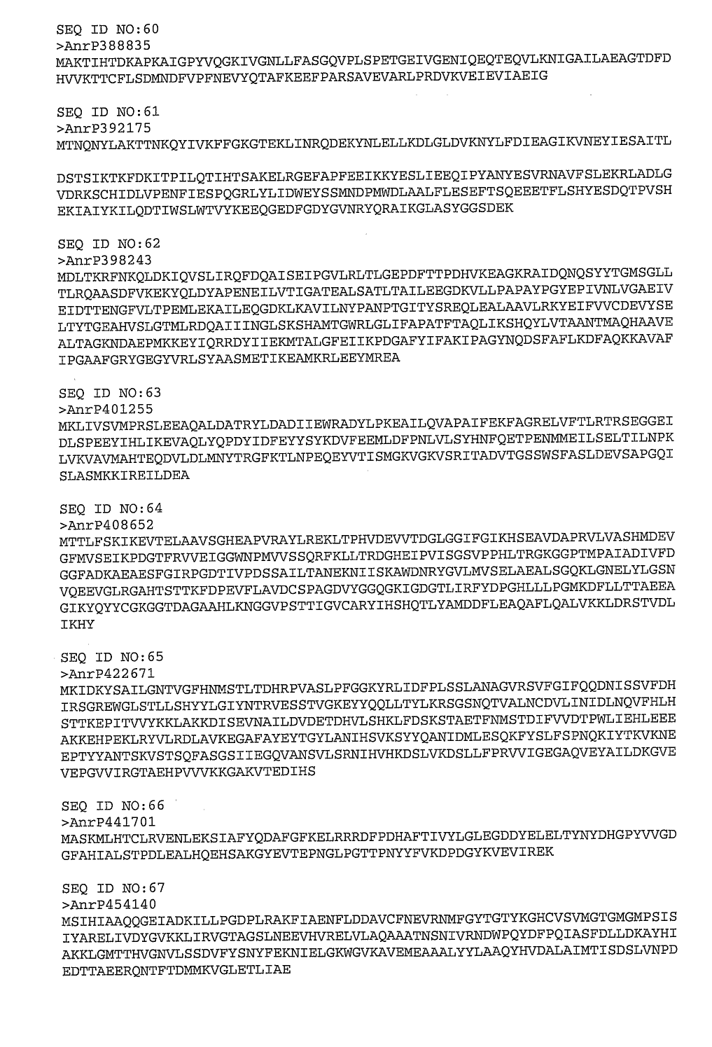

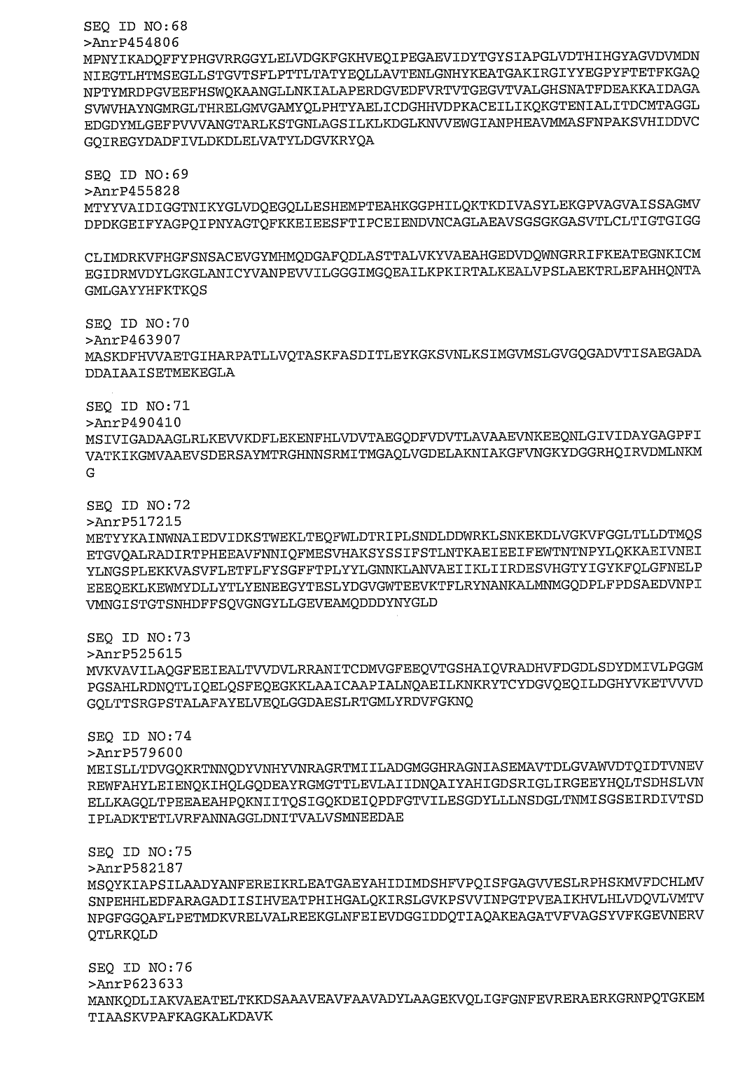

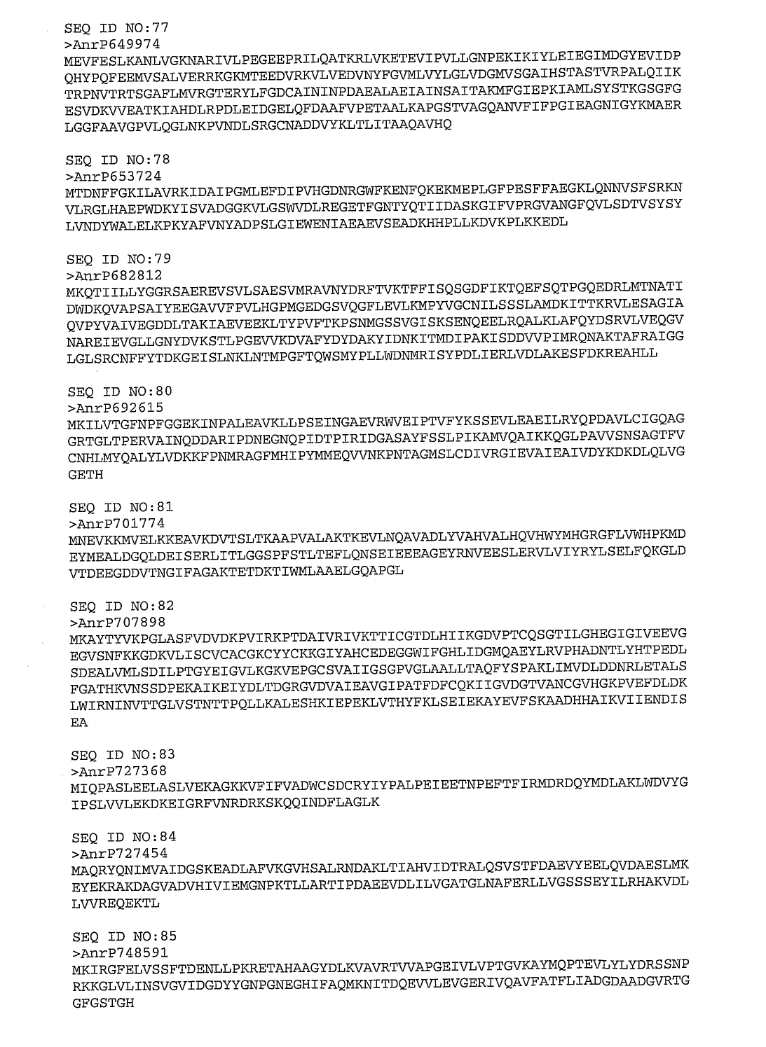

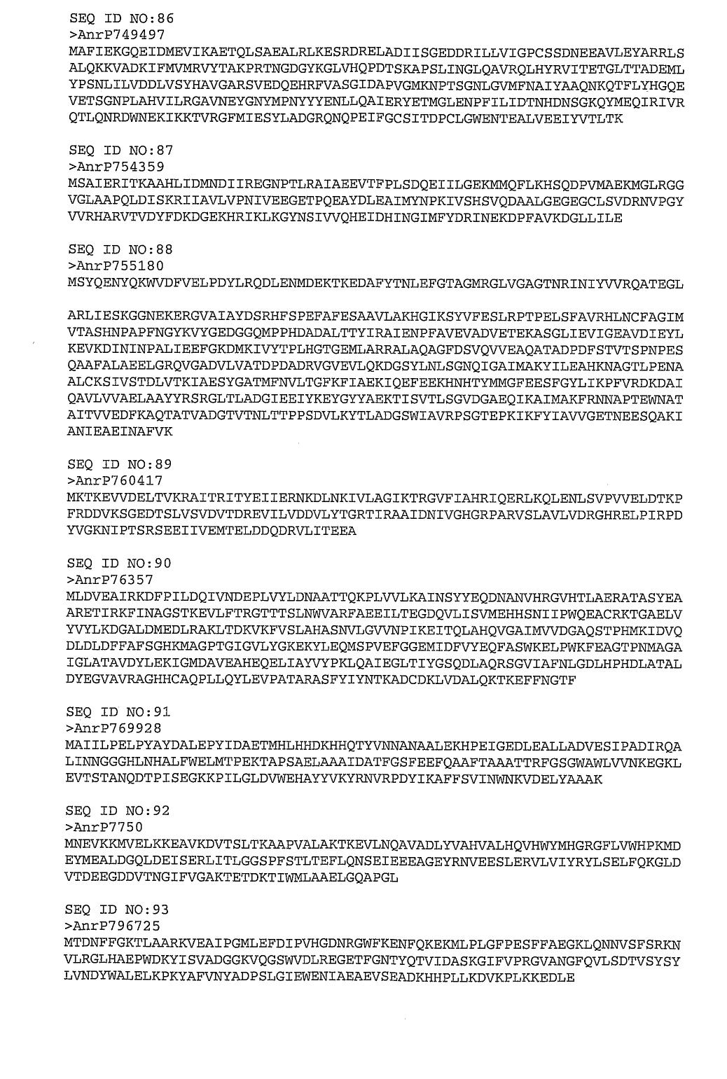

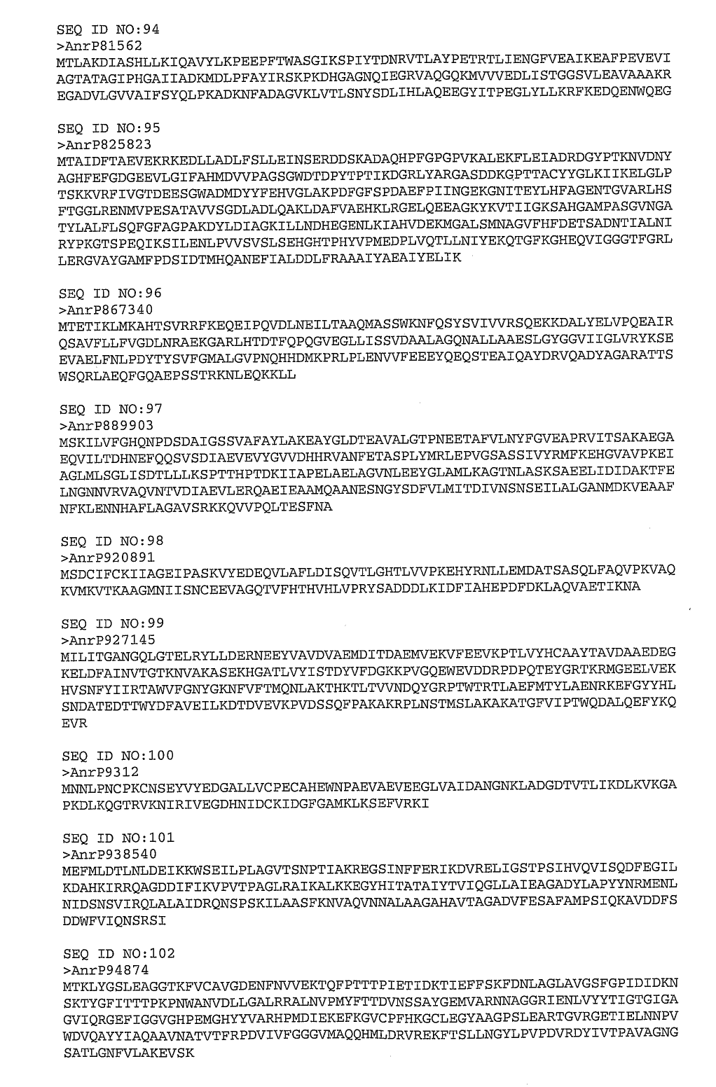

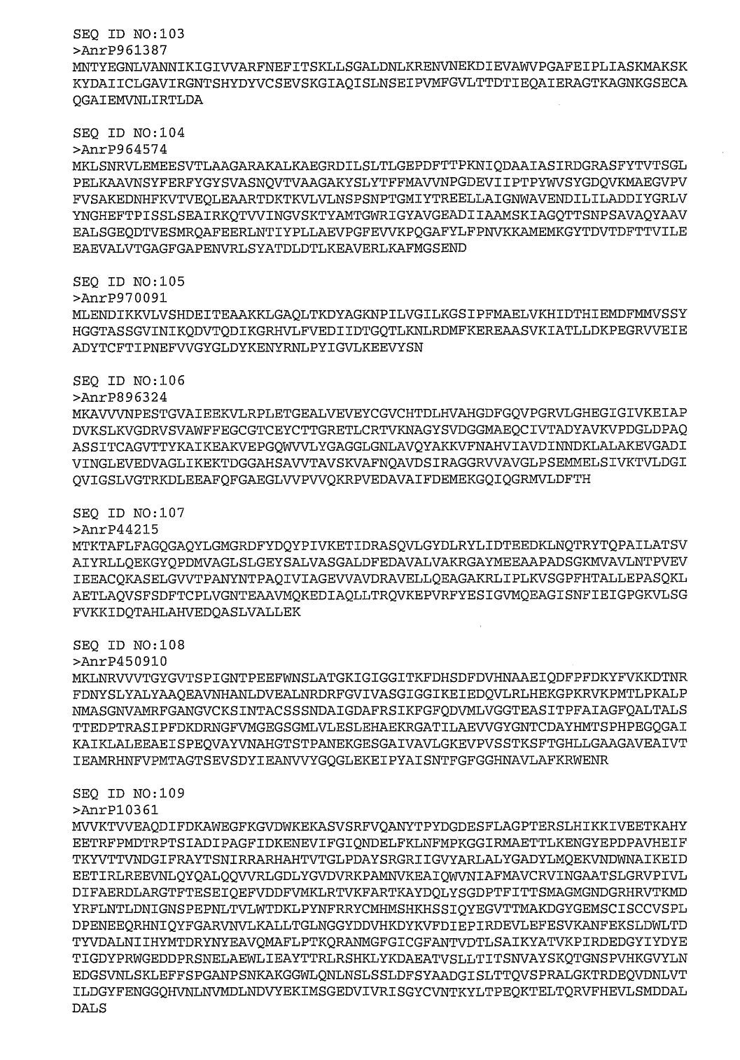

We identified 282 different polypeptides in mutanolysin created cell-surface fractions of S. pneumoniae. Sequences of the identified polypeptides are given in FIG. 2. The methods that were employed identifies polypeptides that are expressed at a relatively high level. We used three different strategies for the detection of isolated S. pneumoniae surface proteins: a) a 2-D gel based strategy, b) a strategy with 1-D gels and LC_MS/MS and c) an in-solution based MS strategy. We selected ORFs of identified surface proteins for characterization. ORFs of genes were amplified with PCR and cloned directional into pET101. Recombinant proteins were expressed in E. coli as described in materials and methods. Four recombinant proteins were selected for further studies (table 3):

| | AnrP number | |
|---|---|---|
| Group# | (antigen#, short#) | Description |
| 1 | 230653 (1, 653) | hydrolase, putative [Streptococcus |
| | | pneumoniae TI; ca. 32 kDa (rec. vac.) |
| 2 | 516029 (2, 029) | lipoate-protein ligase, putative |
| | | [Streptococcus pneumoniae TIGR4]; |
| | | ca. 40 kDa (rec. vac.) |
| 3 | 800607 (3, 607) | ATP-dependent C1p protease, proteolytic |
| | | subunit; ca. 24 kDa (rec. vac.) |
| 4 | 944060 (4, 060) | autoinducer-2 production protein; |
| | | 21 kDa (rec. vac.) |

A PCR investigation demonstrated that genes of each of these 4 selected proteins are presented in 40 different serotypes of S. pneumoniae (table 4).

TABLE 4
PCR with different S. pneumoniae serotypes and specific primers for
following genes (+ = detected; − = not detected)

| Serotype | AnrP230653 | AnrP516029 | AnrP800607 | AnrP944060 |
|---|---|---|---|---|
| 1 | + | + | + | + |
| 2 | + | + | + | + |
| 3 | + | + | + | + |
| 4 | + | + | + | + |
| 5 | + | + | + | + |
| 6a | + | + | + | + |
| 6b | + | + | + | + |
| 7f | + | + | + | + |
| 7c | + | + | + | + |
| 8 | + | + | + | + |
| 9n | + | + | + | + |
| 9v | + | + | + | + |
| 9a | + | + | + | + |
| 10a | + | + | + | + |
| 10b | + | + | + | + |
| 11a | + | + | + | + |
| 12f | + | + | + | + |
| 13 | + | + | + | + |
| 14 | + | + | + | + |
| 15b | + | + | + | + |
| 15c | + | + | + | + |
| 16f | + | + | + | + |
| 17f | + | + | + | + |
| 18b | + | + | + | + |
| 18c | + | + | + | + |
| 19a | + | + | + | + |
| 19f | + | + | + | + |
| 20 | + | + | + | + |
| 22f | + | + | + | + |
| 23a | + | + | + | + |
| 23f | + | + | + | + |
| 28f | + | + | + | + |
| 31 | + | + | + | + |
| 31f | + | + | + | + |
| 32 | + | + | + | + |
| 33f | + | + | + | + |
| 34 | + | + | + | + |
| 35b | + | + | + | + |
| 35f | + | + | + | + |
| 38 | + | + | + | + |

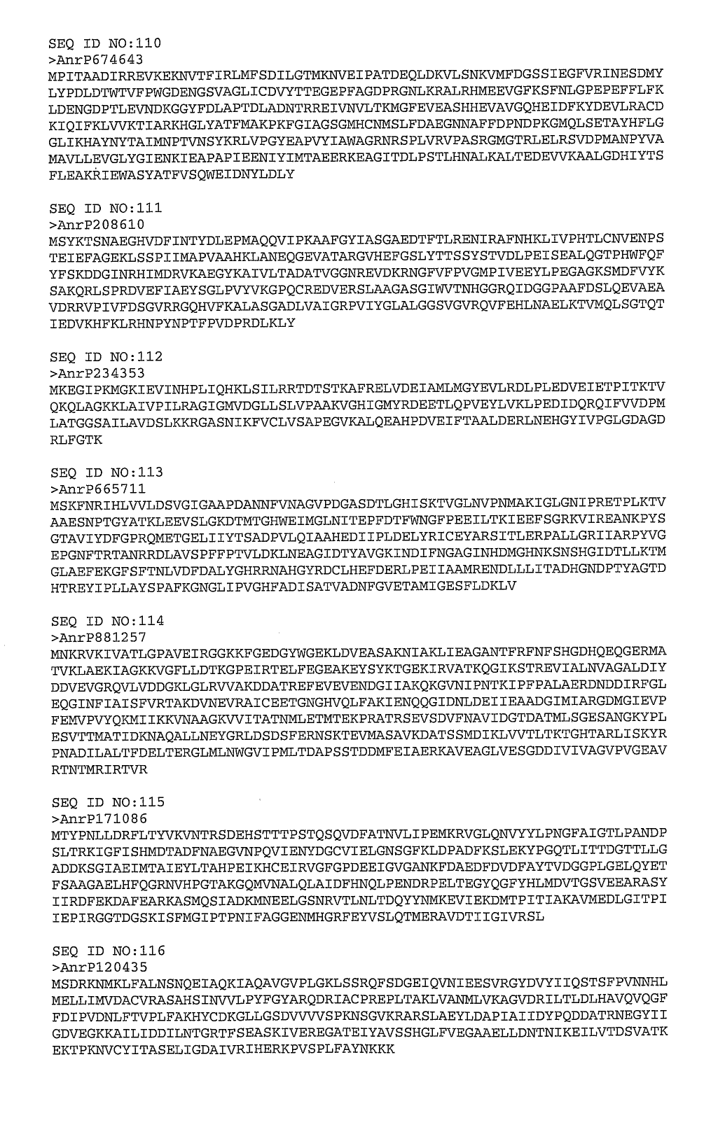

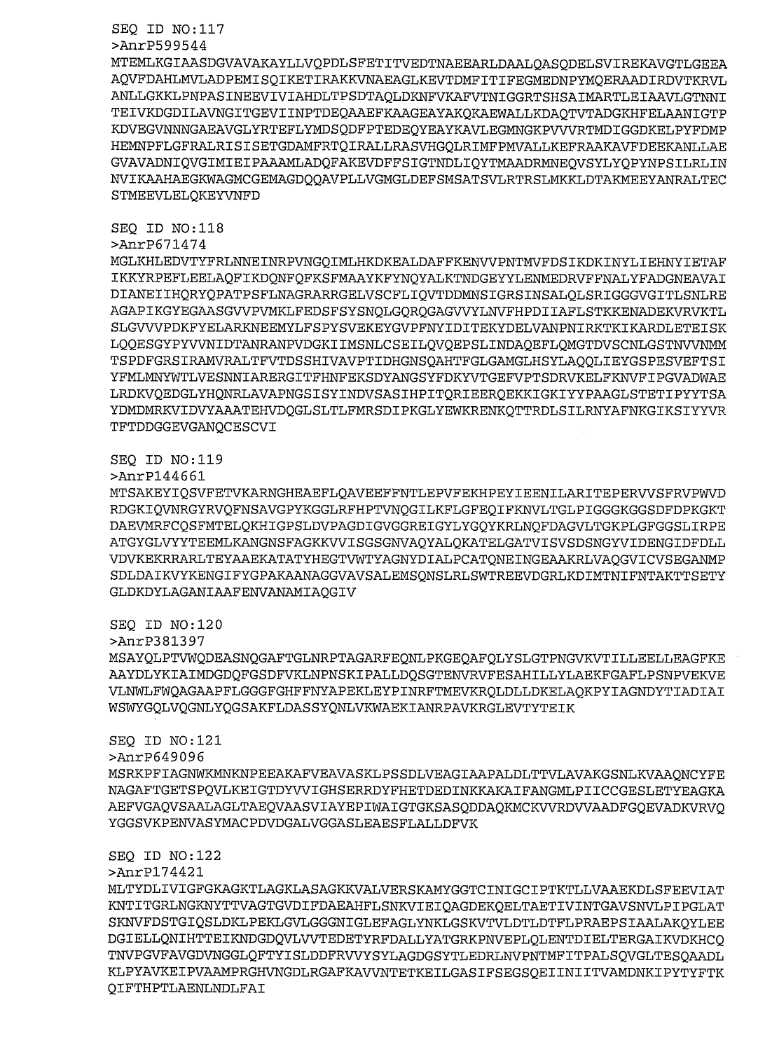

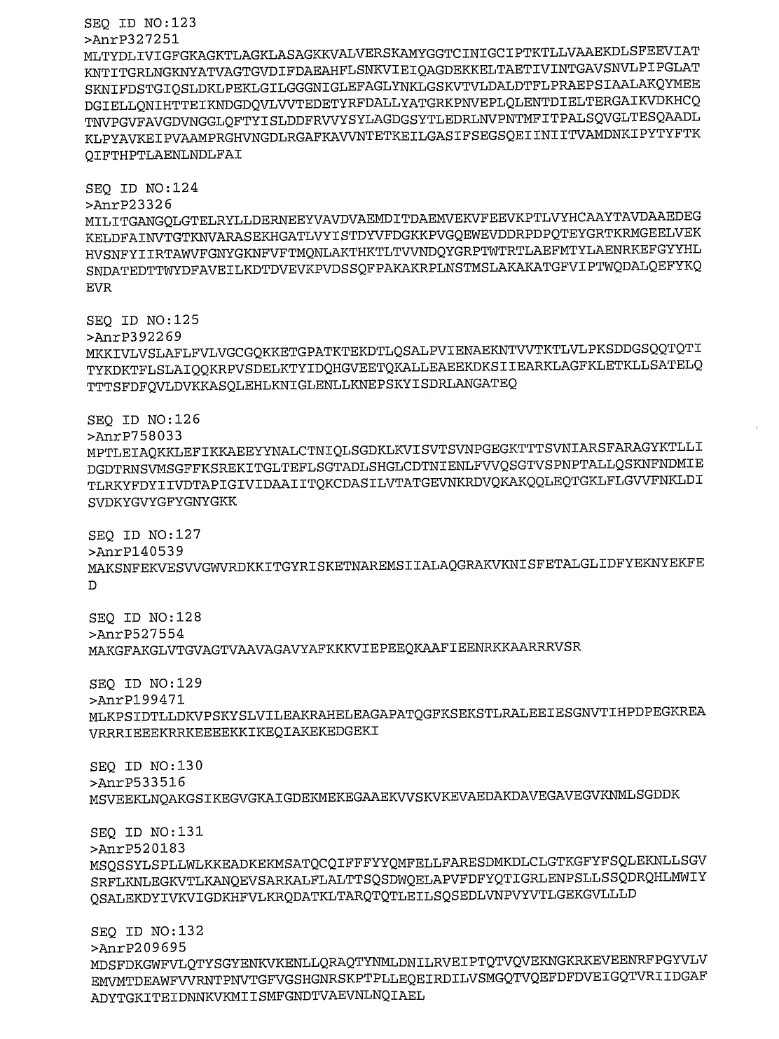

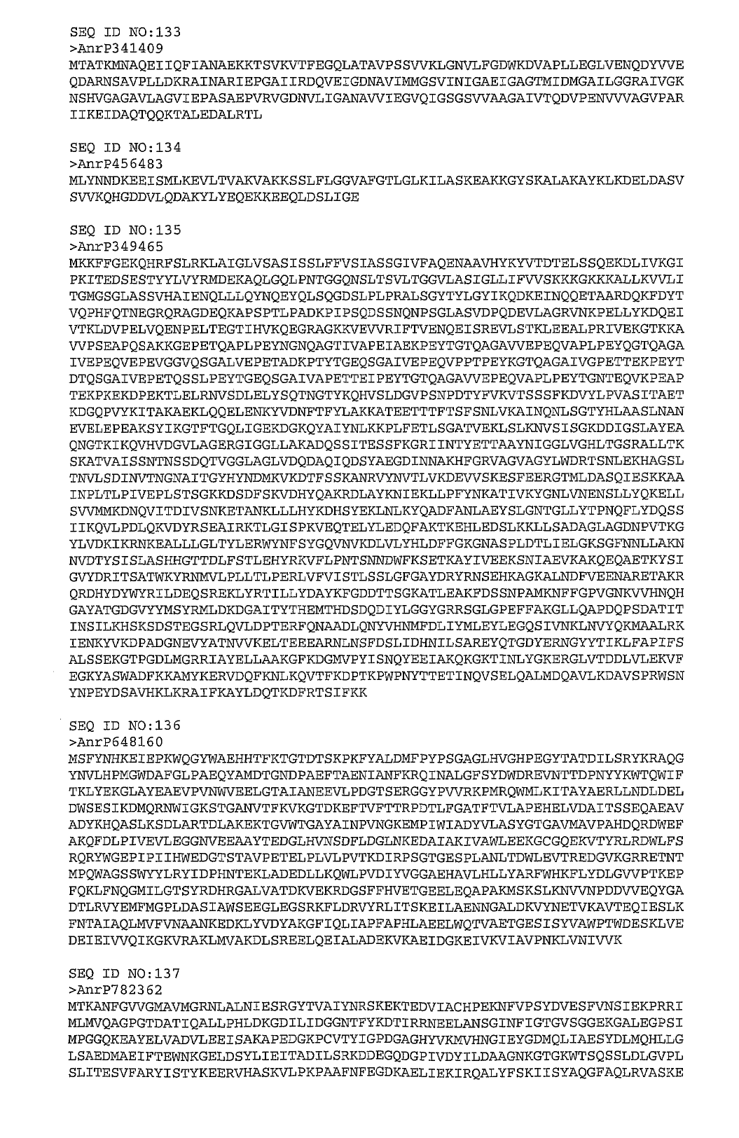

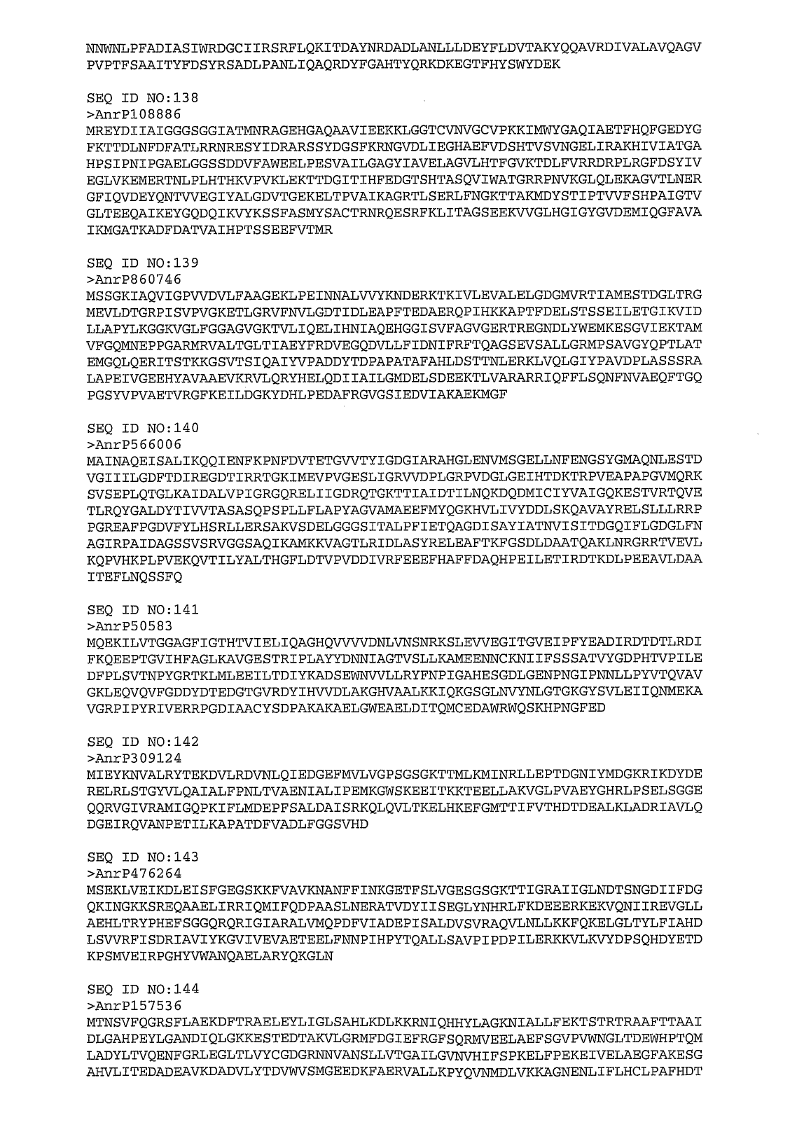

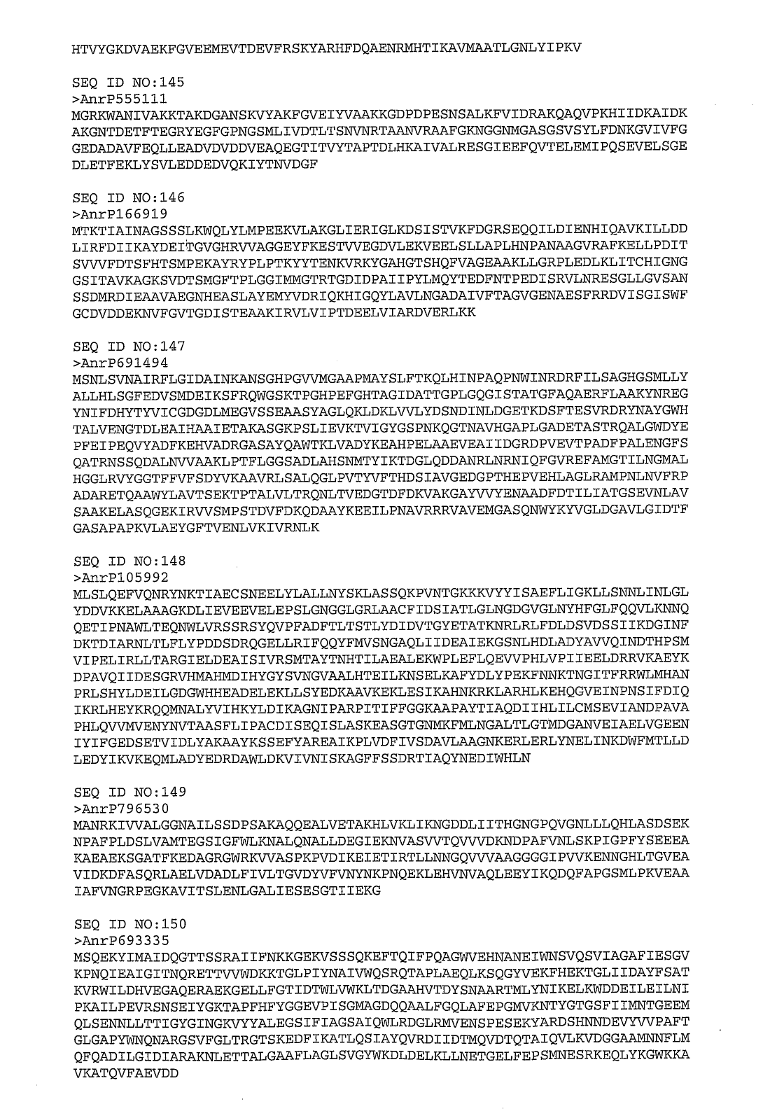

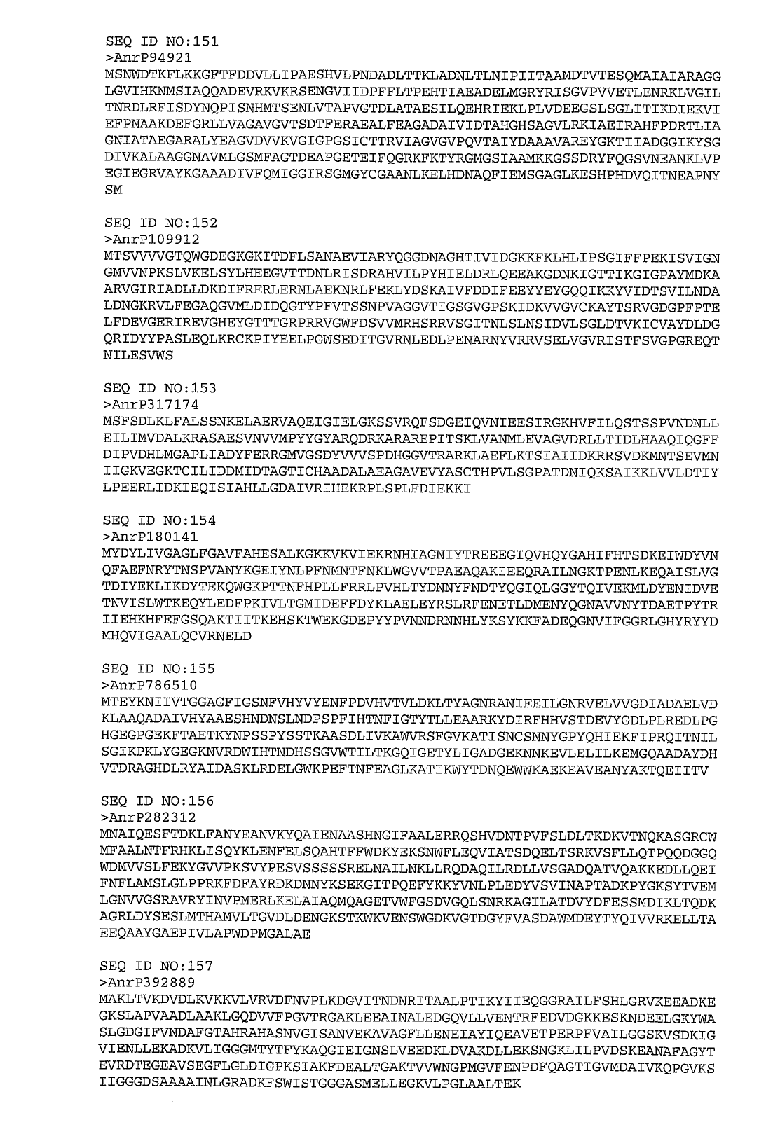

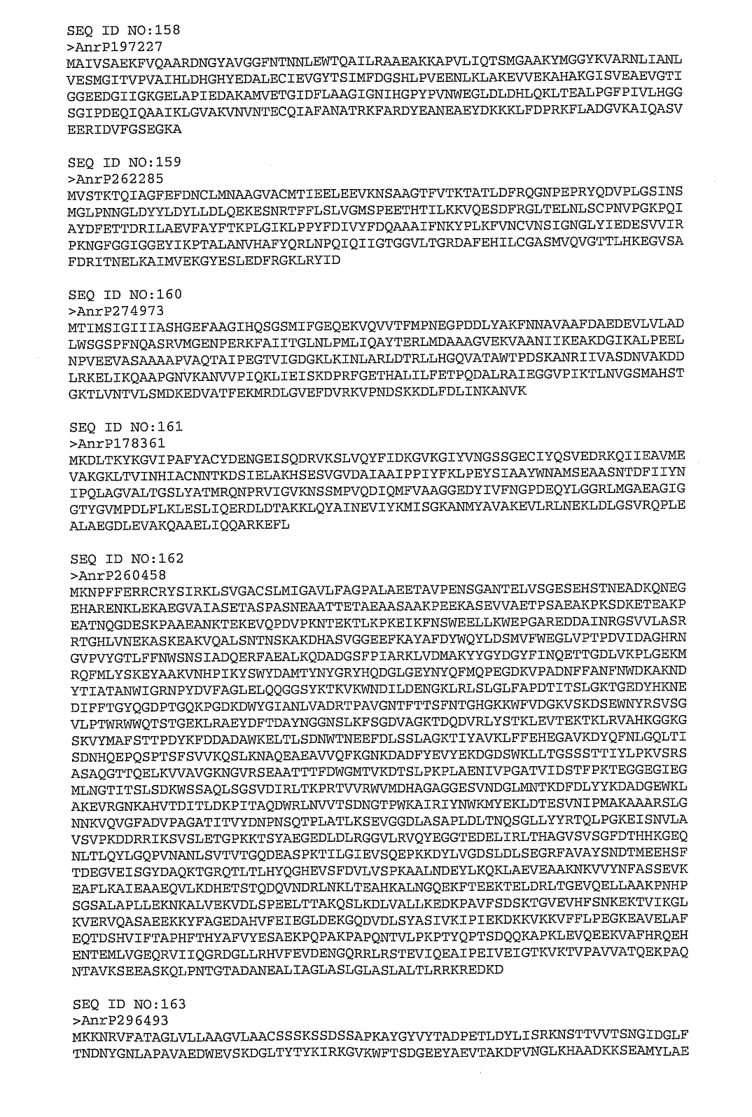

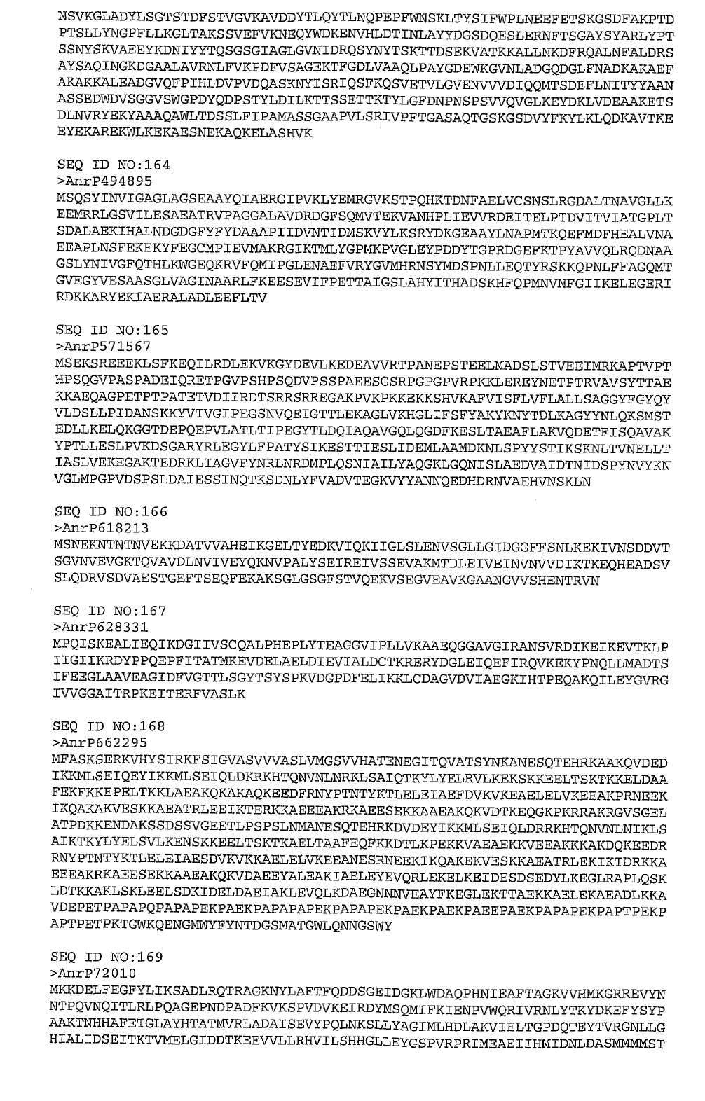

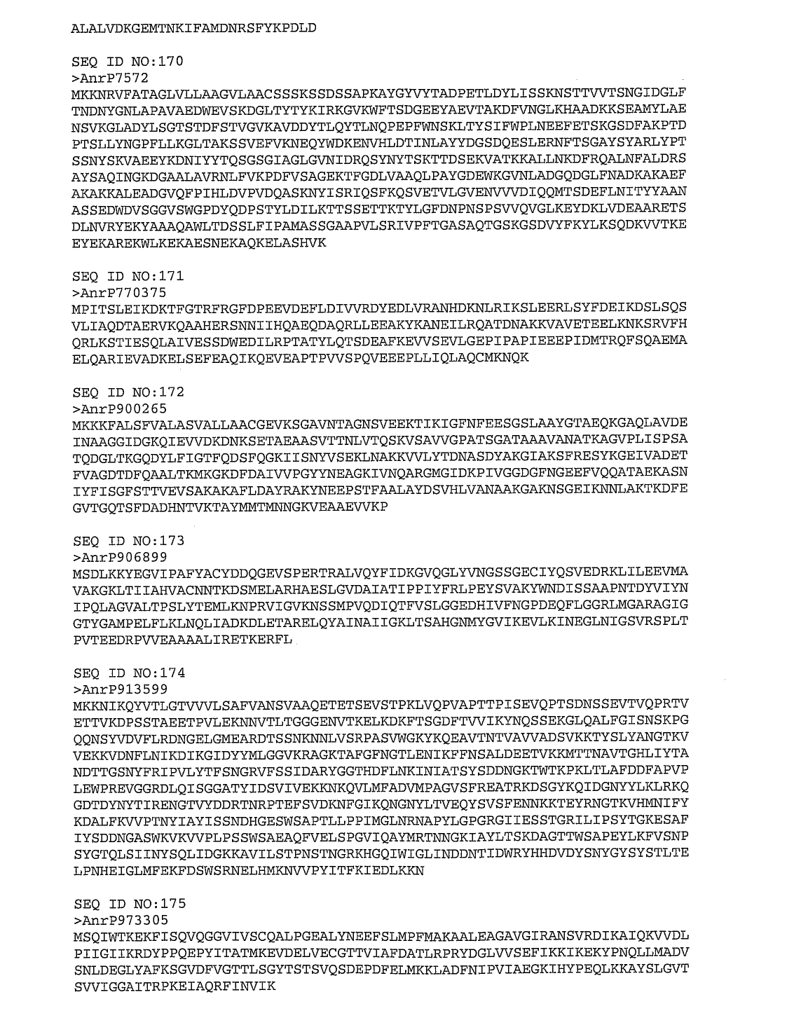

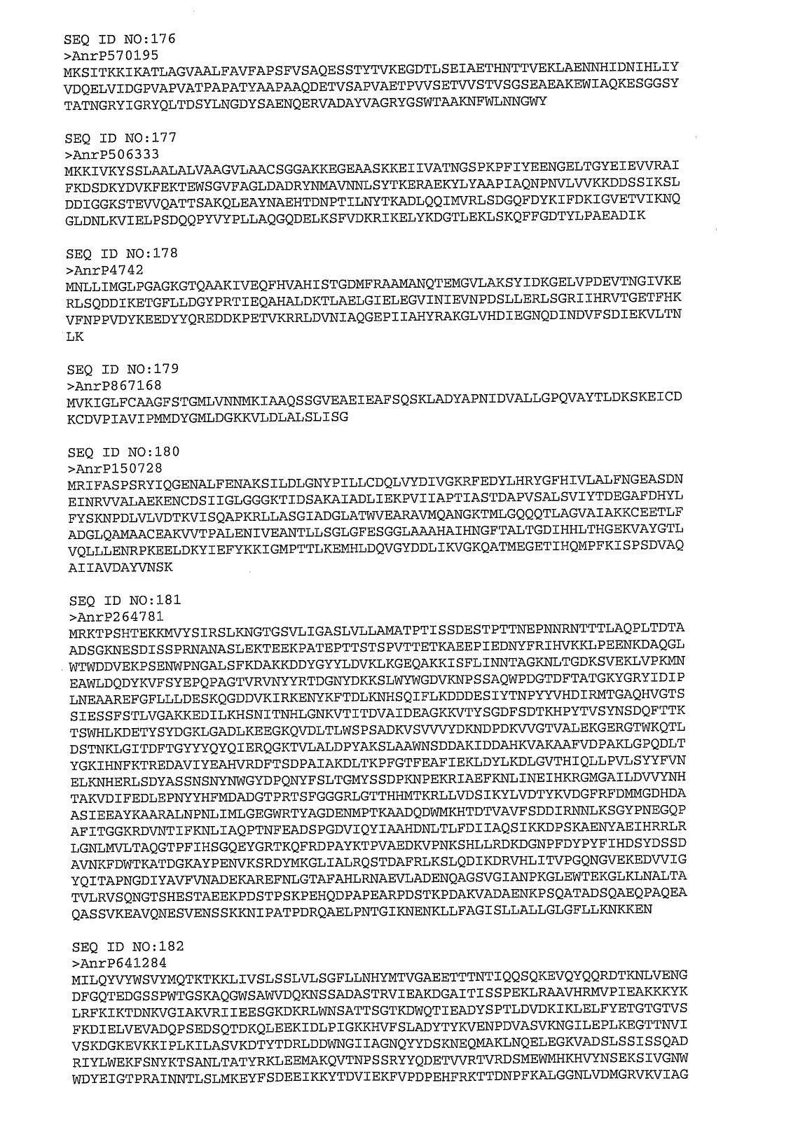

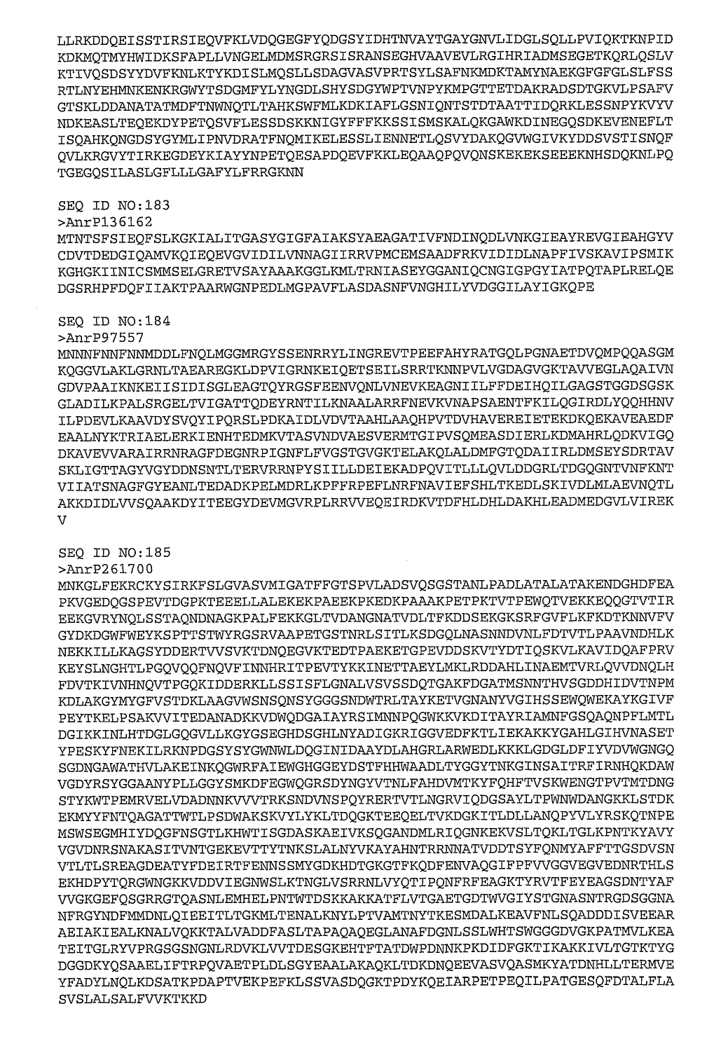

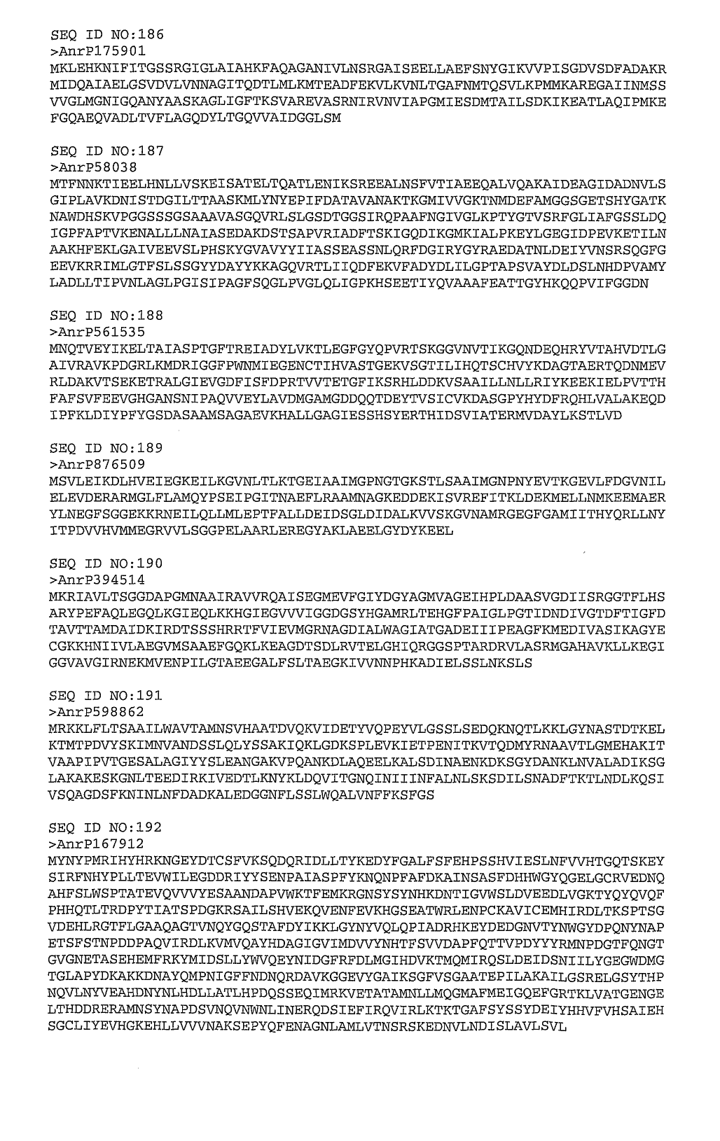

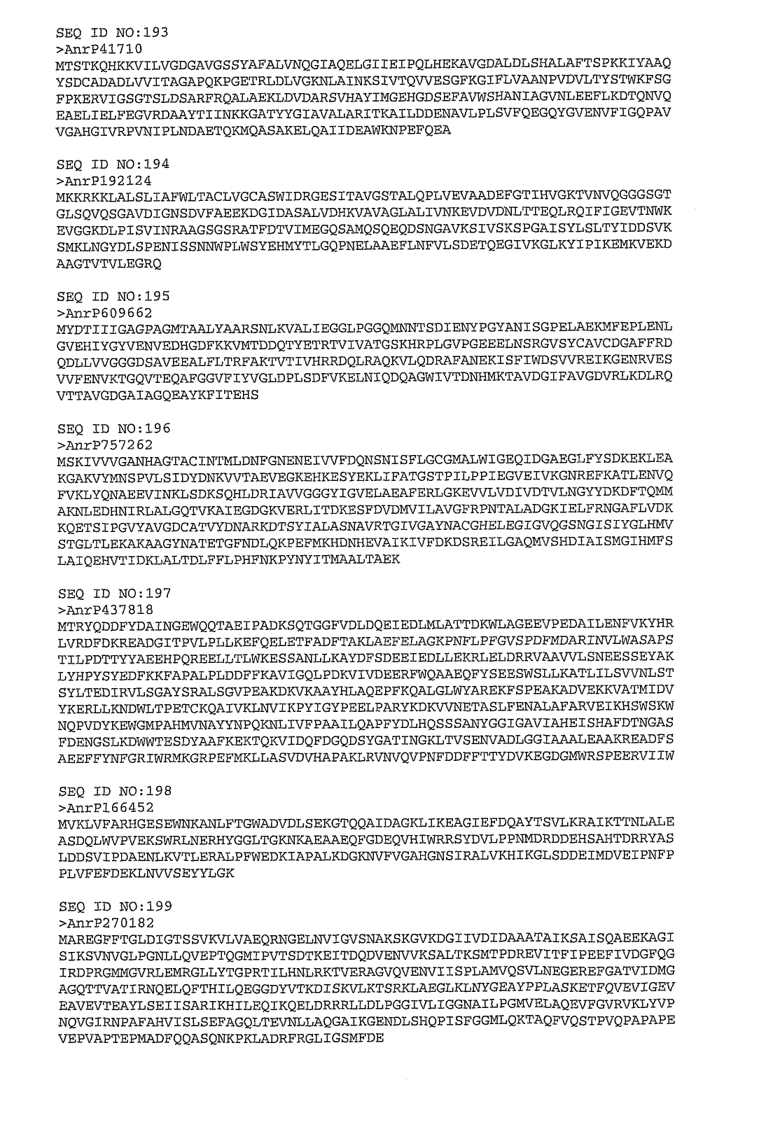

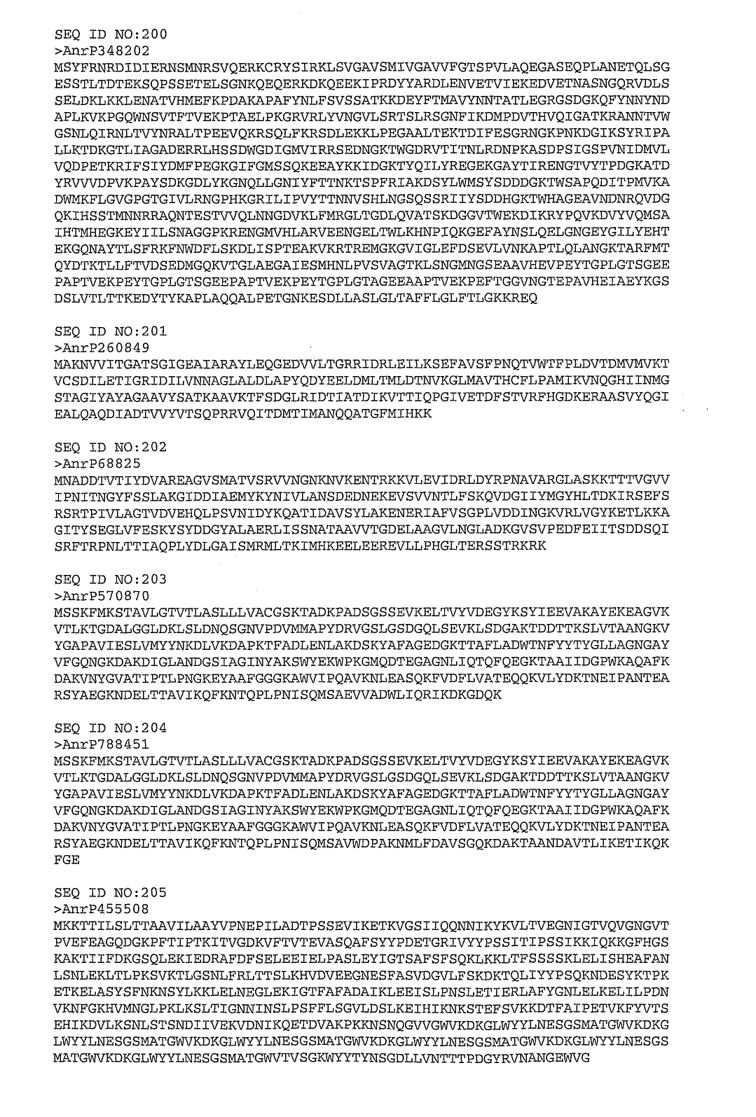

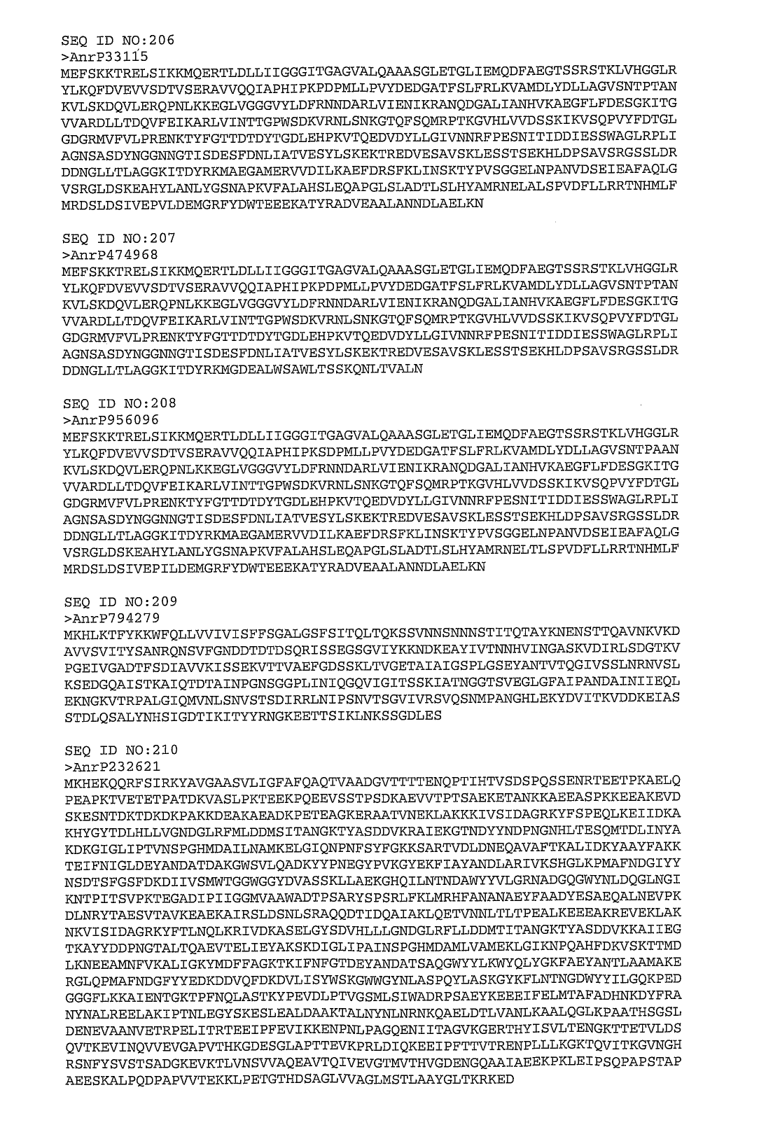

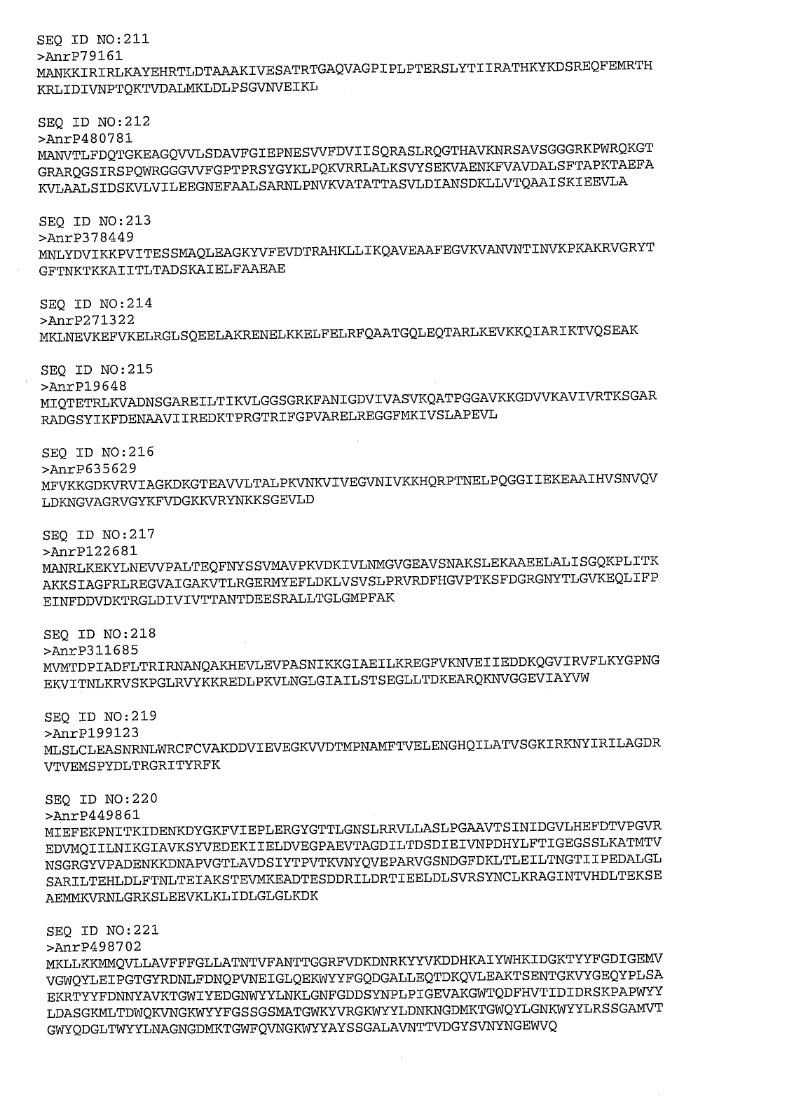

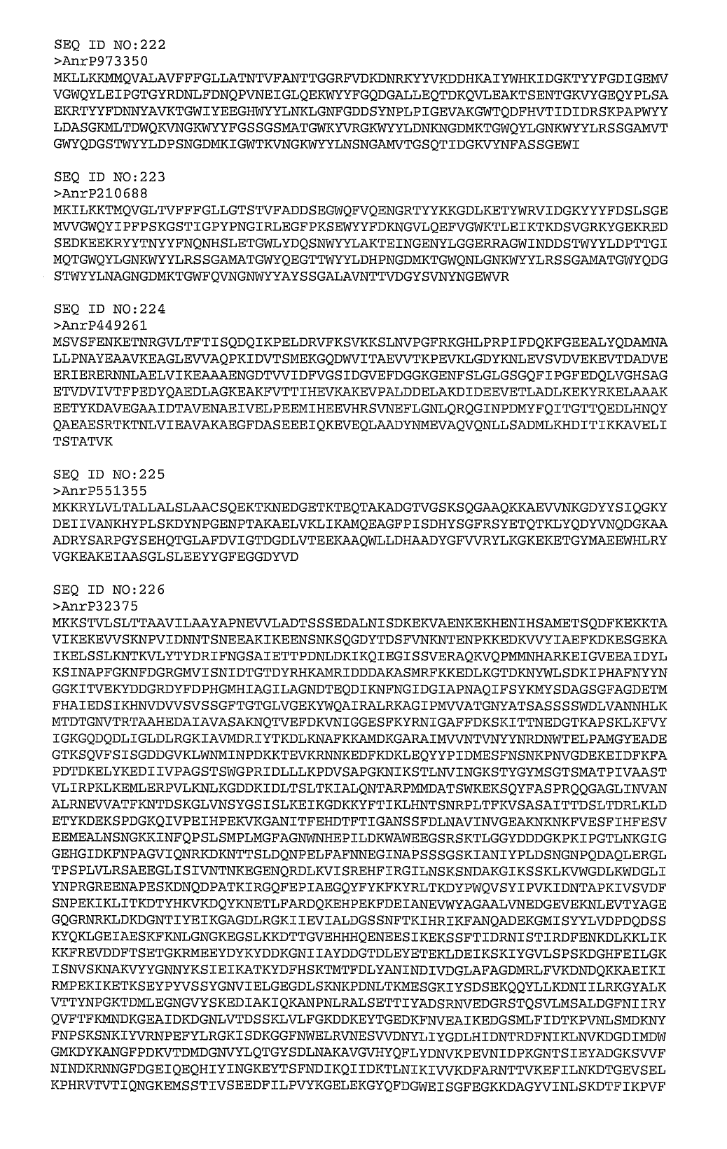

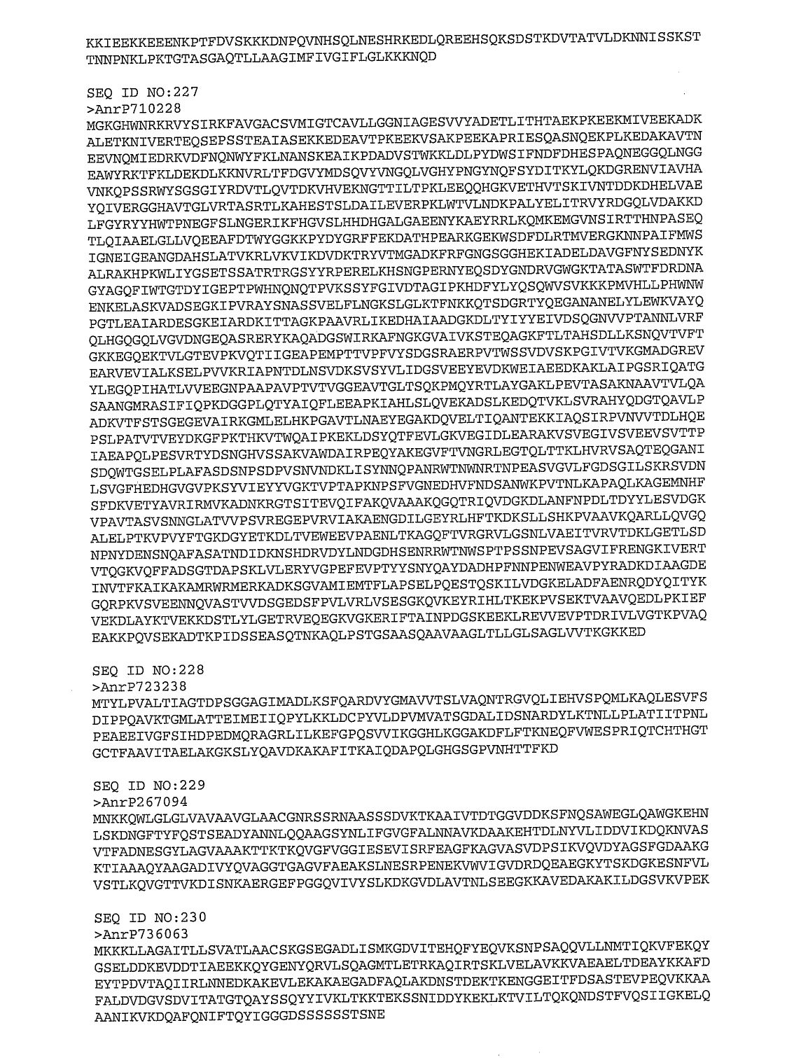

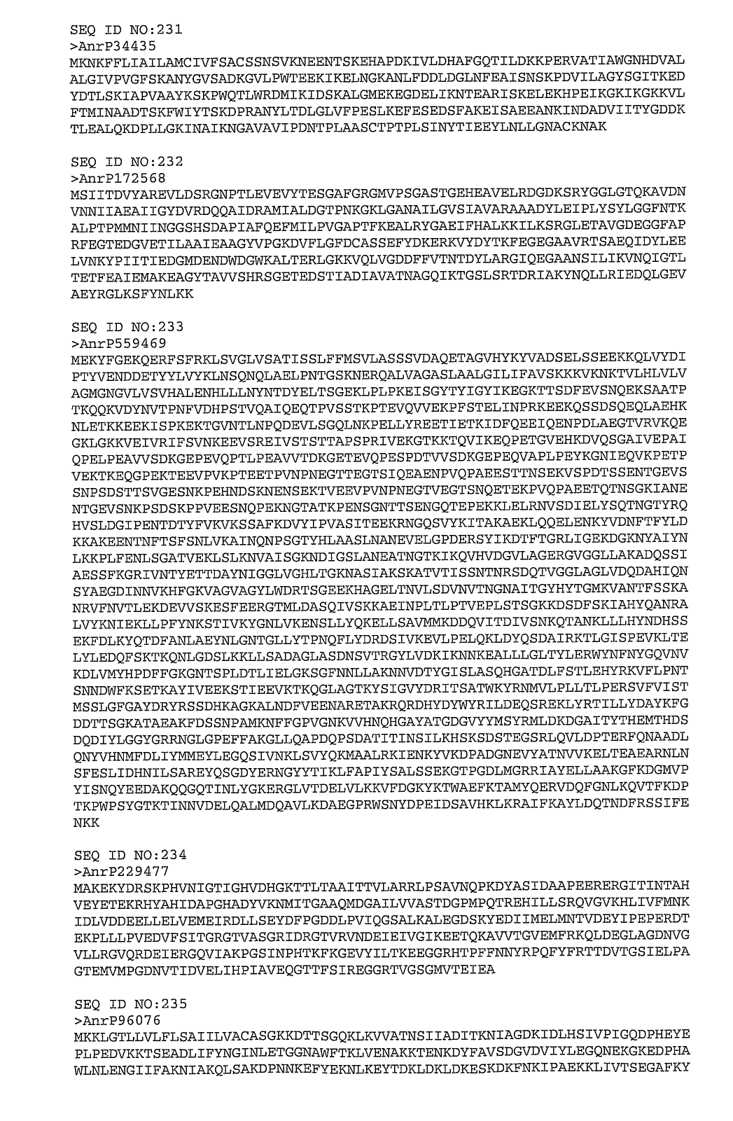

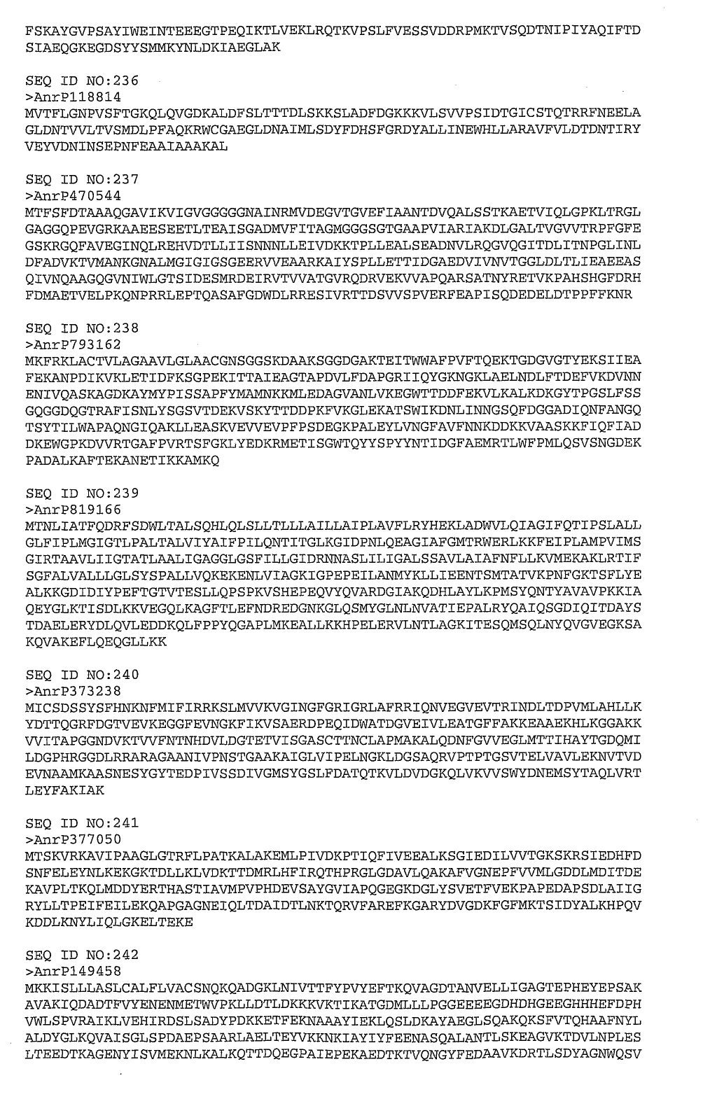

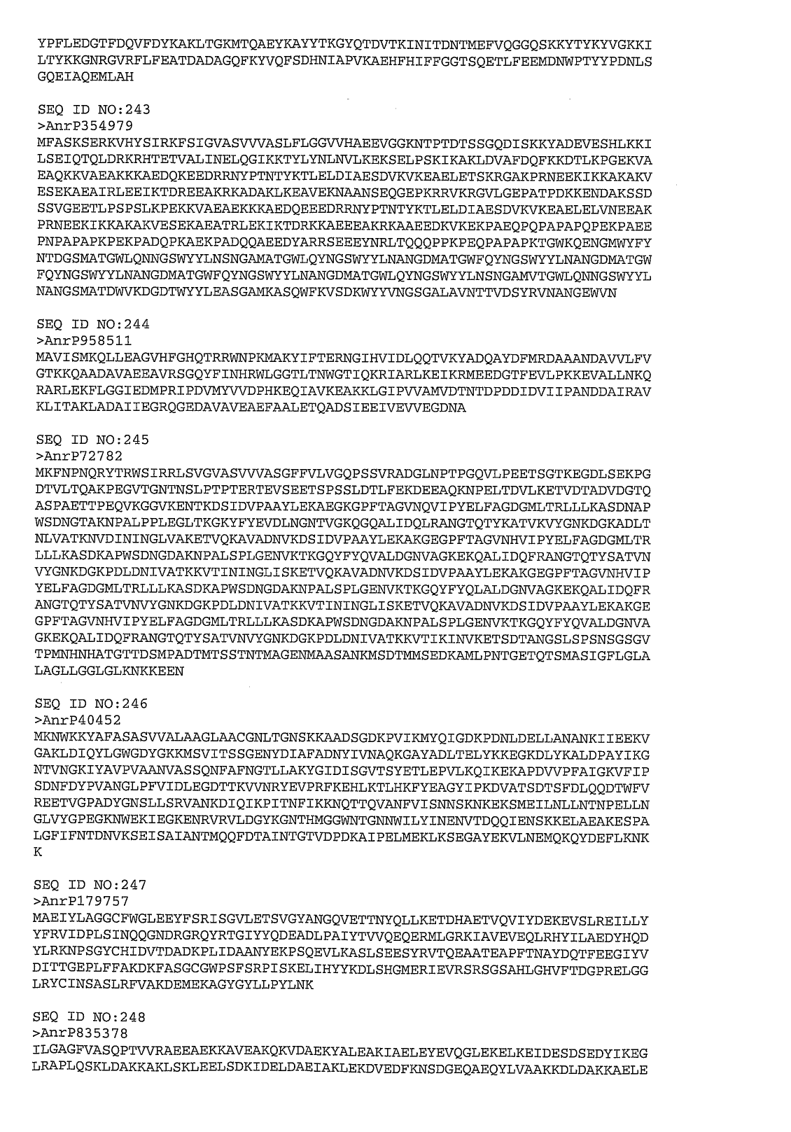

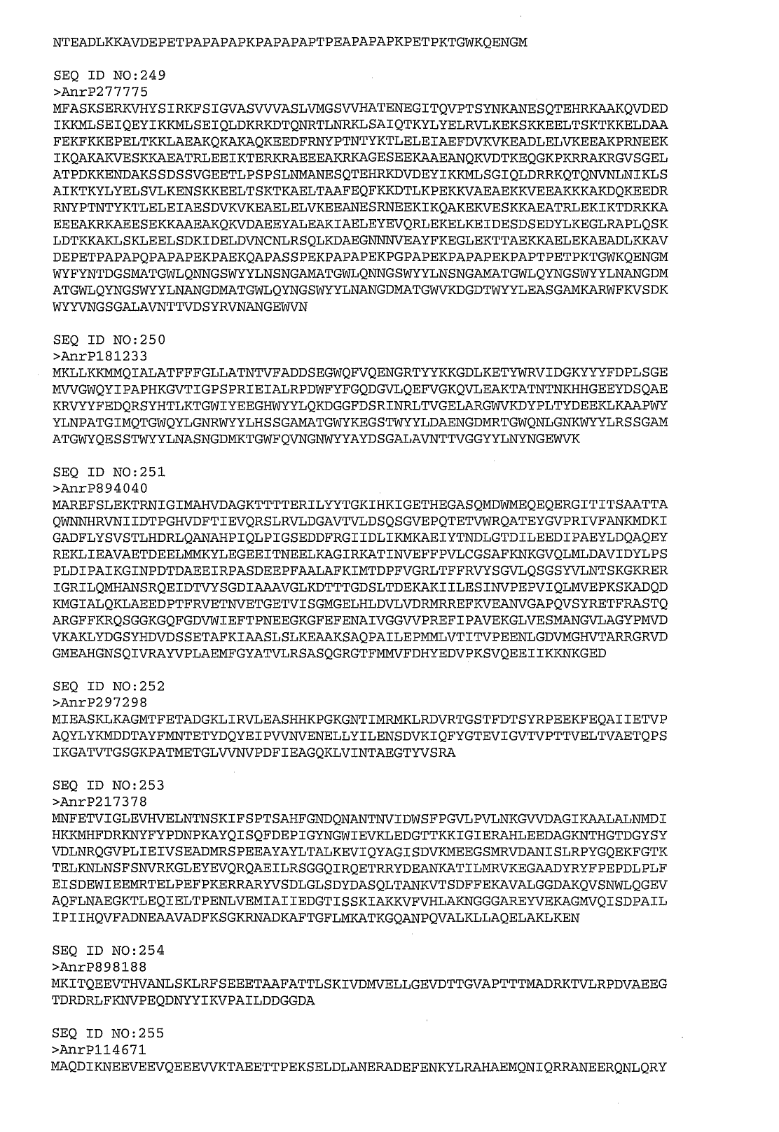

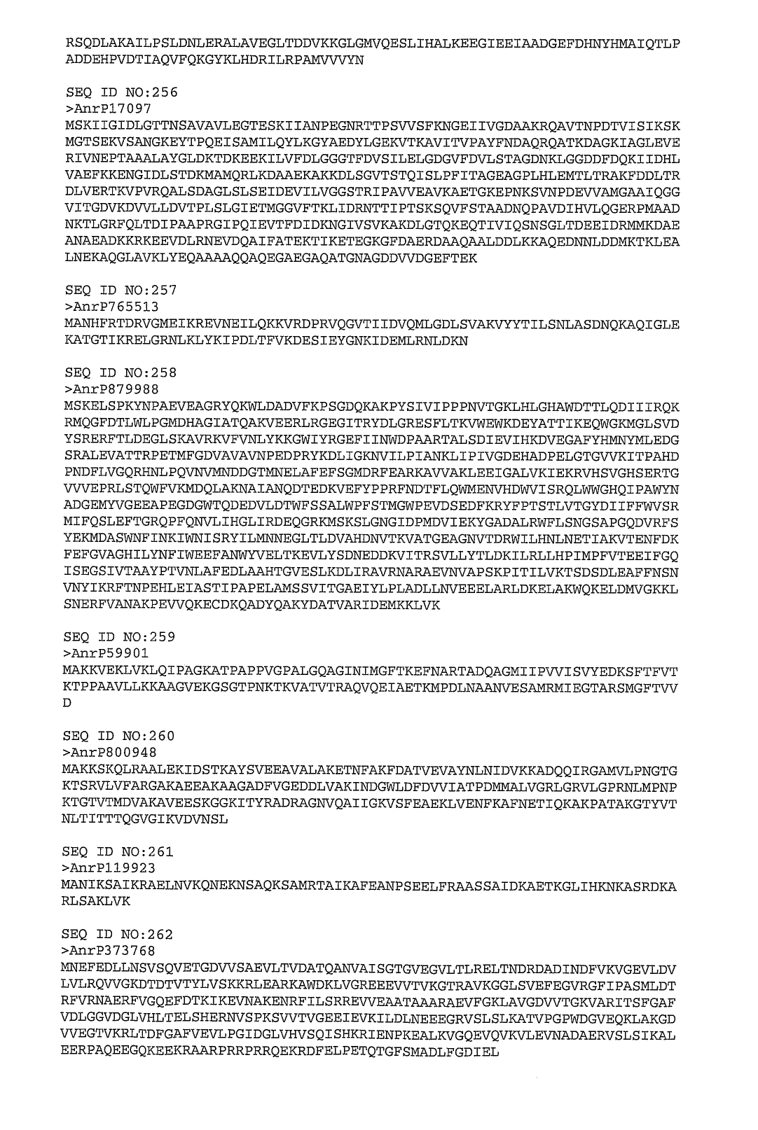

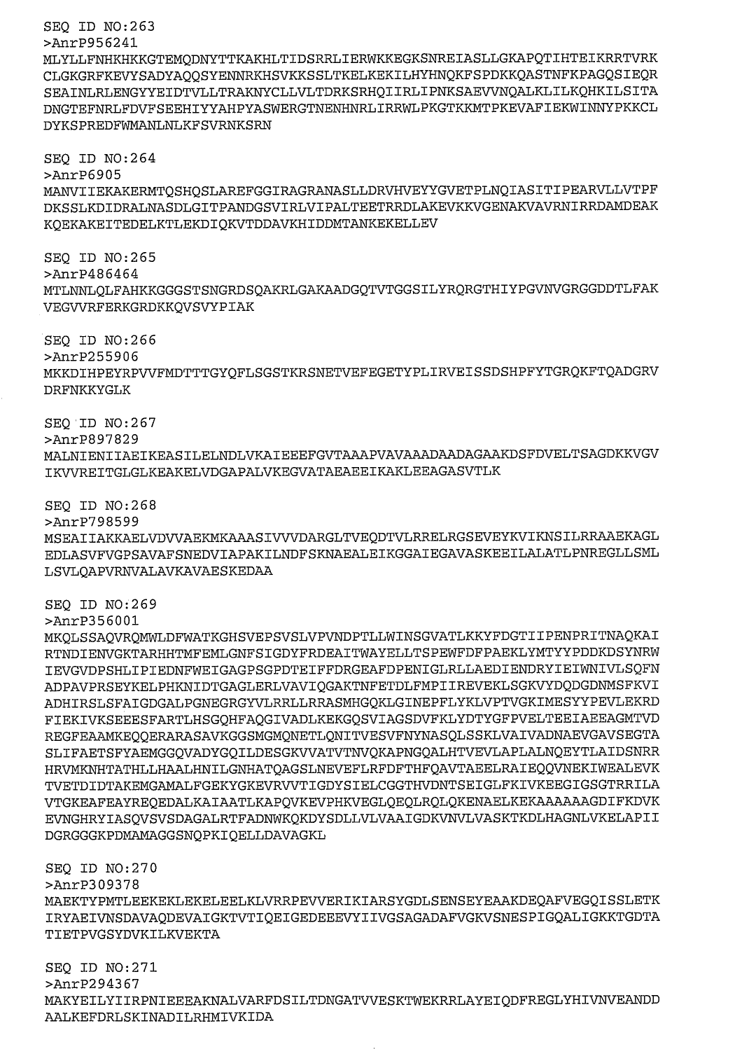

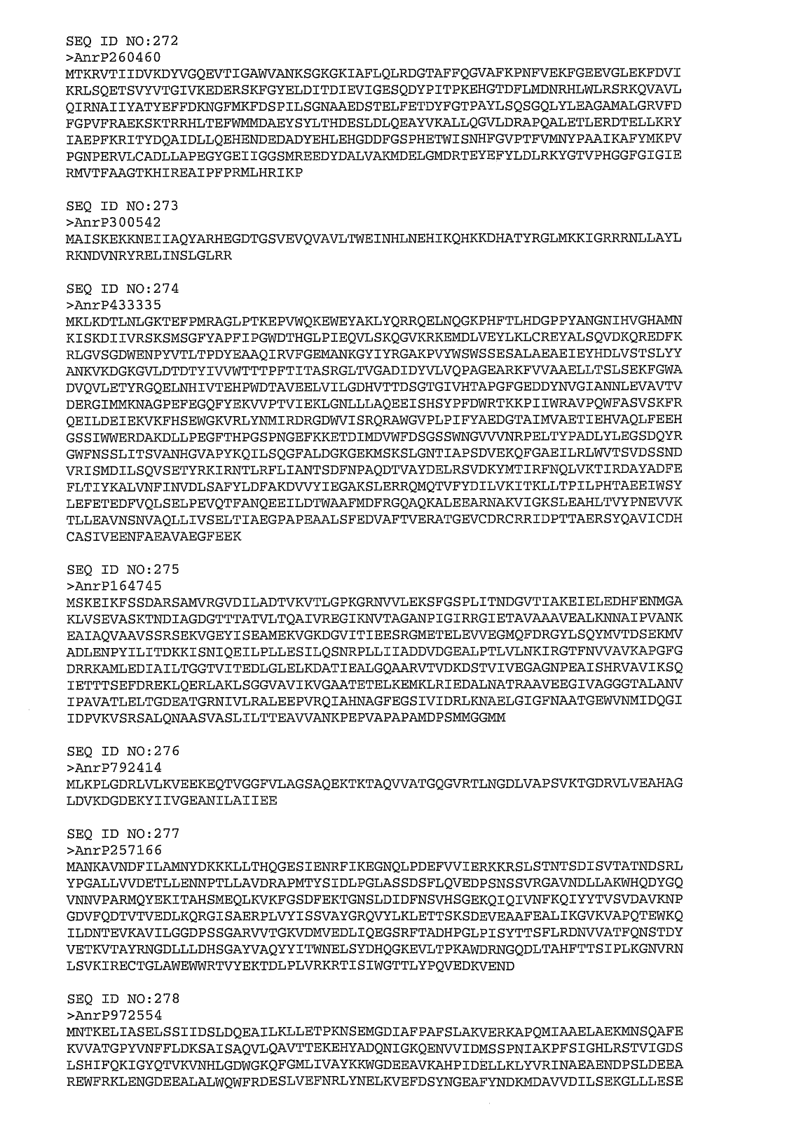

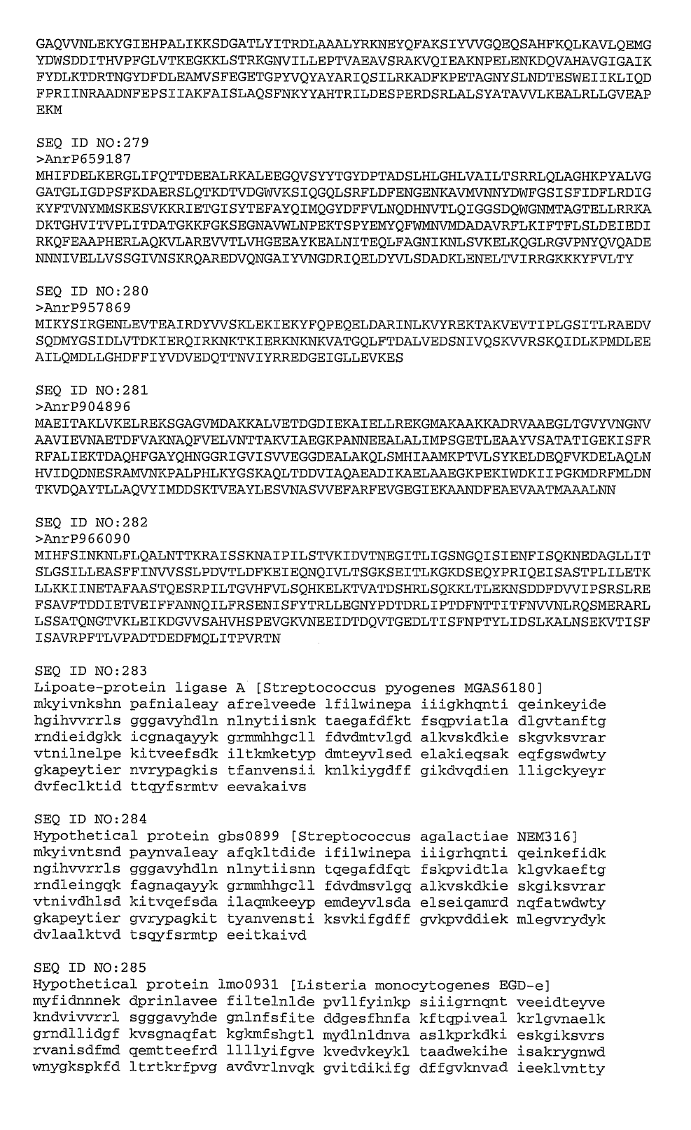

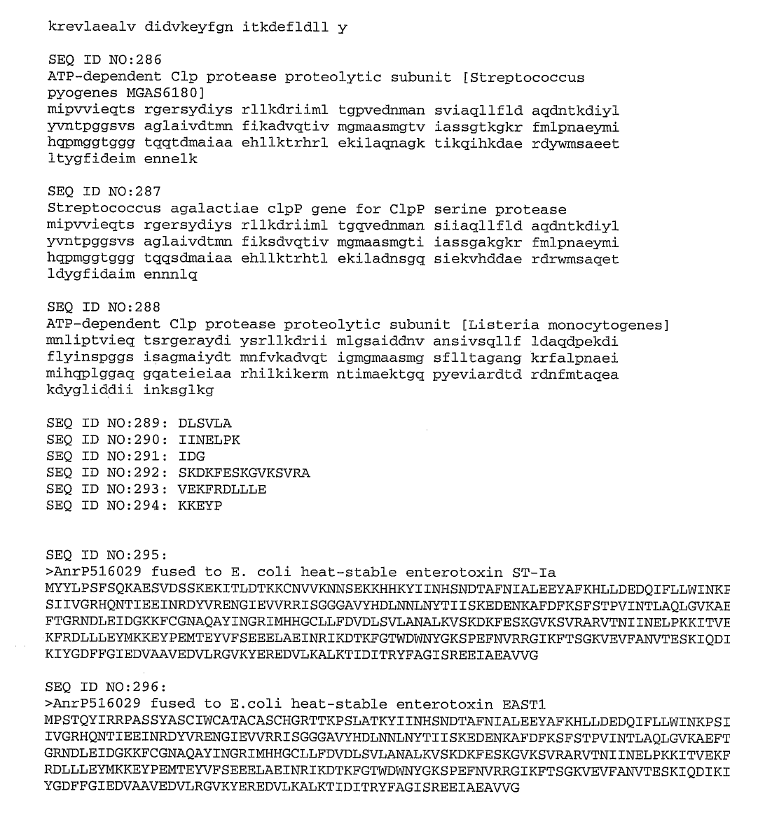

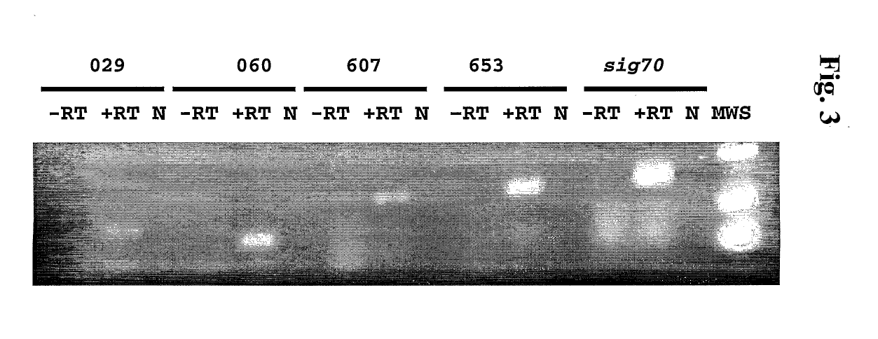

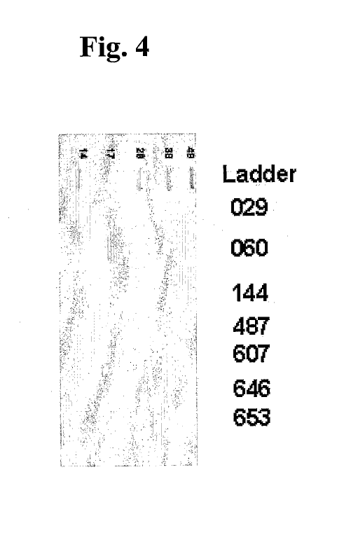

For identification of transcripts of these 4 genes we made a RT-PCR analysis. A BALB/C mouse was infected with S. pneumoniae and total RNA was isolated from spleen after day 1 of infection. RT-PCRs with RNAs from spleen demonstrated that the selected genes are expressed in S. pneumoniae after animal infection (FIG. 3). In addition Western Blots (WB) with patient sera were made for testing the immunogenicity of proteins listed in table 3. Furthermore 3 additional proteins (144, AnrP454144 (14 kDa rec. vac.); 487, AnrP98487 (32 kDa); 646; AnrP373646 (25 kDa)) were also tested for immunogenicity with Western Blot. We detected recombinant vaccines 029, 060, 607, 646 and 653 in immunoblots (WBs) with sera isolated from a single patient (FIG. 4) or pooled from different patients (data not shown). In WBs unspecific signals or no signals were detected for purified proteins 144 and 487, however, lack of a signal does not exclude that these two proteins may be suitable as vaccines.

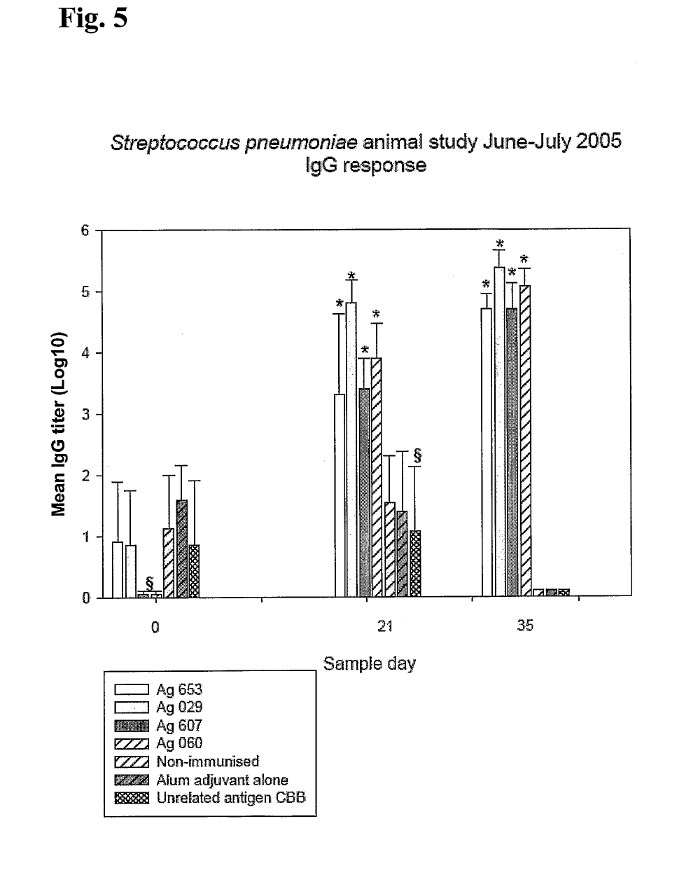

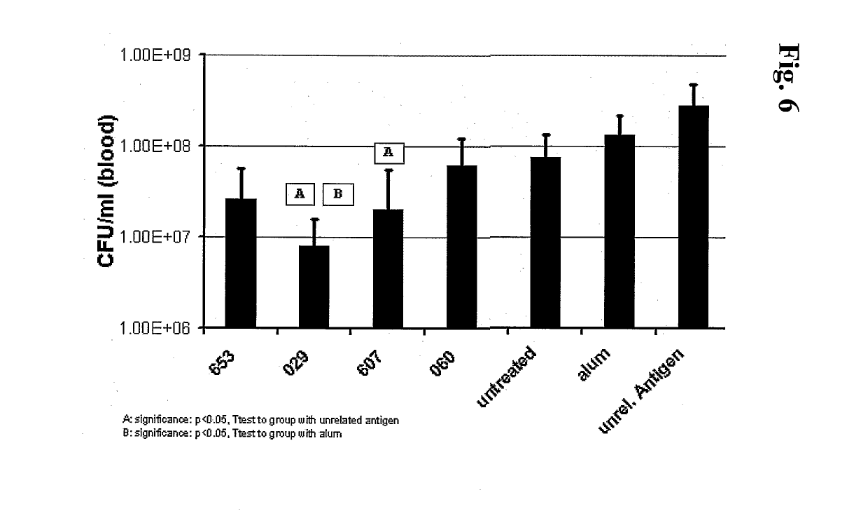

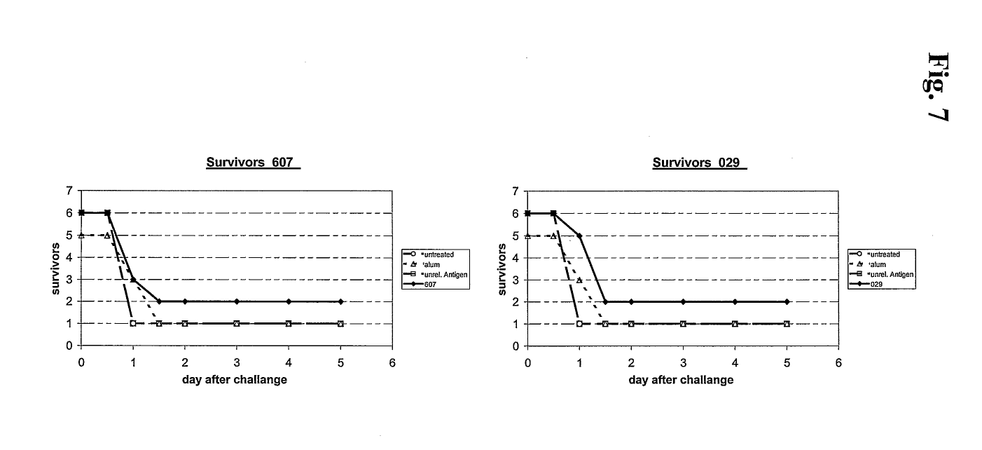

We vaccinated mice with proteins in table 3 as a protein vaccine and tested the protection efficiency against S. pneumoniae. Antigens were prepared with alum and injected subcutaneously into BALB/C mice at three time points (day 0, 14, 28). For negative controls, mice were left untreated (group 5) or treated with 100 kg alum (group 6) or with an unrelated protein (group 7). We tested the immunogenicity of each protein at day 0, day 21 and day 35 of vaccination with an ELISA assay. Mice produced immunoglobulin (IgG) against antigens 029, 060, 607, and 653 (FIG. 5). No immune response against pneumococcal antigens was detected as expected in animals of groups 5, 6 and 7. For the bacterial challenge, each mouse was infected with S. pneumoniae D39 (1 CFU per mouse) at day 35 of vaccination. Mice derived from two groups, vaccinated with proteins 029 and 607 demonstrated a lower mortality and a lower CFU-titre after infection with this S. pneumoniae strain (FIG. 6, FIG. 7). Proteins 060 and 653 also showed a trend towards lower CFU-titre (FIG. 6).

In order to investigate variations in sequence between different strains of Streptococcus pneumoniae, the sequences of antigens 029 and 607 were partially determined from serotypes 15b, 15c, and 35f, and from Streptococcus pneumoniae strain D39. These sequences were compared with database sequences of 029 and 607 from type 4 (TIGR) and R6 (Sanger Center). The region of 029 from amino acid position 1 to amino acid position 315 showed more than 98% sequence identity on amino acid level between the six strains. For 607, more than 98% sequence identity on amino acid level between the six strains was found in the region from position 20 to position 190. These data indicate that 029 and 607 are well-conserved across different strains.

The structure of antigen 029 (SEQ ID NO:16) (putative lipoate protein ligase) has been determined and is accessible under accession number 1VQZ in the PDB (Protein Data Bank) and HSSP databases of EBI, the European Bioinformatics Institute. Surface-exposed regions were predicted by identification of amino acids with high water accessibility (ACC), which at the same time should have a low variability (VAR) in sequence (see table below). The amino acid stretches of different areas separated by several amino acids are adjacent in the 3D structure and are therefore paired together.

### Amino Acids Exposed on the Surface:

| | | | |
|---|---|---|---|
| Amino acid | | | |
| No. | Amino acid | ACC | VAR |

| | | | |
|---|---|---|---|
| 127 | I | 7 | 21 |
| 128 | D | 139 | 26 |
| 129 | G | 36 | 18 |
| 155 | D | 78 | 20 |
| 156 | L | 90 | 24 |
| 157 | S | 72 | 35 |
| 158 | V | 9 | 49 |
| 159 | L | 53 | 14 |
| 160 | A | 83 | 42 |
| 166 | S | 15 | 37 |
| 167 | K | 102 | 35 |
| 168 | D | 59 | 37 |
| 169 | K | 74 | 7 |
| 170 | F | 34 | 22 |
| 171 | E | 92 | 41 |
| 172 | S | 86 | 26 |
| 173 | K | 39 | 13 |
| 174 | G | 64 | 10 |
| 175 | V | 78 | 11 |
| 176 | K | 96 | 28 |
| 177 | S | 77 | 0 |
| 178 | V | 100 | 11 |
| 179 | R | 102 | 20 |
| 180 | A | 86 | 32 |
| 185 | I | 0 | 14 |
| 186 | I | 44 | 48 |
| 187 | N | 110 | 32 |
| 188 | E | 51 | 46 |
| 189 | L | 18 | 18 |
| 190 | P | 102 | 42 |
| 191 | K | 70 | 36 |
| 195 | V | 1 | 43 |
| 196 | E | 84 | 26 |
| 197 | K | 112 | 39 |
| 198 | F | 0 | 22 |
| 199 | R | 37 | 43 |
| 200 | D | 72 | 40 |
| 201 | L | 33 | 42 |
| 202 | L | 0 | 25 |
| 203 | L | 24 | 38 |
| 204 | E | 116 | 46 |
| 207 | K | 65 | 51 |
| 208 | K | 102 | 41 |
| 209 | E | 81 | 50 |
| 210 | Y | 42 | 50 |
| 211 | P | 107 | 46 |

155-160 + 185-191 [DLSVLA (SEQ ID NO:289)/IINELPK (SEQ ID NO:290)]
127-128 + 166-180 [IDG/SKDKFESKGVKSVRA (SEQ ID NO:291)]
195-204 + 207-211 [VEKFRDLLLE (SEQ ID NO:292)/KKEYP (SEQ ID NO:293)]

1. A composition for use as a medicament, comprising one of the group consisting of:
a) an antibody capable of binding a polypeptide selected from the group consisting of
SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282,
b) a polypeptide selected from the group consisting of the polypeptides of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282.
c) an antigenic fragment of said polypeptide,
d) a variant of said polypeptide.
e) a polynucleotide comprising a sequence encoding said polypeptide,
f) an expression vector comprising a sequence encoding said polypeptide, and
g) a recombinant virus or recombinant cell comprising said polynucleotide or said expression vector.

2. The composition of claim 1, wherein a minimum sequence identity of said variant of said polypeptide with said sequence is selected from the group consisting of 95%, 96%, 97% 98%, and 99%.

3. The composition of claim 1, wherein a percentage of the full length of said sequence contained in said antigenic fragment is less than a value selected from the group consisting of 99%, 75%, 50%, 25%, 20%, 15%, and 10%.

4. The composition of claim 1, wherein the number of consecutive amino acid residues of said sequence contained within said antigenic fragment is less than a number selected from the group consisting of 70, 50, 40, 30, and 20.

5. The composition of claim 1, wherein the minimum number of consecutive amino acids of said sequence contained within said antigenic fragment is selected from the group consisting of 6, 7, 8, 9, and 10.

6. The composition of claim 1, wherein the composition comprises at least two polypeptides selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:110, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282.

7-13. (canceled)

14. The composition of claim 1, wherein said polypeptide comprises an affinity tag.

15. The composition of claim 1, wherein said recombinant cell is an attenuated or reduced-virulence Escherichia coli cell or an attenuated or reduced-virulence Salmonella cell.

16. The composition of claim 1, wherein said recombinant cell is alive.

17. The composition of claim 1, wherein said recombinant cell is dead.

18. The composition of claim 1, wherein the medicament is a vaccine.

19. The composition of claim 1, further comprising an immunogenic carrier, wherein the immunogenic carrier is bound to said polypeptide.

20. The composition of claim 1, further comprising an adjuvant.

21. The composition of claim 1, wherein said antibody is capable of binding an intact Streptococcus pneumoniae cell.

22. The composition of claim 1, wherein said antibody is polyclonal.

23. The composition of claim 1, wherein said antibody is monoclonal.

24. The composition of claim 1, wherein said antibody is a human antibody or humanized antibody.

25. The composition of claim 1, wherein the antibody is an antigen-binding fragment of an antibody.

26. The composition of claim 1, wherein the antibody has a dissociation constant Kd of less than a value selected from the group consisting of 5×10⁻⁶ M, 10⁻⁶ M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴Mg, 5×10⁻¹⁵M, 10⁻¹⁵M.

27. The composition of claim 1, wherein said composition additionally comprises a pharmaceutically-acceptable carrier.

28. The composition of claim 1, wherein the composition is suitable for systemic administration.

29. The composition of claim 1, wherein said composition is suitable for a mode of administration selected from the group consisting of intravenous, intramuscular, and subcutaneous administration.

30. The composition of claim 1, wherein said composition is suitable for oral administration.

31. The composition of claim 1, wherein said composition is suitable for intranasal administration.

32. An antibody capable of binding a polypeptide having an amino acid sequence selected from the group consisting of preferably selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282.

33. The antibody of claim 32, wherein said antibody is capable of binding an intact Streptococcus pneumoniae cell.

34. (canceled)

35. A recombinant cell transformed or transfected with a polynucleotide comprising a sequence encoding a polypeptide, said polypeptide comprising one of the group consisting of:
a) a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282:
b) an antigenic fragment of said sequence; and
c) a variant of said sequence.

36. The recombinant cell of claim 35, wherein the recombinant host cell is selected from the group consisting of an Escherichia coli cell and a Salmonella cell.

37. The recombinant cell of claim 35, wherein said recombinant cell is an attenuated or reduced-virulence cell.

38. A method of immunizing an animal against bacterial infections, comprising administering to the animal a composition comprising a component selected from the group consisting of:
a) a polypeptide which comprises a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282,
b) an antigenic fragment of said sequence,
c) a variant of said sequence,
d) a polynucleotide comprising a sequence encoding said polypeptide,
e) an expression vector comprising a sequence encoding said polypeptide, and
f) a recombinant virus or recombinant cell comprising said polynucleotide or said expression vector.

39. The method of claim 38, wherein administration of said composition induces a protective immune response.

40. The method of claim 38, wherein the composition is suitable for parenteral, intravenous, intramuscular, subcutaneous, oral or intranasal administration.

41-45. (canceled)

46. A method for treating or preventing Streptococcus infections in an animal, comprising administering to said animal an antibody capable of binding a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282.

47. (canceled)

48. A method for raising antibodies to a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282 in a non-human animal, comprising the steps of
a. introducing into said animal a composition comprising one of the group consisting of:
i) said polypeptide,
ii) an antigenic fragment of said sequence,
iii) a variant of said sequence,
iv) a polynucleotide comprising a sequence encoding said polypeptide,
v) an expression vector comprising a sequence encoding said polypeptide, and
vi) a recombinant virus or recombinant cell comprising said polynucleotide or said expression vector;
b. raising antibodies in said animal, and
c. isolating the antibodies.

49-50. (canceled)

51. A method for detecting Streptococcus pneumoniae or cellular fragments thereof in a sample, comprising the steps of
a. providing an indicator moiety capable of specifically binding a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69 SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282,
b. contacting said sample with said indicator moiety; and
c. determining whether a signal has been generated by the indicator moiety, thereby detecting whether said sample contains Streptococcus pneumoniae or cellular fragments thereof.

52. The method of claim 51, wherein the indicator moiety is capable of binding an intact Streptococcus pneumoniae cell.

53. The method of claim 51, wherein said indicator moiety does not pass through the membrane of a Streptococcus pneumoniae cell.

54. The method of claim 51 to 53, wherein said indicator moiety comprises an antibody.

55. A method for detecting Streptococcus pneumoniae or cellular fragments thereof in a sample comprising the step of analyzing said sample by mass spectrometry to detect the presence of a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282.

56. A method for identifying a binding partner of a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, and a fragment of any of SEQ ID NOS: 1-282, comprising the steps of
a. providing said polypeptide;
b. contacting said polypeptide with a putative binding partner, and
c. determining whether said putative binding partner is capable of binding to said polypeptide or fragment.

57. A method for identifying a compound with antibacterial activity against Streptococcus pneumoniae, comprising the steps of
a. providing a sensitized cell having a reduced level of a polypeptide selected from the group consisting of SEQ ID NO:1-282, preferably selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282, and
b. determining the sensitivity of said cell to a putative antibacterial compound.

58. A method for identifying an inhibitor of a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:10, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:21, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, and SEQ ID NO:282, comprising the steps of
a. providing two cells that differ in the level of said polypeptide
b. determining the sensitivity of said cells to a putative inhibitor; and
c. ascertaining whether said two cells are differently affected by the presence of said putative inhibitor.

59. The method of claim 58, wherein the putative inhibitor does not pass through the membrane of a Streptococcus pneumoniae cell.

60. The method of claim 58, wherein the determining step comprises a growth assay.

61. The antibody of claim 32, wherein said antibody is polyclonal.

62. The antibody of claim 32, wherein said antibody is monoclonal.

63. The antibody of claim 32, wherein said antibody is a human antibody or humanized antibody.

64. The antibody of claim 32, wherein said antibody is an antigen-binding fragment of an antibody.

65. The antibody of claim 32, wherein the antibody has a dissociation constant Kd of less than a value selected from the group consisting of 5×10⁻⁶M, 10⁻¹⁶M, 5×10⁻¹⁷M, 10⁻¹⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

66. The method of claim 38, wherein the animal is a human.

67. A method for treating or preventing Streptococcus infections in an animal, comprising administering to said animal the antibody of claim 33.

68. A method for treating or preventing Streptococcus infections in an animal, comprising administering to said animal the antibody of claim 61.

69. A method for treating or preventing Streptococcus infections in an animal, comprising administering to said animal the antibody of claim 62.

70. A method for treating or preventing Streptococcus infections in an animal, comprising administering to said animal the antibody of claim 63.

71. A method for treating or preventing Streptococcus infections in an animal, comprising administering to said animal the antibody of claim 64.

72. A method for treating or preventing Streptococcus infections in an animal, comprising administering to said animal the antibody of claim 65.

73. The method of claim 48, additionally comprising purifying said antibodies.

74. The method of claim 48, further comprising selecting antibodies capable of binding an intact Streptococcus pneumoniae cell.

75. The method of claim 48, wherein the animal is a transgenic animal capable of producing human antibodies.

76. The method of claim 57, wherein the determining step comprises a growth assay.
